(19) United States
(12) Patent Application Publication
(10) Pub. No.: US 2024/0425817 A1
(43) Pub. Date: Dec. 26, 2024

(54) ENHANCED DIFFERENTIATION OF BETA CELLS

(71) Applicant: VERTEX PHARMACEUTICALS INCORPORATED, Boston, MA (US)

(72) Inventors: Bryce CAREY, Boston, MA (US); Aleksander SZYMANIAK, Boston, MA (US); Christopher THANOS, Boston, MA (US)

(73) Assignee: VERTEX PHARMACEUTICALS INCORPORATED, Boston, MA (US)

(21) Appl. No.: 18/270,314

(22) PCT Filed: Dec. 28, 2021
(86) PCT No.: PCT/US2021/065413

§ 371 Date: Jun. 29, 2023

Related U.S. Application Data
(60) Provisional application No. 63/131,471, filed on Dec. 29, 2020.

(51) Int. Cl. C12N 5/071; A61K 35/39

(52) U.S. Cl. CPC C12N 5/0676; A61K 35/39; C12N 2500/33; C12N 2500/38; C12N 2501/727

(57) ABSTRACT

Provided herein are, inter alia, compositions and methods for improved production of SC-β cells in vitro. For example, provided are novel formulations and differentiation methods that result in higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, and improved viability, function, and reduced immunogenicity after transplantation. The disclosed compositions and methods can be employed in the manufacture of SC-islets for human therapeutic use.

### CROSS-REFERENCE TO RELATED APPLICATIONS

This application claims the benefit of U.S. Provisional Patent Application No. 63/131,471, filed Dec. 29, 2020, which is hereby incorporated by reference in its entirety.

### BACKGROUND

Transplantation of pancreas or pancreatic islets has been used for treating diabetes, such as type I diabetes. Pancreatic islet transplantation does not need major surgery and the function of the islet grafts can be maintained for years in a recipient. However, a shortage of pancreatic islets donors prevents this therapy from being effectively implemented. Artificial pancreas or pancreatic islets provide an alternative source of transplantable islets.

### SUMMARY

Pancreatic islet transplantation is a promising therapy that can achieve significant clinical benefit for diabetic subjects, for example, subjects with type I diabetes. As there is a limited supply of pancreatic islets sources from donor pancreatic tissue, there is a need for improved techniques to generate implantable islets from alternative sources, such as stem cells. Improved methods of generating islet components (e.g., SC-β cells) could result in more effective therapeutic products (e.g., SC-β cells with improved functionality), improved methods of manufacturing SC-islets for human therapeutic use (e.g., higher cell yields), or a combination thereof.

Provided herein are, inter alia, compositions and methods for improved production of SC-β cells in vitro. For example, provided are novel formulations and differentiation methods that result in higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, and improved viability, function, and reduced immunogenicity after transplantation. The disclosed compositions and methods can be employed in the large scale manufacture of SC-islets for human therapeutic use.

Disclosed herein, in some aspects, is an in vitro composition comprising a plurality of PDX1-positive, NKX6.1-positive, insulin-negative cells, and one or more of an acetyl CoA related metabolite, an HDAC inhibitor, a redox homeostasis regulator, or a one carbon metabolism pathway intermediate.

Disclosed herein, in some aspects, is an in vitro composition comprising a plurality of PDX1-positive, NKX6.1-positive, insulin-negative cells and an acetyl CoA related metabolite.

Disclosed herein, in some aspects, is an in vitro composition comprising a plurality of PDX1-positive, NKX6.1-positive, insulin-negative cells, and one or more of acetate, β-hydroxybutyrate, taurine, or formate.

Disclosed herein, in some aspects, is an in vitro composition comprising a plurality of PDX1-positive, NKX6.1-positive, insulin-negative cells and acetate.

In some cases, the composition comprises between 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate. In some cases, the composition further comprises glutamine. In some cases, the composition comprises from 0.5-20 mM, 0.5-10 mM, 0.5-5 mM, 1-5 mM, 2-5 mM, or 1 mM to 10 mM glutamine.

Disclosed herein, in some aspects, is an in vitro composition comprising a plurality of PDX1-positive, NKX6.1-positive, insulin-negative cells and 3-10, 3-7, 3-8, 3-6, 3-5, 3-4, 3.5-4.5, 3.8-4.2, or 3.9-4.1 mM glutamine.

In some cases, the glutamine is at a concentration of 3.8-4.2 mM. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a dipeptide form. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in an alanine-glutamine dipeptide form. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM of the glutamine is in a free glutamine form. In some cases, the composition further comprises a plurality of insulin-positive endocrine progenitor cells.

Disclosed herein, in some aspects, is an in vitro composition comprising a plurality of insulin-positive endocrine progenitor cells and 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of an acetyl CoA related metabolite.

Disclosed herein, in some aspects, is an in vitro composition comprising a plurality of insulin-positive endocrine progenitor cells and 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate.

In some cases, the composition comprises about 1 mM acetate.

Disclosed herein, in some aspects, is an in vitro composition comprising a plurality of insulin-positive endocrine progenitor cells and 3-10, 3-7, 3-8, 3-6, 3-5, 3-4, 3.5-4.5, 3.8-4.2, or 3.9-4.1 mM glutamine. In some cases, the composition comprises about 4 mM glutamine. In some cases, the composition comprises glutamine and acetate.

Disclosed herein, in some aspects, is an in vitro composition comprising a plurality of insulin-positive endocrine progenitor cells and one or more of an acetyl CoA related metabolite, an HDAC inhibitor, a redox homeostasis regulator, a one carbon metabolism pathway intermediate, glutamate, or L-carnitine.

Disclosed herein, in some aspects, is an in vitro composition comprising a plurality of insulin-positive endocrine progenitor cells and one or more of glutamate, acetate, 3-hydroxybutyrate, taurine, L-carnitine, or formate.

In some cases, the composition comprises 50-1000 nM, 50-800 nM, 50-500 nM, 50-300 nM, 50-250 nM, 100-200 nM, or 125-175 nM of acetate. In some cases, the composition comprises β-hydroxybutyrate. In some cases, the composition comprises taurine. In some cases, the composition comprises formate. In some cases, the composition comprises a vitamin. In some cases, the composition comprises biotin. In some cases, the composition comprises glutamine, acetate, β-hydroxybutyrate, taurine, formate, and biotin. In some cases, the composition further comprises L-carnitine. In some cases, the composition comprises L-carnitine. In some cases, the composition further comprises a BMP signaling pathway inhibitor. In some cases, the BMP signaling pathway inhibitor is LDN193189 or DMH-1 or derivatives thereof. In some cases, the composition further comprises a ROCK inhibitor. In some cases, the ROCK inhibitor is thiazovivin, Y-27632, Fasudil/HA1077, or 14-1152, or derivatives thereof. In some cases, the composition further comprises a histone methyltransferase inhibitor. In some cases, the histone methyltransferase inhibitor is 3-Deazaneplanocin A hydrochloride, Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor, serpin, or derivatives thereof. In some cases, the composition further comprises a TGF-β pathway inhibitor. In some cases, the TGF-β pathway inhibitor is ALK5 inhibitor II, A83-01, 431542, D4476, GW788388, LY364947, LY580276, SB525334, SB505124, SD208, GW6604, or GW788388, or derivatives thereof. In some cases, the composition further comprises a thyroid hormone signaling pathway activator. In some cases, the thyroid hormone signaling pathway activator is GC-1, T3, T1AM, TOAM, Triprop, L-940901, CGS 23425, KB-141, or DITPA, or derivatives thereof. In some cases, wherein the composition further comprises a protein kinase inhibitor. In some cases, the protein kinase inhibitor is staurosporine or Ro-31-8220 or a derivative thereof. In some cases, the composition further comprises a Sonic Hedgehog pathway inhibitor. In some cases, the Sonic Hedgehog pathway inhibitor comprises Sant1, Sant2, Sant 4, Sant4, Cur61414, forskolin, tomatidine, AY9944, triparanol, cyclopamine, or derivatives thereof. In some cases, the composition further comprises a growth factor from epidermal growth factor (EGF) family. In some cases, the growth factor from EGF family comprises betacellulin or EGF. In some cases, the composition further comprises a gamma secretase inhibitor. In some cases, the gamma secretase inhibitor comprises XXI, DAPT, or derivatives thereof. In some cases, the composition further comprises zinc. In some cases, the zinc is in the form of ZnSO4. In some cases, the composition further comprises a serum albumin protein. In some cases, the serum albumin protein is a human serum albumin protein. In some cases, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some cases, the composition further comprises DMEM. In some cases, the DMEM is DMEM/F12. In some cases, the composition does not comprise vitamin C.

In some cases, the plurality of insulin-positive endocrine progenitor cells are dissociated. In some cases, at least 50%, 60%, 65%, 70%, 80%, 85%, 90%, 91%, 92%, 93%, 94%, 95%, 96%, 97%, 98%, 99% or 100% of the plurality of insulin-positive endocrine progenitor cells are not in cell clusters. In some cases, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some cases, the insulin-positive endocrine progenitor cells were previously frozen.

In some cases, at least 30%, 40%, 50%, 60%, 65%, 70%, 80%, 85%, 90%, 91%, 92%, 93%, 94%, 95%, 96%, 97%, 98%, 99% or 100% of the plurality of insulin-positive endocrine progenitor cells are in cell clusters. In some cases, the plurality of insulin-positive endocrine progenitor cells are in cell clusters. In some cases, at least about 40%, 50%, 60%, 70%, 80%, or 90% of the cell clusters have a diameter of from about 50 μm to about 250 μm, from about 75 μm to about 250 μm, or from about 100 μm to about 200 μm. In some cases, at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm. In some cases, the cell clusters have a mean or median diameter of at most 120, at most 130, at most 140, at most 150, at most 160, or at most 170 μm. In some cases, the cell clusters have a mean or median diameter of about 80-150, about 100-150, about 120-150, about 140-150, about 80-130, about 100-130, about 120-130, about 80-120, about 90-120, or about 100-120 μm. In some cases, at least about 50%, at least about 60%, at least about 70%, or at least about 80% of the cell clusters have a diameter of less than 150 μm. In some cases, at least about 50%, at least about 60%, or at least about 70% of the cell clusters have a diameter of less than 130 μm. In some cases, the composition comprises at least 35%, at least 38%, at least 40%, at least 42%, at least 44%, or at least 46% ISL1-positive, NKX6.1-positive cells. In some cases, the composition comprises at most 2%, at most 4%, at most 6%, at most 8%, at most 10%, at most 12%, at most 14%, at most 16%, at most 18%, at most 20%, at most 22%, or at most 22% ISL1-negative, NKX6.1-negative cells.

In some cases, the composition comprises cells that have been engineered to comprise a genomic disruption in at least one gene sequence, wherein said disruption reduces or eliminates expression of a protein encoded by said gene sequence. In some cases, the at least one gene sequence is any one or more of beta-2-microglobulin (B2M), HLA-A, HLA-B, or CIITA. In some cases, the composition comprises cells that have been engineered to have increased expression of PD-L1 and/or CD47. In some cases, the cells have been engineered using a CRISPR/Cas system.

Disclosed herein, in some aspects, is a method comprising contacting a plurality of PDX1-positive, NKX6.1-positive, insulin-negative cells in vitro with a first composition that comprises one or more of an acetyl CoA related metabolite, an HDAC inhibitor, a redox homeostasis regulator or a one carbon metabolism pathway intermediate.

Disclosed herein, in some aspects, is a method comprising contacting a plurality of PDX1-positive, NKX6.1-positive, insulin-negative cells in vitro with a first composition that comprises an acetyl CoA related metabolite.

Disclosed herein, in some aspects, is a method comprising contacting a plurality of PDX1-positive, NKX6.1-positive, insulin-negative cells in vitro with a first composition that comprises one or more of acetate, β-hydroxybutyrate, taurine, or formate.

Disclosed herein, in some aspects, is a method comprising contacting a plurality of PDX1-positive, NKX6.1-positive, insulin-negative cells in vitro with a first composition that comprises acetate.

In some cases, the method comprises contacting the cells with 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate. In some cases, the first composition further comprises glutamine. In some cases, the first composition further comprises 0.5-20 mM, 0.5-10 mM, 0.5-5 mM, 1-5 mM, 2-5 mM, or 1 mM to 10 mM glutamine.

Disclosed herein, in some aspects, is a method comprising contacting a plurality of PDX1-positive, NKX6.1-positive, insulin-negative cells with a first composition that comprises 3-10, 3-7, 3-8, 3-6, 3-5, 3-4, 3.5-4.5, 3.8-4.2, or 3.9-4.1 mM glutamine.

In some cases, the glutamine is at a concentration of 3.8-4.2 mM. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a dipeptide form. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in an alanine-glutamine dipeptide form. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM of the glutamine is in a free glutamine form. In some cases, the first composition comprises about 4 mM glutamine. In some cases, the first composition comprises glutamine and acetate. In some cases, the first composition comprises β-hydroxybutyrate. In some cases, the first composition comprises taurine. In some cases, the first composition comprises formate. In some cases, the first composition comprises a vitamin. In some cases, the first composition further comprises biotin. In some cases, the first composition comprises glutamine, acetate, β-hydroxybutyrate, taurine, formate, and biotin. In some cases, the first composition further comprises a BMP signaling pathway inhibitor. In some cases, the BMP signaling pathway inhibitor is LDN193189 or DMH-1 or a derivative thereof. In some cases, the first composition further comprises a ROCK inhibitor. In some cases, the ROCK inhibitor is thiazovivin, Y-27632, Fasudil/HA1077, or 14-1152, or derivatives thereof. In some cases, the first composition further comprises a histone methyltransferase inhibitor. In some cases, the histone methyltransferase inhibitor is 3-Deazaneplanocin A hydrochloride, Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor, serpin, or derivatives thereof. In some cases, the first composition further comprises zinc. In some cases, the zinc is in the form of ZnSO4. In some cases, the first composition further comprises a serum albumin protein. In some cases, the serum albumin protein is a human serum albumin protein. In some cases, the first composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some cases, the first composition further comprises a TGF-β pathway inhibitor. In some cases, the TGF-β pathway inhibitor is Alk5 inhibitor II, A83-01, 431542, D4476, GW788388, LY364947, LY580276, SB525334, SB505124, SD208, GW6604, or GW788388, or derivatives thereof. In some cases, the first composition further comprises a thyroid hormone signaling pathway activator. In some cases, the thyroid hormone signaling pathway activator is GC-1 or T3, T1AM, TOAM, Triprop, L-940901, CGS 23425, KB-141, or DITPA, or derivatives thereof. In some cases, the first composition further comprises a protein kinase inhibitor. In some cases, the protein kinase inhibitor is staurosporine or Ro-31-8220 or derivatives thereof. In some cases, the first composition further comprises a gamma-secretase inhibitor. In some cases, the gamma-secretase inhibitor is XXI, DAPT, or derivatives thereof.

In some cases, the first composition further comprises L-carnitine. In some cases, the method comprises contacting the plurality of PDX1-positive, NKX6.1-positive cells with the first composition for a period of one to three days, and then with a second composition that comprises one or more of acetate, β-hydroxybutyrate, taurine, or formate for a period of three to seven days.

In some cases, the method comprises contacting the plurality of PDX1-positive, NKX6.1-positive cells with the first composition for a period of one to three days, and then with a second composition that comprises one or more of a TGF-β pathway inhibitor, a thyroid hormone signaling pathway activator, a BMP signaling pathway inhibitor, a ROCK inhibitor, a histone methyltransferase inhibitor, or a protein kinase inhibitor for a period of two to seven days. In some cases, the method comprises contacting the plurality of PDX1-positive, NKX6.1-positive cells with the first composition for a period of one to three days, and then with a second composition that comprises one or more of Alk5i, GC-1, LDN193189, thiazovivin, SSP, or DZNEP for a period of two to seven days. In some cases, the first composition further comprises a Sonic Hedgehog pathway inhibitor. In some cases, the Sonic Hedgehog pathway inhibitor comprises Sant1, Sant2, Sant 4, Sant4, Cur61414, forskolin, tomatidine, AY9944, triparanol, cyclopamine, or derivatives thereof. In some cases, the first composition further comprises a growth factor from epidermal growth factor (EGF) family. In some cases, the growth factor from EGF family comprises betacellulin or EGF. In some cases, the second composition lacks a Sonic Hedgehog pathway inhibitor and a growth factor from epidermal growth factor (EGF) family, and is otherwise same as the first composition.

In some cases, the contacting results in at least a portion of the plurality of PDX1-positive, NKX6.1-positive, insulin-negative cells differentiating into insulin-positive endocrine progenitor cells. In some cases, the insulin-positive endocrine progenitor cells comprise ISL1-positive, NKX6.1-positive cells. In some cases, the contacting results in generation of a cell population that has a higher proportion of ISL1-positive, NKX6.1-positive cells as compared to contacting the plurality of PDX1-positive, NKX6.1-positive, insulin-negative cells with a composition that is otherwise the same as the first composition, but lacks the one or more of acetate, β-hydroxybutyrate, taurine, or formate. In some cases, the contacting results in generation of a population of cells that has at least 35%, at least 38%, at least 40%, at least 42%, at least 44%, or at least 46% ISL1-positive, NKX6.1-positive cells. In some cases, the contacting results in generation of a population of cells that has about 35%, about 38%, about 40%, about 41%, about 42%, or about 47% ISL1-positive, NKX6.1-positive cells. In some cases, the contacting results in a population of cells that has at least 35%, at least 38%, or at least 40% ISL1-positive, NKX6.1-positive cells after 4 days, 7 days, or 10 days in culture. In some cases, the contacting results in generation of a cell population that has a lower proportion of ISL1-negative, NKX6.1-negative cells as compared to contacting the plurality of PDX1-positive, NKX6.1-positive, insulin-negative cells with a composition that is otherwise same as the first composition, but lacks the one or more of acetate, β-hydroxybutyrate, taurine, or formate. In some cases, the contacting results in generation of a cell population that has at most 22%, at most 20%, or at most 18%, at most 16%, at most 14%, at most 12%, at most 10%, at most 8%, at most 6%, or at most 4% ISL1-negative, NKX6.1-negative cells. In some cases, the contacting results in generation of a cell population that has about 2%, 11%, 22%, 20%, or 18% ISL1-negative, NKX6.1-negative cells.

Disclosed herein, in some aspects, is a method comprising contacting a plurality of insulin-positive endocrine progenitor cells in vitro with a first composition that comprises one or more of an acetyl CoA related metabolite, an HDAC inhibitor, a redox homeostasis regulator, a vitamin, a one carbon metabolism pathway intermediate, L-carnitine, or glutamate.

Disclosed herein, in some aspects, is a method comprising contacting a plurality of insulin-positive endocrine progenitor cells in vitro with a first composition that comprises one or more of glutamate, acetate, β-hydroxybutyrate, taurine, biotin, L-carnitine, or formate.

In some cases, the first composition comprises glutamate. In some cases, the first composition comprises acetate. In some cases, the first composition comprises from 50-1000 nM, 50-800 nM, 50-500 nM, 50-300 nM, 50-250 nM, 100-200 nM, or 125-175 nM of acetate.

In some cases, the first composition comprises β-hydroxybutyrate. In some cases, the first composition comprises taurine. In some cases, the first composition comprises formate. In some cases, the first composition further comprises biotin. In some cases, the first composition comprises L-carnitine. In some cases, the first composition comprises glutamate, acetate, 3-hydroxybutyrate, taurine, formate, L-carnitine, and biotin. In some cases, the first composition further comprises a BMP signaling pathway inhibitor. In some cases, the BMP signaling pathway inhibitor is LDN193189 or DMH-1 or derivatives thereof. In some cases, the first composition further comprises a ROCK inhibitor. In some cases, the ROCK inhibitor is thiazovivin, Y-27632, Fasudil/HA1077, or 14-1152, or derivatives thereof. In some cases, the first composition further comprises a histone methyltransferase inhibitor. In some cases, the histone methyltransferase inhibitor is 3-Deazaneplanocin A hydrochloride, Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor, serpin, or derivatives thereof. In some cases, the first composition further comprises a TGF-β pathway inhibitor. In some cases, the TGF-β pathway inhibitor is ALK5 inhibitor II, A83-01, 431542, D4476, GW788388, LY364947, LY580276, SB525334, SB505124, SD208, GW6604, or GW788388, or derivatives thereof. In some cases, the first composition further comprises a thyroid hormone signaling pathway activator. In some cases, the thyroid hormone signaling pathway activator is GC-1, T3, T1AM, TOAM, Triprop, L-940901, CGS 23425, KB-141, or DITPA, or derivatives thereof. In some cases, the first composition further comprises a protein kinase inhibitor. In some cases, the protein kinase inhibitor is staurosporine or Ro-31-8220 or derivatives thereof. In some cases, the first composition further comprises vitamin C. In some cases, the first composition does not comprise vitamin C. In some cases, the first composition further comprises zinc. In some cases, the zinc is in the form of ZnSO4. In some cases, the first composition further comprises a serum albumin protein. In some cases, the serum albumin protein is a human serum albumin protein. In some cases, the first composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some cases, the composition comprises DMEM. In some cases, the DMEM is DMEM/F12.

In some cases, the plurality of insulin-positive endocrine progenitor cells are dissociated. In some cases, the dissociated insulin-positive endocrine progenitor cells were previously frozen. In some cases, the method results in the reaggregation of the dissociated cells into a plurality of cell clusters. In some cases, at least about 40%, 50%, 60%, 70%, 80%, or 90% of the plurality of cell clusters have a diameter from about 50 μm to about 250 μm, from about 75 μm to about 250 μm, or from about 100 μm to about 200 μm. In some cases, at least about 40%, 50%, 60%, 65%, 70%, 75%, 80%, 85%, 95%, or 99% of the cells of the plurality of cell clusters are viable. In some cases, the method results in the reaggregation of the dissociated cells into at least 2, 3, 4, 5, 10, 50, 100, 1000, 10000, 100000, or 1000000 cell clusters. In some cases, at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm. In some cases, the cell clusters have a mean or median diameter of at most 120, at most 130, at most 140, at most 150, at most 160, or at most 170 μm. In some cases, the cell clusters have a mean or median diameter of about 80-150, about 100-150, about 120-150, about 140-150, about 80-130, about 100-130, about 120-130, about 80-120, about 90-120, or about 100-120 μm. In some cases, at least about 50%, at least about 60%, at least about 70%, or at least about 80% of the cell clusters have a diameter of less than 150 microns. In some cases, at least about 50%, at least about 60%, or at least about 70% of the cell clusters have a diameter of less than 130 μm. In some cases, the method is performed over the course of 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days. In some cases, the method is performed over the course of 1-8 days, 1-7 days, 1-6 days, 1-5 days, 1-4 days, 1-3 days, 1-2 days, 2-8 days, 2-7 days, 2-6 days, 2-5 days, 2-4 days, 2-3 days, 3-8 days, 3-7 days, 3-6 days, 3-5 days, 3-4 days, 4-8 days, 4-7 days, 4-6 days, or 4-5 days.

In some cases, the plurality of insulin-positive endocrine progenitor cells is contacted the first composition over the course of about 3 days, 4 days, 5 days, 6 days, 7 days, or 8 days, thereby producing a second population of cells. In some cases, the method further comprises contacting the second population of cells with a second composition that is different from the first composition, thereby differentiating at least a portion of said second population of insulin-positive cells into a third population of cells comprising a plurality of β cells. In some cases, the second population of cells is contacted with the second composition over a course of 1-8 days, 1-7 days, 1-6 days, 1-5 days, 1-4 days, 1-3 days, 1-2 days, 2-8 days, 2-7 days, 2-6 days, 2-5 days, 2-4 days, 2-3 days, 3-8 days, 3-7 days, 3-6 days, 3-5 days, 3-4 days, 4-8 days, 4-7 days, 4-6 days, or 4-5 days. In some cases, the second population of cells is contacted with the second composition over a course of about 3 days, 4 days, 5 days, 6 days, 7 days, or 8 days. In some cases, the second composition does not comprise a BMP signaling pathway inhibitor, a ROCK inhibitor, a histone methyltransferase inhibitor, a TGF-β pathway inhibitor, a thyroid hormone signaling pathway inhibitor, or a protein kinase inhibitor. In some cases, the second composition does not comprise glutamate, acetate, β-hydroxybutyrate, taurine, biotin, L-carnitine, or formate.

Disclosed herein, in some aspects, is a method comprising: (a) contacting a plurality of PDX1-positive, NKX6.1-positive, insulin-negative cells with a first composition in vitro, wherein the first composition comprises acetate, glutamine, and one or more of p-hydroxybutyrate, taurine, formate, or biotin, thereby generating a first population of cells that comprises a plurality of cell clusters that comprise insulin-positive endocrine progenitor cells; (b) dissociating at least a portion of the plurality of cell clusters in the first population of cells in vitro; and (c) contacting the first population of cells comprising at least a portion of the dissociated cell clusters with a second composition in vitro, wherein the second composition comprises one or more of glutamate, acetate, β-hydroxybutyrate, taurine, biotin, L-carnitine, or formate, thereby generating a second population of cells comprising a plurality of cells clusters comprising a plurality of insulin-positive cells. In some cases, the method further comprises: contacting the second population of insulin-positive cells in vitro with a third composition, wherein the third composition is different from the second composition, thereby differentiating at least a portion of said second population of insulin-positive cells into a third population of cells comprising a plurality of 3 cells.

In some cases, the third composition does not comprise glutamate, acetate, 3-hydroxybutyrate, taurine, biotin, L-carnitine, or formate. In some cases, the first composition comprises 3-10, 3-7, 3-8, 3-6, 3-5, 3-4, 3.5-4.5, 3.8-4.2, or 3.9-4.1 mM glutamine. In some cases, the first composition comprises about 4 mM glutamine. In some cases, the first composition comprises 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate. In some cases, the first composition comprises about 1 mM acetate. In some cases, the second composition comprises from 50-1000 nM, 50-800 nM, 50-500 nM, 50-300 nM, 50-250 nM, 100-200 nM, or 125-175 nM of acetate. In some cases, the second composition comprises glutamate, acetate, 3-hydroxybutyrate, taurine, formate, L-carnitine, and biotin.

In some cases, the first composition further comprises at least one agent of a TGF-β signaling pathway inhibitor, a growth factor from EGF family, a retinoic acid signaling pathway activator, a Sonic Hedgehog pathway inhibitor, a thyroid hormone signaling pathway activator, a protein kinase inhibitor, a Rho-associated coiled-coil containing protein kinase (ROCK) inhibitor, a bone morphogenic protein (BMP) type 1 receptor inhibitor, a γ-secretase inhibitor, or a histone methyltransferase inhibitor.

In some cases, the second composition further comprises at least one agent of a TGF-β signaling pathway inhibitor, a retinoic acid signaling pathway activator, a Sonic Hedgehog pathway inhibitor, a thyroid hormone signaling pathway activator, a protein kinase inhibitor, a ROCK inhibitor, a BMP signaling pathway inhibitor, or a histone methyltransferase inhibitor. In some cases, (A) said Sonic Hedgehog pathway inhibitor comprises SANT1; (B) said retinoic acid signaling pathway activator comprises retinoic acid; (C) said γ-secretase inhibitor comprises XXI; (D) said growth factor from the EGF family comprises betacellulin or EGF; (E) said BMP signaling pathway inhibitor comprises LDN193189 or a derivative thereof, (F) said TGF-β signaling pathway inhibitor comprises ALK5 inhibitor II, or a derivative thereof, (G) said thyroid hormone signaling pathway activator comprises GC-1 or T3, or a derivative thereof; (H) said protein kinase inhibitor comprises staurosporine; (I) said ROCK inhibitor comprises thiazovivin, Y-27632, Fasudil/HA1077, or 14-1152, or derivatives thereof; or (J) said histone methyltransferase inhibitor comprises 3-Deazaneplanocin A hydrochloride, or a derivative thereof.

Disclosed herein, in some aspects, is a method comprising contacting a population of NKX6.1-positive, ISL1-positive, insulin-positive cells with one or more of a serum albumin protein, a TGF-β signaling pathway inhibitor, a TH signaling pathway activator, a protein kinase inhibitor, a ROCK inhibitor, a BMP signaling pathway inhibitor, an epigenetic modifying compound, acetyl CoA-related metabolite, a vitamin, histone deacetylase inhibitor (HDACi), a redox homeostasis regulator, a one carbon metabolism pathway intermediate, glutamate, and/or carnitine for a first period of 1, 2, 3, 4, 5, 6, or 7 days (e.g., 4 days). In some cases, the method further comprises contacting the population of cells following the first period with one or more of a serum albumin protein, an acetyl CoA-related metabolite, a vitamin, histone deacetylase inhibitor (HDACi), a redox homeostasis regulator, a one carbon metabolism pathway intermediate, glutamate, and/or carnitine for a second period of 1, 2, 3, 4, 5, 6, or 7 days (e.g., 3 days) or more in the absence of a TGF-β signaling pathway inhibitor, a TH signaling pathway activator, a protein kinase inhibitor, a ROCK inhibitor, a BMP signaling pathway inhibitor, and/or an epigenetic modifying compound.

Disclosed herein, in some aspects, is a method comprising contacting a population of NKX6.1-positive, ISL1-positive, insulin-positive cells with one or more of HSA, Alk5 inhibitor II, GC-1, staurosporine, thiazovivin, LDN193189, DZNEP, taurine, acetate, betahydroxybutyrate, biotin, carnitine, glutamate, and formate for a first period of 1, 2, 3, 4, 5, 6, or 7 days (e.g., 4 days). In some cases, the method further comprises contacting the population of cells following the first period with one or more of HSA, taurine, acetate, betahydroxybutyrate, biotin, carnitine, glutamate, and formate for a second period of 1, 2, 3, 4, 5, 6, or 7 days (e.g., 3 days) or more in the absence of Alk5 inhibitor II, GC-1, staurosporine, thiazovivin, LDN193189, and DZNEP.

In some cases, the method further comprises contacting the population of cells in the first and or second period with ZnSO4. In some cases, the method further comprises contacting the population of cells in the first and or second period with DMEM. In some cases, the DMEM is DMEM/F12. In some cases, the cells are contacted with the same concentration of the serum albumin (e.g., about 0.05% HSA) in the second period as compared to the first period. In some cases, the cells are contacted with a higher concentration of the HSA (e.g., about 1.0%) in the second period as compared to the first period (e.g., about 0.05%). In some cases, the cells have been engineered to comprise a genomic disruption in at least one gene sequence, wherein said disruption reduces or eliminates expression of a protein encoded by said gene sequence. In some cases, the at least one gene sequence is any one or more of beta-2-microglobulin (B2M), HLA-A, HLA-B, or CIITA. In some cases, the composition comprises cells that have been engineered to have increased expression of PD-L1 and/or CD47. In some cases, the cells have been engineered using a CRISPR/Cas system.

Disclosed herein, in some aspects, is a composition comprising insulin-positive endocrine progenitor cells disclosed herein.

Disclosed herein, in some aspects, is a composition comprising at least a portion of the third population of cells of β cells disclosed herein.

Disclosed herein, in some aspects, is a device comprising the cells disclosed herein. In some cases, the device is configured to produce and release insulin when implanted into a subject. In some cases, the cells are encapsulated. In some cases, the device further comprises a semipermeable membrane, wherein the semipermeable membrane is configured to retain the cells in the device and permit passage of insulin.

Disclosed herein, in some aspects, is a method of treating a subject with a disease characterized by high blood sugar levels over a prolonged period of time (e.g., diabetes), the method comprising administering the cells disclosed herein, or implanting the device disclosed herein. In some cases, the disease is diabetes.

Disclosed herein, in some aspects, is a composition comprising a plurality of insulin-positive cells and a cryopreservative, and one or more of an acetyl CoA related metabolite, an HDAC inhibitor, a redox homeostasis regulator, a one carbon metabolism pathway intermediate, a vitamin, or L-glutamine.

In some cases, the composition is cryopreserved. In some cases, the composition comprises an acetyl CoA related metabolite. In some cases, the acetyl CoA related metabolite is acetate. In some cases, the composition comprises 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate. In some cases, the composition comprises glutamine. In some cases, the composition comprises from 0.5-20 mM, 0.5-10 mM, 0.5-5 mM, 1-5 mM, 2-5 mM, 3.8-4.2 or 1 mM to 10 mM glutamine. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in an alanine-glutamine dipeptide form. In some cases, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM of the glutamine is in a free glutamine form. In some cases, the composition comprises a redox homeostasis regulator. In some cases, the redox homeostasis regulator is taurine. In some cases, the composition comprises a one carbon metabolism pathway intermediate. In some cases, the one carbon metabolism pathway intermediate is formate. In some cases, the composition comprises a vitamin. In some cases, the vitamin is biotin.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A and 1B provide the results of a Seahorse assay conducted on human embryonic stem cells incubated for 48 hours with or without MQ media additives as disclosed herein. MQ refers to media comprising formate, acetate, biotin, β-Hydroxybutyrate, taurine, and glutamine. FIG. 1A illustrates the oxygen consumption rate (OCR) through the stages of the assay. FIG. 1B shows the ATP linked oxygen consumption calculated based on the assay. CTL indicates control cells incubated in the absence of MQ. +MQ or Met* indicates cells incubated in the presence of MQ. hESCs cultured in the presence of MQ exhibited an increased basal metabolic rage (FIG. 1A, e.g., see left side of graph), and increased ATP-linked oxygen consumption (FIG. 1), indicating the MQ can alter intracellular metabolism.

FIG. 1C shows bright field microscopy images of cultures of human embryonic stem cells incubated for 48 hours with (MQ) or without (CTL) MQ media additives as disclosed herein, or incubated with MQ additives excluding glutamine and acetate. Cultures incubated with MQ exhibited increased cell density, suggesting enhanced growth. When glutamine and acetate were removed from the MQ cocktail, cell density was reduced compared to the complete set of additives, suggesting that glutamine and acetate contribute to the observed effects of MQ on the cells.

FIG. 2A provides illustrative scatterplots from samples evaluated at the conclusion of stage 5, for cultures incubated with MQ (+Metabolites) or in the absence of MQ (control). SC-β cells are shown in the upper right quadrant (See Nkx6.1+/Isl1+). The addition of MQ to media throughout stage 5 boosted the percent of SC-β cells substantially, from 25.9% in the control culture to 38.1% in the culture with MQ.

FIG. 2B quantifies the percentage of Nkx6.1+/Is11+SC-β cells obtained at the end of stage 5 from repeats conducted without MQ, showing that the SC-3% achieved in the absence of MQ is reproducibly lower than that observed with MQ in FIG. 2A. These data suggest that the addition of MQ during stage 5 can enhance SC-β cell differentiation.

FIG. 3 provides illustrative flow cytometry results from an experiment with MQ absent throughout stage 5 (control), present throughout stage 5 (+MQ), or with MQ additives except acetate and glutamine added (+MQ* -acetate -Q). The percent of Nkx6.1+/Isl1+SC-β cells was 29.8% in the control condition, 41.6% in the presence of MQ, and 34.3% with MQ additives except acetate and glutamine.

FIG. 4 provides summary data from two experiments comparing the percent of SC-β cells present at the end of stage 5 following incubation in the presence or absence of MQ throughout stage 5.

FIG. 5 provides illustrative flow cytometry data from stage 6 day 4, showing that 47.3% of cells were Nkx6.1+/Isl1+ for the culture where MQ was present in stage 5 (+Metabolites), compared to 37% for the control culture.

FIG. 6A demonstrates the percent recovery of all cells on days 4 and 10 of stage 6, relative to the number of viable cells seeded at the beginning of stage 6. The yield of total cells was higher for cultures in which MQ was present in stage 5.

FIG. 6B quantifies the number of SC-β cells on day 4 of stage 6, calculated from the total cell yield times the percent of cells that were SC-β cells. The yield of SC-β cells was higher for cultures in which MQ was present in stage 5.

FIG. 7A shows illustrative flow plots of cells analyzed on day 7 of stage 6 that were cultured in the presence or absence of MQ during stage 5. It was found that 45.1% of cells were Nkx6.1+/Is11+ for the culture where MQ was present in stage 5, compared to 34.3% for cells not incubated with MQ.

FIG. 7B shows the percentage of Nkx6.1+/Isl1+ cells on days 4, 7, and 10 of stage 6, for cells that were cultured in the presence or absence of MQ during stage 5, demonstrating that the high percent of SC-β cells observed with stage 5 MQ treatment is maintained over time (MQ St5).

FIG. 8 illustrates the percent recovery (yield) of all cells on days 4, 7, and 10 of stage 6, relative to the number of viable cells seeded at the beginning of stage 6. Cells were cultured in the presence (MQ) or absence (Control) of MQ, and thawed into medium A or control medium 1 in stage 6. Higher yield was observed for cells that were incubated with MQ in stage 5 (MQ St5).

FIG. 9A provides bright field microscopy images of cells that were incubated in the presence or absence (control) of MQ during stage 5, then thawed into medium A or control medium 1 in stage 6. Images were taken on day 4 of stage 6. Cells that had been incubated with MQ during stage 5 then thawed into medium A formed smaller clusters.

FIG. 9B provides bright field microscopy images of cells that were incubated in the presence or absence (control) of MQ during stage 5, then thawed into medium A or control medium 1 in stage 6. Images were taken on day 7 of stage 6. Cells that had been incubated with MQ during stage 5 then thawed into medium A formed smaller clusters.

FIG. 10 quantifies cluster size frequency on day 7 of stage 6, showing cluster size is smaller for the cells incubated with MQ in stage 5, then thawed into medium A in stage 6. Cells that had been incubated with MQ during stage 5 then thawed into medium A formed smaller clusters.

FIG. 11 shows a stage 6 media (“medium A”) and the components compared to the control medium 1 and control medium 2. S5d6 factors comprise Alk5i (10 μM), GC-1 (1 μM), LDN-193189 (100 nM), thiazovinin (2.5 μM), SSP (3 nM), and DZNEP (100 nM) for days 1-4 of stage 6.

FIG. 12A is a bar graph showing the number of viable cells recovered at S6d7 and S6d11 from control medium 1, control medium 2, medium A (without metabolites), or medium A (with metabolites) cultures. The data show the medium A media using DMEM/F12 further improves viable cell count throughout stage 6.

FIG. 12B is a bar graph showing the percent of cells recovered at S6d7 and S6d11 from control medium 1, control medium 2, medium A (without metabolites), or medium A (with metabolites) cultures. The data show the medium A media using DMEM/F12 further improves cell recovery throughout stage 6.

FIG. 13 is a microscopy image of SC-islet clusters at S6d7 and S6d11 from control medium 1, control medium 2, or medium A (with metabolites) cultures. The images show that the medium A clusters exhibit more homogeneity throughout stage 6.

FIG. 14 shows a series of area graphs showing the frequency and cluster size of cell clusters at S6d4, S6d7, and S6d11 from control medium 1, control medium 2, and medium A cultures. The results show that the medium A clusters are smaller and exhibit greater homogeneity at S6d11.

FIG. 15 is a bar graph showing the GSIS function of S6d11 cells from control medium 1 culture.

FIG. 16 is a bar graph showing the GSIS function of S6d11 cells from control medium 2 culture. The results show that SC-islets in control medium 2 do not exhibit GSIS function up to that of control medium 1 (compare to FIG. 15).

FIG. 17 is a bar graph showing the GSIS function of S6d11 cells from medium A (without metabolites) culture. The results show that SC-islets in medium A (without metabolites) media exhibit improved GSIS function (compare to FIG. 15).

FIG. 18 is a bar graph showing the GSIS function of S6d11 cells from medium A (with metabolites) culture. The results show that SC-islets in medium A media exhibit similar GSIS function to control medium 1 cultures (compare to FIG. 15).

FIG. 19 is a table showing the effect of the medium A. Medium A shows improved cell recovery at S6d11 compared to control medium 2, and improved GSIS function compared to the Control medium 1.

FIG. 20A shows the viability of SC-islet cells 6 weeks after transplantation into nude rats, and compares cells generated by regimen 1 at stage 6 of differentiation to cells generated by a regimen 2 at stage 6. Cells generated using regimen 1 in stage 6 exhibited higher viability compared to cells generated using regimen 2, when evaluated 6 weeks post-implant. FIG. 20B shows the viability of SC-islet cells 6 and 13 weeks after transplantation into nude rats at stage 6 of differentiation generated by a regimen 2 at stage 6.

FIG. 21 quantifies the number of multi-nuclear giant cells (MNGs) detected upon histopathological evaluation of devices 6 weeks after transplantation into nude rates. SC-islets generated by a regimen 1 at stage 6 of differentiation are compared to SC-islets generated by regimen 2 at stage 6. Fewer multi nuclear giant cells (MNGs) were observed at 6 weeks post-implant for cells generated using regimen 1.

FIG. 22 shows illustrative histopathology images of H&E stained sections from nude rats 6 weeks after implantation of devices of the disclosure containing SC-islets generated by methods that comprised regimen 2 or regimen 1 at stage 6 of differentiation. Reduced immune infiltrate and fewer multi nuclear giant cells (MNGs) were observed at 6 weeks post-implant for cells generated using regimen 1.

FIG. 23 illustrates the non-fasted blood glucose levels of diabetic NOD Scid Gamma (NSG) mice that received one (1×) or two (2×) devices loaded with SC-islets generated using regimen 2 or regimen 1 during stage 6 of differentiation. Animals that received two devices (2×) maintained euglycemia by ˜8 weeks post-implant.

FIG. 24A illustrates non-fasted human C-peptide levels over time in diabetic NOD Scid Gamma (NSG) mice that received one (1×) or two (2×) devices loaded with SC-islets generated using the regimen 2 or regimen 1 during stage 6 of differentiation. In contrast to naïve control and diabetic control mice, in which C-peptide levels were undetectable, increases in C-peptide were observed in all experimental groups following implant, with sustained levels observed for animals that received two devices. CAD refers to a cell-housing device.

FIG. 24B compares fasted levels of human C-peptide measured on day 153 and non-fasted levels of C-peptide measured on day 112 post-transplant in diabetic NOD Scid Gamma (NSG) mice that received one (1×) or two (2×) devices loaded with SC-islets generated using the regimen 2 or regimen 1 during stage 6 of differentiation. The data show that SC-islets generated by methods generated using compositions and methods of the disclosure, including regimen 2 and regimen 1, can exhibit efficacy in increasing insulin production upon transplantation into diabetic subjects.

FIG. 25 provides viability data for SC-islets evaluated at 5 months (1 device) or 6 months (2 devices) post-implant into diabetic NSG mice. SC islets were generated using the regimen 2 or regimen 1 during stage 6 of differentiation. High viability is observed for devices with cells generated by either the regimen 2 or regimen 1 at stage 6 of differentiation.

FIG. 26 provides illustrative histology images of sections stained for C-peptide and glucagon (left panels) or H&E stained (right panels), 6 months after implant of SC-islets into diabetic NSG mice. These results show that SC-islets generated using compositions and methods of the disclosure, including regimen 2 and regimen 1, can remain viable after implantation, and are capable of producing insulin and glucagon.

### DETAILED DESCRIPTION OF THE DISCLOSURE

Pancreatic islet transplantation is a promising therapy that can achieve significant clinical benefit for diabetic subjects, for example, subjects with type I diabetes. As there is a limited supply of pancreatic islets sources from donor pancreatic tissue, there is a need for improved techniques to generate implantable islets from alternative sources, such as stem cells. Improved methods of generating islet components (e.g., SC-β cells) could result in more effective therapeutic products (e.g., SC-β cells with improved functionality), improved methods of manufacturing SC-islets for human therapeutic use (e.g., higher cell yields), or a combination thereof.

Provided herein are, inter alia, compositions and methods for improved production of SC-β cells in vitro. Certain compositions and combinations of agents disclosed here improve the fitness and metabolic flexibility of the differentiating cells and the resulting SC-β cells by targeting specific aspects of cellular metabolism. For example, the disclosure provides novel formulations and differentiation methods that result in higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, and improved viability, function, and reduced immunogenicity after transplantation. The disclosed compositions and methods can be employed in the large scale manufacture of SC-islets for human therapeutic use.

Metabolic pathways that can be targeted by compositions disclosed herein include, for example, one-carbon metabolism, acetyl-CoA synthesis for the generation of lipids and acetylation of proteins, fueling mitochondrial oxidative phosphorylation and TCA cycle, and generation of intermediates to maintain redox homeostasis. Compositions of the disclosure that are useful for improved production of SC-β cells can comprise, for example, a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, carnitine, or any combination thereof.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to is a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic F cells. Islet cells can also refer to a group of cells, cell clusters, or the like.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, when cultured under conditions suitable for differentiating the precursor cell into the insulin-positive endocrine cell.

The term “exocrine cell” as used herein can refer to a cell of an exocrine gland, i.e. a gland that discharges its secretion via a duct. In particular embodiments, an exocrine cell can refer to a pancreatic exocrine cell, which is a pancreatic cell that can produce enzymes that are secreted into the small intestine. These enzymes can help digest food as it passes through the gastrointestinal tract. Pancreatic exocrine cells are also known as islets of Langerhans, which can secrete two hormones, insulin and glucagon. A pancreatic exocrine cell can be one of several cell types; α-2 cells (which can produce the hormone glucagon); or β cells (which can manufacture the hormone insulin); and α-1 cells (which can produce the regulatory agent somatostatin). Non-insulin-producing exocrine cells, as the term is used herein, can refer to α-2 cells or α-1 cells. The term pancreatic exocrine cells encompasses “pancreatic endocrine cells” which can refer to a pancreatic cell that produces hormones (e.g., insulin (produced from β cells), glucagon (produced by alpha-2 cells), somatostatin (produced by delta cells) and pancreatic polypeptide (produced by F cells) that are secreted into the bloodstream.

The terms “stem cell-derived β cell,” “SC-β cell,” “functional β cell,” “functional pancreatic β cell,” “mature SC-β cell,” and their grammatical equivalents can refer to cells (e.g., non-native pancreatic β cells) that display at least one marker indicative of a pancreatic β cell (e.g., PDX-1 or NKX6.1), expresses insulin, and display a glucose stimulated insulin secretion (GSIS) response characteristic of an endogenous mature β cell. In some embodiments, the terms “SC-β cell” and “non-native β cell” as used herein are interchangeable. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. The GSIS response of the SC-3 cell can be observed within two weeks of transplantation of the SC-β cell into a host (e.g., a human or animal).

In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose.

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic 3-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive, endocrine cells or a precursor thereof into SC-β cells according to the methods of the present disclosure can be pancreatic β cell or (β-like cells (e.g., cells that have at least one, or at least two least two) characteristic of an endogenous β cell and exhibit a glucose stimulated insulin secretion (GSIS) response that resembles an endogenous adult β cell. The population of insulin-producing cells, e.g. produced by the methods as disclosed herein can comprise mature pancreatic 3 cell or SC-β cells, and can also contain non-insulin-producing cells (e.g., cells of cell like phenotype with the exception they do not produce or secrete insulin).

The terms “insulin-positive 1-like cell,” “insulin-positive endocrine cell,” and their grammatical equivalents can refer to cells (e.g., pancreatic endocrine cells) that displays at least one marker indicative of a pancreatic β cell and also expresses insulin but lack a glucose stimulated insulin secretion (GSIS) response characteristic of an endogenous β cell.

The term “β cell marker” refers to, without limitation, proteins, peptides, nucleic acids, polymorphism of proteins and nucleic acids, splice variants, fragments of proteins or nucleic acids, elements, and other analyte which are specifically expressed or present in pancreatic β cells. Exemplary β cell markers include, but are not limited to, pancreatic and duodenal homeobox 1 (Pdx1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, and MafA, and those described in Zhang et al., Diabetes. 50(10):2231-6 (2001). In some embodiment, the β cell marker is a nuclear 3-cell marker. In some embodiments, the β cell marker is Pdx1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analyte which are specifically expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g. β cells that produce insulin; a cells that produce glucagon; δ cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “Pdx1-positive pancreatic progenitor” as used herein can refer to a cell which is a pancreatic endoderm (PE) cell which has the capacity to differentiate into SC-β cells, such as pancreatic β cells. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. In some cases, a Pdx1-positive pancreatic progenitor cell lacks expression of NKX6.1. In some cases, a Pdx1-positive pancreatic progenitor cell can also be referred to as Pdx1-positive, NKX6.1-negative pancreatic progenitor cell due to its lack of expression of NKX6.1. In some cases, the Pdx1-positive pancreatic progenitor cells can also be termed as “pancreatic foregut endoderm cells.”

The term “Pdx1-positive, NKX6-1-positive pancreatic progenitor” as used herein can refer to a cell which is a pancreatic endoderm (PE) cell which has the capacity to differentiate into insulin-producing cells, such as pancreatic β cells. A Pdx1-positive, NKX6-1-positive pancreatic progenitor expresses the markers Pdx1 and NKX6-1. Other markers may include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR. As used herein, the terms “NKX6.1” and “NKX6-1” are equivalent and interchangeable. In some cases, the Pdx1-positive, NKX6-1-positive pancreatic progenitor cells can also be termed as “pancreatic foregut precursor cells.”

The term “Ngn3-positive endocrine progenitor” as used herein can refer to precursors of pancreatic endocrine cells expressing the transcription factor Neurogenin-3 (Ngn3). Progenitor cells are more differentiated than multipotent stem cells and can differentiate into only few cell types. In particular, Ngn3-positive endocrine progenitor cells have the ability to differentiate into the five pancreatic endocrine cell types (a, p, 6, F and PP). The expression of Ngn3 may be assessed by any method known by the skilled person such as immunochemistry using an anti-Ngn3 antibody or quantitative RT-PCR.

The terms “NeuroD” and “NeuroD1” are used interchangeably and identify a protein expressed in pancreatic endocrine progenitor cells and the gene encoding it.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. The term “selectable marker” as used herein can refer to a gene or to an expression product of the gene, e.g., an encoded protein. In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The term “epigenetics” refers to heritable changes in gene function that do not involve changes in the DNA sequence. Epigenetics most often denotes changes in a chromosome that affect gene activity and expression, but can also be used to describe any heritable phenotypic change that does not derive from a modification of the genome. Such effects on cellular and physiological phenotypic traits can result from external or environmental factors, or be part of normal developmental program. Epigenetics can also refer to functionally relevant changes to the genome that do not involve a change in the nucleotide sequence. Examples of mechanisms that produce such changes are DNA methylation and histone modification, each of which alters how genes are expressed without altering the underlying DNA sequence. Gene expression can be controlled through the action of repressor proteins that attach to silencer regions of the DNA. These epigenetic changes can last through cell divisions for the duration of the cell's life, and can also last for multiple generations even though they do not involve changes in the underlying DNA sequence of the organism. One example of an epigenetic change in eukaryotic biology is the process of cellular differentiation. During morphogenesis, totipotent stem cells become the various pluripotent cells, which in turn can become fully differentiated cells.

The term “epigenetic modifying compound” refers to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic modification has a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

The term “differentiated cell” or its grammatical equivalents is meant any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ˜98% of the cells become exocrine, ductular, or matrix cells, and ˜2% become endocrine cells. Early endocrine cells are islet progenitors, which can then differentiate further into insulin-producing cells (e.g. functional endocrine cells) which secrete insulin, glucagon, somatostatin, or pancreatic polypeptide. Endoderm cells can also be differentiate into other cells of endodermal origin, e.g. lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of the respiratory and digestive tracts (e.g. the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker Pdx1 (e.g. they are Pdx1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “subject,” “patient,” or “individual” are used interchangeably herein, and can refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject can refer to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The terms “treat,” “treating,” “treatment,” and their grammatical equivalents, as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” can refer to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “therapeutically effective amount”, therapeutic amount”, or its grammatical equivalents can refer to an amount effective, at dosages and for periods of time necessary, to achieve a desired therapeutic result. The therapeutically effective amount can vary according to factors such as the disease state, age, sex, and weight of the individual and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” e.g., the effect completely or partially prevents a disease or symptom thereof. A “prophylactically effective amount” can refer to an amount effective, at dosages and for periods of time necessary, to achieve a desired prophylactic result (e.g., prevention of disease onset).

Some numerical values disclosed throughout are referred to as, for example, “X is at least or at least about 100; or 200 [or any numerical number].” This numerical value includes the number itself and all of the following:
a. X is at least 100;
b. X is at least 200;
c. X is at least about 100; and
d. X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i) X being administered on between day 1 and day 2;
ii) X being administered on between day 2 and day 3;
iii) X being administered on between about day 1 and day 2;
iv) X being administered on between about day 2 and day 3;
v) X being administered on between day 1 and about day 2;
vi) X being administered on between day 2 and about day 3;
vii) X being administered on between about day 1 and about day 2; and
viii) X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

### Stages of Differentiation

In some embodiments, pancreatic differentiation as disclosed herein is carried out in a step-wise manner. In the step-wise progression, “Stage 1” or “S1” refers to the first step in the differentiation process, the differentiation of pluripotent stem cells into cells expressing markers characteristic of definitive endoderm cells (“DE”, “Stage β cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor β cells (“PP1”, “Stage 3 cells” or “S3 cells”). “Stage 4” refers to the fourth step, the differentiation of cells expressing markers characteristic of pancreatic progenitor β cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells (e.g., PDX.1⁺, NKX6.1⁺) into cells expressing markers characteristic of pancreatic endoderm cells and/or pancreatic endocrine progenitor cells (e.g., insulin+) (“EN”, “Stage 5 cells” or “S5 cells”). “Stage 6” refers to the differentiation of cells expressing markers characteristic of pancreatic endocrine progenitor cells (e.g., insulin) into cells expressing markers characteristic of pancreatic endocrine β cells (“SC-β cells”) or pancreatic endocrine α cells (“SC-α cells”). It should be appreciated, however, that not all cells in a particular population progress through these stages at the same rate, i.e., some cells may have progressed less, or more, down the differentiation pathway than the majority of cells present in the population. For example, in some embodiments, SC-β cells can be identified during stage 5, at the conclusion of stage 5, at the beginning of stage 6, etc. Examples of methods of making cells of any one of stages 1-6 are provided in, for example, U.S. Pat. Nos. 10,030,229; 10,443,042; published application US 20200332262; and pending US application U.S. Ser. No. 17/010,346, each of which is incorporated by reference in its entirety.

### Culture Medium and Agents

### Acetyl CoA-Related Metabolite

In some embodiments, a composition (e.g., medium) of the disclosure comprises an acetyl CoA-related metabolite. Metabolism of acetyl-coenzyme A (acetyl-CoA) can confer numerous metabolic functions, including energy production, lipid synthesis, and protein acetylation.

In some embodiments, contacting cells with an acetyl-coenzyme A-related metabolite (e.g., acetate) can improve production of SC-β cells in vitro, for example, higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, for example, compared to a composition that lacks the acetyl-coenzyme A-related metabolite or contains it at a lower concentration.

Exemplary acetyl CoA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl CoA-related metabolite is acetate. In some embodiments, a composition of the disclosure contains two or more different acetyl CoA related metabolites, for example, 2, 3, 4, 5, 6, 7, 8, 9, 10, or more different acetyl CoA-related metabolites. In some embodiments, the acetyl-CoA-related metabolite is acetate.

In some embodiments, the acetyl CoA-related metabolite is present in or is added to a composition of the disclosure at a concentration of at least 0.1 nM, at least 1 nM, at least 10 nM, at least 50 nM, at least 80 nM, at least 100 nM, at least 120 nM, at least 140 nM, at least 150 nM, at least 200 nM, at least 300 nM, at least 500 nM, at least 800 nM, at least 1 μM, at least 10 μM, at least 100 μM, at least 500 μM, at least 800 μM, at least 900 μM, at least 1 mM, at least 2 mM, at least 3 mM, at least 5 mM, or at least 10 mM.

In some embodiments, the acetyl CoA-related metabolite is present in or is added to a composition of the disclosure at a concentration of at most 200 nM, at most 300 nM, at most 500 nM, at most 800 nM, at most 1 μM, at most 10 μM, at most 100 μM, at most 500 μM, at most 800 μM, at most 900 μM, at most 1 mM, at most 2 mM, at most 3 mM, at most 5 mM, or at most 10 mM.

In some embodiments, the acetyl CoA-related metabolite is present in or is added to a composition of the disclosure at a concentration of about 10 nM, about 50 nM, about 80 nM, about 100 nM, about 120 nM, about 140 nM, about 150 nM, about 200 nM, about 300 nM, about 500 nM, about 800 nM, about 1 μM, about 10 μM, about 100 μM, about 500 μM, about 800 μM, about 900 μM, about 1 mM, about 2 mM, about 3 mM, about 5 mM, or about 10 mM.

In some embodiments, the acetyl CoA-related metabolite is present in or is added to a composition of the disclosure at a concentration of about 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM. In some embodiments, the acetyl CoA-related metabolite is acetate present at a concentration of about 1 mM.

In some embodiments, the acetyl CoA-related metabolite is acetate present at a concentration of about 50-1000 nM, 50-800 nM, 50-500 nM, 50-300 nM, 50-250 nM, 100-200 nM, or 125-175 nM. In some embodiments, the acetyl CoA-related metabolite is acetate present at a concentration of about 160 nM.

In some embodiments, cells are contacted with the acetyl CoA-related metabolite (e.g., acetate) in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the acetyl CoA-related metabolite (e.g., acetate) for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the acetyl CoA-related metabolite (e.g., acetate) for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the acetyl CoA-related metabolite (e.g., acetate) for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the acetyl CoA-related metabolite (e.g., acetate) for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include an acetyl CoA-related metabolite (e.g., does not include acetate).

### Vitamins

In some embodiments, a composition (e.g., medium) of the disclosure comprises one or more vitamins. In some embodiments, contacting cells with a vitamin (e.g., biotin) can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that does not contain the vitamin, or contains it at a lower concentration.

Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a co-factor for pyruvate carboxylase. In some embodiments, the vitamin is biotin. In some embodiments, a composition of the disclosure contains two or more different vitamins, for example, 2, 3, 4, 5, 6, 7, 8, 9, 10, or more different vitamins.

In some embodiments, the vitamin is present in or is added to a composition of the disclosure at a concentration of at least 0.1 nM, at least 1 nM, at least 10 nM, at least 50 nM, at least 80 nM, at least 100 nM, at least 120 nM, at least 140 nM, at least 150 nM, at least 200 nM, at least 300 nM, at least 500 nM, at least 600 nM, at least 700 nM, at least 800 nM, at least 900 nM, at least 1 μM, at least 10 μM, at least 100 μM, at least 500 μM, at least 800 μM, at least 900 μM, at least 1 mM, at least 2 mM, at least 3 mM, at least 5 mM, or at least 10 mM.

In some embodiments, the vitamin is present in or is added to a composition of the disclosure at a concentration of at most 200 nM, at most 300 nM, at most 500 nM, at most 600 nM, at most 700 nM, at most 800 nM, at most 900 nM, at most 1 μM, at most 1.5 μM, at most 3 μM, at most 5 μM, at most 10 μM, at most 100 μM, at most 500 μM, at most 800 μM, at most 1 mM, or at most 10 mM.

In some embodiments, the vitamin is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the vitamin is biotin present at a concentration of about 800 nM.

In some embodiments, the vitamin is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, cells are contacted with the vitamin (e.g., biotin) in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the vitamin (e.g., biotin) for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the vitamin (e.g., biotin) for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the vitamin (e.g., biotin) for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the vitamin (e.g., biotin) for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include a vitamin (e.g., does not include biotin).

### Histone Deacetylase Inhibitor (HDACi)

In some embodiments, a composition (e.g., medium) of the disclosure comprises a histone deacetylase inhibitor (HDACi). Histone deacetylase inhibitors (HDACi) are a class of compounds that increase acetylation of lysine residues on histone proteins as well as other, nonhistone, proteins by inhibiting the activity of HDAC enzymes.

In some embodiments, contacting cells with an HDACi can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks the HDACi, or contains it at a lower concentration.

Exemplary histone deacetylase inhibitors (HDACi) include, but are not limited to (3-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abexinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (CI994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UF010, Tasquinimod (ABR-215050), SKLB-23bb, Isoguanosine, Sulforaphane, BRD73954, Citarinostat (ACY-241, HDAC-IN-2), Suberohydroxamic acid, Splitomicin, HPOB, LMK-235, Biphenyl-4-sulfonyl chloride (p-Phenylbenzenesulfonyl, 4-Phenylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, Raddeanin A, Tinostamustine(EDO-S101), Domatinostat (4SC-202), and BG45.

In some embodiments, the HDACi is β-Hydroxybutyrate. β-Hydroxybutyric acid is a ketone body that, along with butyric acid, is an agonist of hydroxycarboxylic acid receptor 2 (HCA2), a Gi/o-coupled GPCR. In some embodiments, an HDACi inhibitor is an agonist of hydroxycarboxylic acid receptor 2.

In some embodiments, a composition of the disclosure contains two or more different HDACi, for example, 2, 3, 4, 5, 6, 7, 8, 9, 10, or more different HDACi.

In some embodiments, the HDACi is present in or is added to a composition of the disclosure at a concentration of at least 0.1 nM, at least 1 nM, at least 10 nM, at least 50 nM, at least 80 nM, at least 100 nM, at least 120 nM, at least 140 nM, at least 150 nM, at least 200 nM, at least 300 nM, at least 500 nM, at least 600 nM, at least 700 nM, at least 800 nM, at least 900 nM, at least 1 μM, at least 1.2 μM, at least 1.5 μM, at least 1.8 μM, at least 2 μM, at least 3 μM, at least 5 μM, at least 10 μM, at least 100 μM, at least 500 μM, at least 800 μM, at least 900 μM, at least 1 mM, at least 2 mM, at least 3 mM, at least 5 mM, or at least 10 mM.

In some embodiments, the HDACi is present in or is added to a composition of the disclosure at a concentration of at most 150 nM, at most 200 nM, at most 300 nM, at most 500 nM, at most 600 nM, at most 700 nM, at most 800 nM, at most 900 nM, at most 1 μM, at most 1.2 μM, at most 1.5 μM, at most 1.8 μM, at most 2 μM, at most 3 μM, at most 5 μM, at most 10 μM, at most 100 μM, at most 500 μM, at most 800 μM, at most 1 mM, or at most 10 mM.

In some embodiments, the HDACi is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the HDACi is β-Hydroxybutyrate present at a concentration of about 200 nM.

In some embodiments, the HDACi is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, cells are contacted with the HDACi (e.g., β-Hydroxybutyrate) in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the HDACi (e.g., β-Hydroxybutyrate) for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the HDACi (e.g., β-Hydroxybutyrate) for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the HDACi (e.g., β-Hydroxybutyrate) for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the HDACi (e.g., β-Hydroxybutyrate) for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include an HDACi (e.g., does not include β-Hydroxybutyrate)

### Redox Homeostasis Regulator

In some embodiments, a composition (e.g., medium) of the disclosure comprises a redox homeostasis regulator. Redox homoeostasis inhibitors can, for example, improve viability and cell function by minimizing oxidative damage to cells, or promote appropriate oxidation of substrates as part of biological processes.

In some embodiments, contacting cells a redox homeostasis regulator (e.g., taurine) can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks the redox homeostasis regulator, or contains it at a lower concentration.

Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10.

In some embodiments, the redox homeostasis regulator is taurine.

Taurine is a non-proteinogenic β-aminosulfonic acid that can be derived from methionine and cysteine metabolism. In some embodiments, taurine can inhibit ROS generation within the respiratory chain.

In some embodiments, a composition of the disclosure contains two or more different redox homeostasis regulators, for example, 2, 3, 4, 5, 6, 7, 8, 9, 10, or more different redox homeostasis regulators.

In some embodiments, the redox homeostasis regulator is present in or is added to a composition of the disclosure at a concentration of at least 100 nM, at least 500 nM, 1 μM, at least 10 μM, at least 20 μM, at least 30 μM, at least 40 μM, at least 50 μM, at least 60 μM, at least 70 μM, at least 80 μM, at least 90 μM, at least 100 μM, at least 110 μM, at least 110 μM, at least 150 μM, at least 200 μM, or at least 500 μM.

In some embodiments, the redox homeostasis regulator is present in or is added to a composition of the disclosure at a concentration of at most 10 μM, at most 20 μM, at most 30 μM, at most 40 μM, at most 50 μM, at most 60 μM, at most 70 μM, at most 80 μM, at most 90 μM, at most 100 μM, at most 110 μM, at most 110 μM, at most 150 μM, at most 200 μM, or at most 500 μM.

In some embodiments, the redox homeostasis regulator is present in or is added to a composition of the disclosure at a concentration of about 100 nM, about 500 nM, 1 μM, about 10 μM, about 20 μM, about 30 μM, about 40 μM, about 50 μM, about 60 μM, about 70 μM, about 80 μM, about 90 μM, about 100 μM, about 110 μM, about 110 μM, about 150 μM, or about 200 μM. In some embodiments, the redox homeostasis regulator is taurine. In some embodiments, the redox homeostasis regulator is taurine present at a concentration of about 90 μM.

In some embodiments, the redox homeostasis regulator intermediate is present or is added at a concentration of about 100 nM to 1 mM, 500 nM to 1 mM, 1 μM to 1 mM, 10 μM to 1 mM, 20 μM to 1 mM, 30 μM to 1 mM, 30 μM to 1 mM, 40 μM to 1 mM, 50 μM to 1 mM, 60 μM to 1 mM, 70 μM to 1 mM, 80 μM to 1 mM, 100 nM to 250 μM, 500 nM to 250 μM, 1 μM to 250 μM, 10 μM to 250 μM, 20 μM to 250 μM, 30 μM to 250 μM, 30 μM to 250 μM, 40 μM to 250 μM, 50 μM to 250 μM, 60 μM to 250 μM, 70 μM to 250 μM, 100 nM to 100 μM, 500 nM to 100 μM, 1 μM to 100 μM, 10 μM to 100 μM, 20 μM to 100 μM, 30 μM to 100 μM, 40 μM to 100 μM, 50 μM to 100 μM, 60 μM to 100 μM, 70 μM to 100 μM, or 80 μM to 100 μM.

In some embodiments, cells are contacted with the redox homeostasis regulator (e.g., taurine) in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the redox homeostasis regulator (e.g., taurine) for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the redox homeostasis regulator (e.g., taurine) for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the redox homeostasis regulator (e.g., taurine) for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the redox homeostasis regulator (e.g., taurine) for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include a redox homeostasis regulator (e.g., does not include taurine).

### One Carbon Metabolism Pathway Intermediate

In some embodiments, a composition (e.g., medium) of the disclosure comprises a one carbon metabolism pathway intermediate. One-carbon metabolism mediated by folate cofactors, supports multiple physiological processes including amino acid homeostasis (methionine, glycine and serine), biosynthesis of nucleotides (purines, thymidine), epigenetic maintenance, and redox defense.

In some embodiments, contacting cells with a one carbon metabolism pathway intermediate can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks the one carbon metabolism pathway intermediate, or contains it at a lower concentration.

Exemplary one carbon metabolism pathway intermediates include, but are not limited to formate, tetrahydrofolate (THF), 10-formylTHF; 5,10-meTHF; 5,10-meTHF; and 10-formylTHF.

In some embodiments, a composition of the disclosure contains two or more different one carbon metabolism pathway intermediates, for example, 2, 3, 4, 5, 6, 7, 8, 9, 10, or more different one carbon metabolism pathway intermediates.

In some embodiments, the one carbon metabolism pathway intermediate is present in or is added to a composition of the disclosure at a concentration of at least 100 nM, at least 500 nM, at least 1 μM, at least 10 μM, at least 20 μM, at least 30 μM, at least 40 μM, at least 50 μM, at least 75 μM, or at least 100 μM.

In some embodiments, the one carbon metabolism pathway intermediate is present in or is added to a composition of the disclosure at a concentration of at most 10 μM, at most 20 μM, at most 30 μM, at most 40 μM, at most 50 μM, at most 75 μM, at most 100 μM, at most 200 μM, at most 300 μM, at most 400 μM, at most 500 μM, at most 1 mM, or at most 10 mM.

In some embodiments, the one carbon metabolism pathway intermediate is present in or is added to a composition of the disclosure at a concentration of about 100 nM, about 500 nM, 1 μM, about 10 μM, about 20 μM, about 30 μM, about 40 μM, about 50 μM, about 75 μM, or about 100 μM.

In some embodiments, the one carbon metabolism pathway intermediate is formate present at a concentration of about 50 μM.

In some embodiments, the one carbon metabolism pathway intermediate is present or is added at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 40 μM to 60 μM, or 45 μM to 55 μM.

In some embodiments, cells are contacted with the one carbon metabolism pathway intermediate (e.g., formate) in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the one carbon metabolism pathway intermediate (e.g., formate) for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the one carbon metabolism pathway intermediate (e.g., formate) for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the one carbon metabolism pathway intermediate (e.g., formate) for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the one carbon metabolism pathway intermediate (e.g., formate) for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include a one carbon metabolism pathway intermediate (e.g., does not include formate).

### Glutamine

In some embodiments, a composition (e.g., medium) of the disclosure comprises glutamine.

In some embodiments, contacting cells with glutamine as disclosed herein can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks glutamine, contains less glutamine, or contains glutamine in a different (e.g., less bioavailable) form.

Glutamine (Gln or Q) is an alpha-amino acid. Glutamine can be an essential amino acid within in vitro cell cultures. Glutamine supports the growth of cells, including cells that have high energy demands and synthesize large amounts of proteins and nucleic acids. It is an alternative energy source for rapidly dividing cells and cells that use glucose inefficiently.

In some embodiments, compositions and methods of the disclosure utilize glutamine in a form with increased bioavailability. Because of its chemical instability and importance for cell growth and function, it is important that delivery of L-glutamine be tailored to each unique cell culture process. Glutamine (e.g., L-glutamine) in a free form can be unstable at physiological pH in liquid media, breaking down to ammonium and pyroglutamate at rates that make it a problem in many cell culture and biomanufacturing applications. Therefore, many cell culture media contain stabilized forms of glutamine, including dipeptide forms, such as alanyl−1-glutamine and glycyl−1-glutamine. However, these more stable forms of L-glutamine can also have limited bioavailability, for example, due to a requirement for processing by enzymes, such as cell surface peptidases. Thus in some embodiments, compositions and methods of the disclosure utilize glutamine in a form with increased bioavailability, such as a free glutamine form, such as a non-dipeptide form, a non-alanine-glutamine dipeptide form (e.g., a non-alanyl−1-glutamine form), a non-glycine-glutamine dipeptide form (e.g., a non-glycyl−1-glutamine form), a form that in which glutamine is not conjugated to another amino acid or stabilizing moiety, a monomeric form, a free form, or a combination thereof. In some embodiments, glutamine is provided as a protein hydrolysate.

In some embodiments, a basal media contains glutamine. In some embodiments, glutamine in a form as disclosed herein is added to a media that already contains glutamine. In some embodiments, glutamine in a form as disclosed herein is added to a basal media that contains no glutamine or only low levels of glutamine to increase the bioavailability of glutamine.

Glutamine can contribute to multiple cellular metabolic processes that are important to aspects of the disclosure, for example, redox regulation, NAD recycling, TCA cycle anaplerosis, and nitrogen metabolism. Glutamine contains one atom of nitrogen as an amide and another atom of nitrogen as an amine and it transports and delivers nitrogen to cells in quantities that are toxic as free ammonium. Glutamine amide nitrogen can be used in the synthesis of NAD and NADP, purine nucleotides, CTP from UTP, and asparagine. Nitrogen initially stored in glutamine can also be used to produce carbamyl phosphate for the synthesis of pyrimidines. Glutamine is a precursor of glutamate, a key amino acid used for the transamination of alpha ketoacids to form other alpha amino acids. When glucose levels are low and energy demands are high, cells can metabolize amino acids for energy. Glutamine is one of the most readily available amino acids for use as an energy source and it is a major source of energy for many rapidly dividing cell types in vitro.

Cells require nitrogen atoms to build molecules such as nucleotides, amino acids, amino-sugars and vitamins. Ammonium is an inorganic source of nitrogen that exists primarily as a positively charged cation, NH4+, at physiological pH. Ammonium nitrogen used by cells is initially incorporated into organic nitrogen as an amine of glutamate or an amide of glutamine. These two amino acids provide the primary reservoirs of nitrogen for the synthesis of proteins, nucleic acids and other nitrogenous compounds. Reactions that fix nitrogen into glutamate and glutamine consume energy equivalents. Glutamate can be synthesized from ammonium and alpha ketoglutaric acid, a tricarboxylic acid (TCA) cycle intermediate. Its synthesis can require the oxidation of either NADH or NADPH. Glutamine is formed from ammonium and glutamate and its synthesis consumes ATP. The enzymes involved in glutamate synthesis, glutamate dehydrogenase (EC 1.4.1.4) and glutamate synthase (EC 1.4.1.13) are reversible. The enzyme responsible for glutamine synthesis, glutamine synthetase (EC 6.3.1.2), is highly regulated to limit the production of glutamine to cell requirements. The catabolism of glutamine to glutamate and ammonium is mediated by mitochondrial enzymes called glutaminases (EC 3.5.1.2). Ammonium produced in vivo can be metabolized to urea. In vitro, ammonium is not metabolized to urea. Under some in vitro conditions, ammonia accumulates in the extracellular medium as ammonium ion.

Cellular demands for glutamine can be increased by stressful conditions. In some embodiments, glutamine is a conditionally-essential amino acid, e.g., must be provided at a sufficient level in conditions of stress. Like other amino acids, glutamine is biochemically important as a constituent of proteins. Glutamine can also be crucial in nitrogen metabolism. Ammonia (formed by nitrogen fixation) can be assimilated into organic compounds by converting glutamic acid to glutamine. The enzyme which accomplishes this is called glutamine synthetase. Glutamine can be used as a nitrogen donor in the biosynthesis of many compounds, including other amino acids, purines, and pyrimidines. L-glutamine can improve nicotinamide adenine dinucleotide (NAD) redox potential.

In some embodiments, glutamine can be present in a composition of the disclosure at a concentration of at least 100 μM, at least 250 μM, at least 500 μM, at least 750 μM, at least 1 mM, at least 1.5 mM, at least 2 mM, at least 2.5 mM, at least 2.6 mM, at least 2.7 mM, at least 2.8 mM, at least 2.9 mM, at least 3 mM, at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, or at least 5 mM.

In some embodiments, glutamine can be present in a composition of the disclosure at a concentration of at most 2 mM, at most 3 mM, at most 3.1 mM, at most 3.2 mM, at most 3.3 mM, at most 3.4 mM, at most 3.5 mM, at most 3.6 mM, at most 3.7 mM, at most 3.8 mM, at most 3.9 mM, at most 4 mM, at most 4.1 mM, at most 4.2 mM, at most 4.2 mM, at most 4.4 mM, at most 4.5 mM, at most 4.75 mM, at most 5 mM, at most 6 mM, at most 7 mM, at most 8 mM, at most 9 mM, at most 10 mM, at most 15 mM, at most 20 mM, at most 30 mM, at most 40 mM, or at most 50 mM.

In some embodiments, glutamine can be present in a composition of the disclosure at a concentration of about 100 μM, about 250 μM, about 500 μM, about 750 μM, about 1 mM, about 1.5 mM, about 2 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, about 3.7 mM, about 3.8 mM, about 3.9 mM, about 4 mM, or about 5 mM.

In some embodiments, glutamine is present or is added to a composition of the disclosure at a concentration of from 0.5-20 mM, 0.5-10 mM, 0.5-5 mM, 1-5 mM, 2-5 mM, or 1 mM to 10 mM. In some embodiments, glutamine is present or is added to a composition of the disclosure at a concentration of 3.8-4.2 mM. In some embodiments, glutamine is present or is added to a composition of the disclosure at a concentration of 1-10, 1-7, 1-8, 1-6, 1-5, 1-4, 2-10, 2-7, 2-8, 2-6, 2-5, 2-4, 3-10, 3-7, 3-8, 3-6, 3-5, 3-4, 3.5-4.5, 3.8-4.2, or 3.9-4.1 mM. In some embodiments, glutamine is present or is added to a composition of the disclosure at a concentration of about 4 mM.

In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a dipeptide form.

In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in an alanine-glutamine dipeptide form.

In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a glycine-glutamine dipeptide form.

In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a multimeric form.

In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not conjugated to another amino acid or stabilizing moiety.

In some embodiments, at least 500 μM, at least 750 μM, at least 1 mM, at least 1.5 mM, at least 2 mM, at least 2.5 mM, at least 2.6 mM, at least 2.7 mM, at least 2.8 mM, at least 2.9 mM, at least 3 mM, at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, or at least 5 mM of the glutamine is in a monomeric form.

In some embodiments, at least 500 μM, at least 750 μM, at least 1 mM, at least 1.5 mM, at least 2 mM, at least 2.5 mM, at least 2.6 mM, at least 2.7 mM, at least 2.8 mM, at least 2.9 mM, at least 3 mM, at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, at least 5 mM, at least 5.5 mM, at least 6 mM, at least 6.5 mM, at least 7 mM, at least 7.5 mM, at least 8 mM, at least 8.5 mM, at least 9 mM, at least 9.5 mM, or at least 10 mM of the glutamine is in a free form.

In some embodiments, cells are contacted with glutamine in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with glutamine for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the glutamine for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the glutamine for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the glutamine for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include glutamine.

### Glutamate

In some embodiments, a composition (e.g., medium) of the disclosure comprises glutamate (e.g., L-glutamate). Glutamate can be converted into, for example, g-amino butyric acid (GABA), ornithine, 2-oxoglutarate, glucose or glutathione. Glutamate and metabolites generated therefrom can contribute to, for example, redox homeostasis, cell signaling, nitrogen assimilation, amine catabolism, amino acid biosynthesis, nucleoside biosynthesis, and cofactor production.

In some embodiments, contacting cells with glutamate can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks glutamate, or contains a lower concentration of glutamate.

In some embodiments, glutamate can be present in a composition of the disclosure at a concentration of at least 100 μM, at least 200 μM, at least 300 μM, at least 400 μM, at least 450 μM, at least 500 μM, at least 600 μM, at least 700 μM, at least 800 μM, at least 900 μM, at least 1 mM, at least 1.5 mM, at least 2 mM, at least 2.5 mM, at least 3 mM, at least 4 mM, or at least 5 mM.

In some embodiments, glutamate can be present in a composition of the disclosure at a concentration of at most 500 μM, at most 600 μM, at most 700 μM, at most 800 μM, at most 900 μM, at most 1 mM, at most 2 mM, at most 3 mM, at most 4 mM, at most 5 mM, at most 6 mM, at most 7 mM, at most 8 mM, at most 9 mM, at most 10 mM, at most 15 mM, at most 20 mM, at most 30 mM, at most 40 mM, or at most 50 mM.

In some embodiments, glutamate can be present in a composition of the disclosure at a concentration of about 100 μM, about 200 μM, about 300 μM, about 400 μM, about 450 μM, about 500 μM, about 550 μM, about 600 μM, about 700 μM, about 800 μM, about 900 μM, about 1 mM, about 1.5 mM, about 2 mM, about 2.5 mM, about 3 mM, about 4 mM, or about 5 mM. In some embodiments, glutamate is present or is added to a composition of the disclosure at a concentration of about 500 μM.

In some embodiments, glutamate is present or is added to a composition of the disclosure at a concentration of from about 100 μM to 5 mM, 200 μM to 5 mM, 300 μM to 5 mM, 400 μM to 5 mM, 100 μM to 3 mM, 200 μM to 3 mM, 300 μM to 3 mM, 400 μM to 3 mM, 100 μM to 2 mM, 200 μM to 2 mM, 300 μM to 2 mM, 400 μM to 2 mM, 100 μM to 1 mM, 200 μM to 1 mM, 300 μM to 1 mM, 400 μM to 1 mM, 100 μM to 700 μM, 200 μM to 700 μM, 300 μM to 700 μM,

400 μM to 700 μM, 100 μM to 600 μM, 200 μM to 600 μM, 300 μM to 600 μM, or 400 μM to 600 μM.

In some embodiments, cells are contacted with glutamate in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the glutamate for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the glutamate for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the glutamate for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the glutamate for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include glutamate.

### Carnitine

In some embodiments, a composition (e.g., medium) of the disclosure comprises carnitine. Carnitine transports long-chain fatty acids into mitochondria to be oxidized for energy production, and can support cell fitness.

In some embodiments, contacting cells with carnitine can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks carnitine, or contains a lower concentration of carnitine.

In some embodiments, carnitine is present in or is added to a composition of the disclosure at a concentration of at least 100 nM, at least 500 nM, at least 1 μM, at least 10 μM, at least 15 μM, at least 20 μM, at least 25 μM, at least 30 μM, at least 35 μM, at least 40 μM, at least 50 μM, at least 75 μM, or at least 100 μM.

In some embodiments, carnitine is present in or is added to a composition of the disclosure at a concentration of at most 20 μM, at most 30 μM, at most 40 μM, at most 50 μM, at most 60 μM, 70 μM, at most 80 μM, at most 90 μM, at most 100 μM, at most 200 μM, at most 300 μM, at most 400 μM, at most 500 μM, or at most 1 mM.

In some embodiments, carnitine is present in or is added to a composition of the disclosure at a concentration of about 100 nM, about 500 nM, about 1 μM, about 10 μM, about 15 μM, about 20 μM, about 25 μM, about 30 μM, about 35 μM, about 40 μM, about 45 μM, about 50 μM, about 55 μM, about 60 μM, about 75 μM, or about 100 μM. In some embodiments, carnitine is present or is added at a concentration of about 40 μM.

In some embodiments, carnitine is present in or is added to a composition of the disclosure at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 35 μM to 60 μM, or 30 μM to 50 μM.

In some embodiments, cells are contacted with carnitine in methods of the disclosure. The cells can be PDX1-positive, NKX6.1-positive, insulin-negative cells. The cells can be insulin-positive endocrine progenitor cells. The cells can be SC-β cells. In some embodiments, the cells are frozen. In some embodiments, the cells have been previously frozen. In some embodiments, the cells are in cell clusters. In some embodiments, the cells are dissociated.

In some embodiments, cells (e.g., PDX1-positive, NKX6.1-positive, insulin-negative cells, or insulin-positive endocrine progenitor cells) are contacted with the carnitine for about 12 hours, 24 hours, 36 hours, 48 hours, 60 hours, 72 hours, 84 hours, 4 days, 5, days, 6 days, 7 days, 8 days, 9 days, 10 days, 11 days, 12 days, 13 days, or 14 days.

In some embodiments, cells (e.g., PDX1-positive, NKX6.1-positive, insulin-negative cells, or insulin-positive endocrine progenitor cells) are contacted with the carnitine for at least 12 hours, at least 24 hours, at least 36 hours, at least 48 hours, at least 60 hours, at least 72 hours, at least 84 hours, at least 4 days, at least 5, at least days, at least 6 days, at least 7 days, at least 8 days, at least 9 days, at least 10 days, at least 11 days, at least 12 days, at least 13 days, at least or at least 14 days.

In some embodiments, cells (e.g., PDX1-positive, NKX6.1-positive, insulin-negative cells, or insulin-positive endocrine progenitor cells) are contacted with the carnitine for at most 12 hours, at most 24 hours, at most 36 hours, at most 48 hours, at most 60 hours, at most 72 hours, at most 84 hours, at most 4 days, at most 5, at most days, at most 6 days, at most 7 days, at most 8 days, at most 9 days, at most 10 days, at most 11 days, at most 12 days, at most 13 days, at most or at most 14 days.

In some embodiments, cells (e.g., PDX1-positive, NKX6.1-positive, insulin-negative cells, or insulin-positive endocrine progenitor cells) are contacted with the carnitine for about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days.

In some embodiments, a composition or method of the disclosure does not include carnitine.

### TGF-β Signaling Pathway Inhibitor

Exemplary TGF-β signaling pathway inhibitors include, without limitation, ALK5 inhibitor II (CAS 446859-33-2, an ATP-competitive inhibitor of TGF-B Ri kinase, also known as RepSox, IIJPAC Name: 2-[5-(6-methylpyridin-2-yl)-1H-pyrazol-4-yl]-1,5-naphthyridine, an analog or derivative of ALK5 inhibitor II, such as an analog or derivative of ALK5 inhibitor II described in U.S. Pub. No. 2012/0021519, a TGF-β receptor inhibitor described in U.S. Pub. No. 2010/0267731, an ALK5 inhibitor described in U.S. Pub Nos. 2009/0186076 and 2007/0142376, including e.g., A83-01, 431542, D4476, GW788388, LY364947, LY580276, SB525334, SB505124, SD208, GW6604, or GW788388.

In some embodiments, the TGF-β signaling pathway inhibitor can have the following structure.

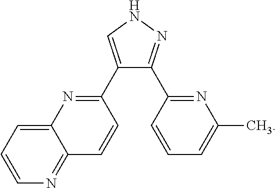 [SMILES: Cc1cccc(-c2n[nH]cc2-c2ccc3ncccc3n2)n1]

In some embodiments, the concentration of the TGF-β signaling pathway inhibitor can be from about 0.1-110 μM, 0.1-50 μM, 0.1-25 μM, or 0.1-10 μM. In some embodiments, the concentration of the TGF-β signaling pathway inhibitor can be about 10 μM. In some embodiments, the TGF-β signaling pathway inhibitor is an Alk5 inhibitor II and concentration of the inhibitor is about 10 μM.

### Thyroid Hormone Signaling Pathway Activator

Exemplary thyroid hormone signaling pathway activators include, without limitation, triiodothyronine (T3), an analog or derivative of T3, for example, selective and non-selective thyromimetics, TRJ selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-21 15 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine [T4] and triiodothyronine [T3] alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3 ^{f}-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA).

In some embodiments, the thyroid hormone signaling pathway activator can comprise a prodrug or prohormone of T3, such as T4 thyroid hormone (e.g., thyroxine or L-3,5,3′,5′-tetraiodothyronine). In some embodiments, the thyroid hormone signaling pathway activator can be an iodothyronine composition described in U.S. Pat. No. 7,163,918. In some embodiments, the thyroid hormone signaling pathway activator can be 2-[4-[[4-Hydroxy-3-(1-methylethyl)phenyl]methyl]-3,5-dimethylphenoxy]acetic acid (GC-1). GC-1 is a thyromimetic, high affinity agonist at thyroid hormone receptor (TR) β and TRoc receptors (K_{D} values are 67 and 440 p respectively). GC-1 displays 5- and 100-fold greater potency than the endogenous agonist T3 in vitro at TRoti and TR i receptors respectively.

In some embodiments, the thyroid hormone signaling pathway activators can have the following structure:

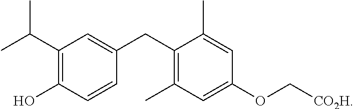 [SMILES: Cc1cc(OCC(=O)O)cc(C)c1Cc1ccc(O)c(C(C)C)c1]

In some embodiments, the concentration of the thyroid hormone signaling pathway activator can be from about 0.1-110 μM, 0.1-50 μM, 0.1-25 μM, or 0.1-10 μM. In some embodiments, the concentration of the thyroid hormone signaling pathway activator can be about 1 μM. In some embodiments, the thyroid hormone signaling pathway activator is GC-1 and the concentration of the activator is about 1 μM.

### Protein Kinase Inhibitor

Exemplary protein kinase inhibitors include, without limitation, staurosporine, an analog of staurosporine, such as Ro-31-8220, a bisindolylmaleimide (Bis) compound, 10′-{5″-[(methoxycarbonyl)amino]-2″-methyl}-phenylaminocarbonylstaurosporine, a staralog (see, e.g., Lopez et al., “Staurosporine-derived inhibitors broaden the scope of analog-sensitive kinase technology”, J. Am. Chem. Soc. 2013; i 35(48): 18153-18159), and cgp41251. In some embodiments, the protein kinase inhibitor can be staurosporine.

In some embodiments, the concentration of the protein kinase inhibitor can be from about 0.1-110 nM, 0.1-50 nM, 0.1-25 nM, 0.1-10 nM, or 0.1-5 nM. In some embodiments, the concentration of the protein kinase inhibitor can be about 3 nM. In some embodiments, the protein kinase inhibitor is staurosporine (SSP) and the concentration of the activator is about 3 nM.

In some embodiments, the protein kinase inhibitor can have the following structure:

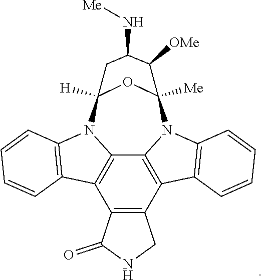 [SMILES: CN[C@@H]1C[C@H]2O[C@@](C)([C@@H]1OC)n1c3ccccc3c3c4c(c5c6ccccc6n2c5c31)C(=O)NC4]

### Bone Morphogenic Protein (BMP) Signaling Pathway Inhibitor

Exemplary BMP signaling pathway inhibitors include, without limitation, 4-[6-(4-piperazin-1-ylphenyl)pyrazol o [1,5-a]pyrimidin-3-yl] quinolone (LDN 193 189; also known as LDN1931 89, 1062368-24-4, LDN-193189, DM 3189, DM-3189, and referred to herein as LDN), an analog or derivative of LDN193189, e.g., a salt (e.g., LDN193189 hydrochloride), hydrate, solvent, ester, or prodrug of LDN193189, or a compound of Formula I from U.S. Patent Publication No. 2011/0053930. In accordance with aspects of the present invention, the BMP signaling pathway inhibitor comprises LDN193189. In some embodiments, the BMP signaling pathway inhibitor comprises DMH-1 or an analog or derivative thereof.

In some embodiments, the BMP signaling pathway inhibitor can have the following structure.

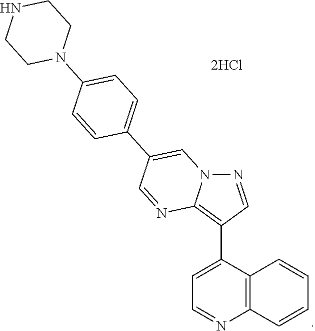 [SMILES: c1ccc2c(-c3cnn4cc(-c5ccc(N6CCNCC6)cc5)cnc34)ccnc2c1]

In some embodiments, the concentration of the BMP signaling pathway inhibitor can be from about 0.1-110 nM, 0.1-100 nM, or 0.1-50 nM. In some embodiments, the concentration of the BMP signaling pathway inhibitor can be about 100 nM. In some embodiments, the protein BMP signaling pathway inhibitor is LDN193189 and the concentration of the activator is about 100 nM.

### Rho-Associated Protein Kinase (ROCK) Inhibitor

Exemplary ROCK inhibitors include, but are not limited to a small organic molecule ROCK inhibitor selected from the group consisting of N-[(15)-2-Hydroxy-1-phenylethyl]-iV-[4-(4-pyridinyl)phenyl]-urea (AS 1892802), fasudil hydrochloride (also known as HA 1077), -[3-[[2-(4-Amino-J, 2,5-oxadiazol-3-yl)-1-ethyl-1H-imidazo[4,5-c]pyridin-6-yl]oxy]phenyl]-4-[2-(4-morpholinyl)ethoxy]benzamide (GS 269962), 4˜[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (5)-(+)-2-Methyl-1-[(4-methyl-5-isoquinolinyl)sulfonyl]-hexahydro-1H-1,4-diazepine dihydrochloride (H 1152 dihydrochloride), (5)-(+)-4-Glycyl-2-methyl-1-[(4-methyl-5-isoquinolinyl)sulfonyl]-hexahydro-1H-1,4-diazepine dihydrochloride (glycyl-M 1152 dihydrochloride), N-[(3-Hydroxyphenyl)methyl]-V-[4-(4-pyridinyl)-2-thiazolyl]urea dihydrochloride (RKI 1447 dihydrochloride), (35)-1-[[2-(4-Amino-1,2,5-oxadiazol-3-yl)-1-ethyl-1H-imidazo[4,5-c]pyridin-7-yl]carbonyl]-3-pyrrolidinamine dihydrochloride (SB772077B dihydrochloride), N-[2-[2-(Dimethylamino)ethoxyJ-4-(1H-pyrazol-4-yl)phenyl-2,3-dihydro-1,4-benzodioxin-2-carboxamide dihydrochloride (SR 3677 dihydrochloride), and tra«5°-4-[(1/?)-1-Aminoethyl]-N-4-pyridinylcyclohexanecarboxamide dihydrochloride (Y-27632 dihydrochloride), N-Benzyl-[2-(pyrimidin-4-yl)amino]thiazole-4-carboxamide (Thiazovivin), Rock Inhibitor, a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody commercially available from Santa Cruz Biotechnology selected from the group consisting of Rock-1 (B 1), Rock-1 (C-19), Rock-1 (H-11), Rock-1 (G-6), Rock-1 (H-85), Rock-1 (K-18), Rock-2 (C-20), Rock-2 (D-2), Rock-2 (D-11), Rock-2 (N-19), Rock-2 (H-85), Rock-2 (30-J); a ROCK CRISPR/Cas9 knockout plasmid selected from the group consisting of Rock-1 CRISPR/Cas9 KO plasmid (h), Rock-2 CRISPR/Cas9 KO plasmid (h), Rock-1 CR1SPR/Cas9 KO plasmid (m), Rock-2 CRISPR/Cas9 KO plasmid (m); a ROCK siRNA, shRNA plasmid and/or shRNA lentiviral particle gene silencer selected from the group consisting of Rock-1 siRNA (h): sc-29473, Rock-1 siRNA (m): sc-36432, Rock-1 siRNA (r): sc-72179, Rock-2 siRNA (h): sc-29474, Rock-2 siRNA (m): sc-36433, Rock-2 siRNA (r): sc-108088.

In some embodiments, the ROCK inhibitor comprises Y-27632. In some embodiments, the ROCK inhibitor is thiazovivin.

In some embodiments, the ROCK inhibitor can have the following structure:

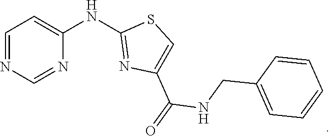 [SMILES: O=C(NCc1ccccc1)c1csc(Nc2ccncn2)n1]

In some embodiments, the concentration of the ROCK inhibitor can be from about 0.1-110 μM, 0.1-50 μM, 0.1-25 μM, or 0.1-10 μM. In some embodiments, the concentration of the ROCK inhibitor can be about 2.5 μM. In some embodiments, the ROCK inhibitor is thiazovivin and the concentration of the inhibitor is about 2.5 μM.

In some embodiments, the concentration of the ROCK inhibitor (e.g., Y-27632 or Thiazovivin), can be about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM.

### Histone Methyltransferase Inhibitors

In some embodiments, a histone methyltransferase inhibitor may be used as an epigenetic modifier. Exemplary histone methyltransferase inhibitors can include, but are not limited to, e.g., 3-Deazaneplanocin A hydrochloride (DZNep-(1S,2R,5R)-5-(4-amino-1H-imidazo[4,5-c]pyridin-1-yl)-3-(hydroxymethyl)cyclopent-3-ene-1,2-diol); Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor (serpin) family member. In some embodiments, the histone methyltransferase inhibitor can be DZNep. In some embodiments, the histone methyltransferase inhibitor can have the following structure:

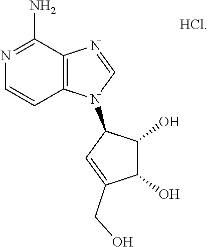 [SMILES: Nc1nccc2c1ncn2[C@@H]1C=C(CO)[C@@H](O)[C@H]1O ; "HCl."]

In some embodiments, the concentration of the histone methyltransferase inhibitor can be from about 0.1-110 nM, 0.1-100 nM, or 0.1-50 nM. In some embodiments, the concentration of the histone methyltransferase inhibitor can be about 100 nM. In some embodiments, the histone methyltransferase inhibitor is DZNep and the concentration of the inhibitor is about 100 nM.

In some embodiments, the concentration of the histone methyltransferase inhibitor can be about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM.

### MGLL Inhibitors

Exemplary MGLL (Monoglyceride Lipase) inhibitors include, but are not limited to, e.g., JJKK048, KML29, NF1819, JW642, JZL184, JZL195, JZP361, pristimerin, or URB602.

In some embodiments, the MGLL inhibitor can be JJKK048. In some embodiments, the MGLL inhibitor can be KML29. In some embodiments, the MGLL inhibitor can be NF1819.

In some embodiments, the MGLL inhibitor can have the following structure:

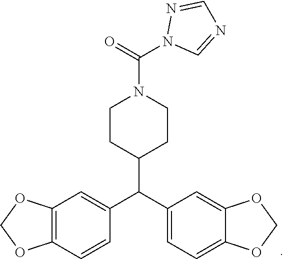 [SMILES: O=C(N1CCC(C(c2ccc3c(c2)OCO3)c2ccc3c(c2)OCO3)CC1)n1cncn1]

In some embodiments, the MGLL inhibitor can have the following structure:

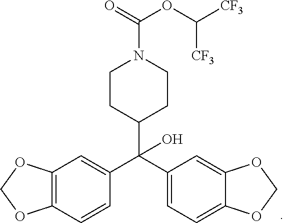 [SMILES: O=C(OC(C(F)(F)F)C(F)(F)F)N1CCC(C(O)(c2ccc3c(c2)OCO3)c2ccc3c(c2)OCO3)CC1]

In some embodiments, the MGLL inhibitor can have the following structure:

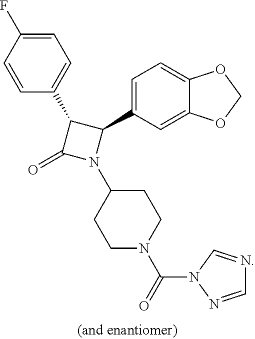 [SMILES: O=C1[C@H](c2ccc(F)cc2)[C@@H](c2ccc3c(c2)OCO3)N1C1CCN(C(=O)n2cncn2)CC1]

In some embodiments, the concentration of the MGLL inhibitor is from about 0.1 μM-100 μM. In some embodiments, the concentration of the MGLL inhibitor is about 0.1p M, 1 μM, 10 μM, 20 μM, 30 μM, 40 μM, 50 μM, 60 μM, 70 μM, 80 μM, 90 μM, 100 μM.

In some embodiments, the MGLL inhibitor is JJKK048 and the concentration is from about 0.1 μM-100 μM. In some embodiments, the MGLL inhibitor is KML29 and the concentration is from about 0.1 μM-100 μM. In some embodiments, the MGLL inhibitor is NF1819 and the concentration is from about 0.1 μM-100 μM.

In some embodiments, the MGLL inhibitor is JJKK048 and the concentration is 1 μM. In some embodiments, the MGLL inhibitor is KML29 and the concentration is 10 μM. In some embodiments, the MGLL inhibitor is NF1819 and the concentration is 10 μM.

### Lipids

Exemplary lipids include, but are not limited to, fatty acids, e.g., a saturated fatty acid or a unsaturated fatty acid. In some embodiments, the lipid is a saturated fatty acid. In some embodiments, the lipid is a unsaturated fatty acid. Exemplary saturated fatty acids include, but are not limited to, e.g., palmitate, palmitic acid, butyric acid, valeric acid, caproic acid, enanthic acid, caprylic acid, pelargonic acid, capric acid, undecylic acid, lauric acid, tridecylic acid, myristic acid, pentadecylic acid, margaric acid, Stearic acid, Nonadecylic acid, Arachidic acid, Heneicosylic acid, Behenic acid, Tricosylic acid, Lignoceric acid, Pentacosylic acid, Cerotic acid, Heptacosylic acid, Montanic acid, Nonacosylic acid, Melissic acid, Hentriacontylic acid, Lacceroic acid, Psyllic acid, Geddic acid, Ceroplastic acid, Hexatriacontylic acid, Heptatriacontanoic acid, Octatriacontanoic acid, Nonatriacontanoic acid, or Tetracontanoic acid; or a salt or ester thereof.

In some embodiments, the saturated fatty acid is palmitic acid, or a salt or ester thereof. In some embodiments, the saturated fatty acid is palmitate.

Exemplary unsaturated fatty acids include, but are not limited to, e.g., oleic acid, linoleic acid, palmitoleic acid, stearidonic acid, eicosapentaenoic acid, docosahexaenoic acid, linolelaidic acid, γ-Linolenic acid, dihomo-γ-linolenic acid, arachidonic acid, docosatetraenoic acid, vaccenic acid, paullinic acid, elaidic acid, gondoic acid, erucic acid, nervonic acid, or mead acid, or a salt or ester thereof.

In some embodiments, the unsaturated fatty acid is oleic acid. In some embodiments, the unsaturated fatty acid is linoleic acid. In some embodiments, the unsaturated fatty acid is palmitoleic acid.

### Additional Reagents

In some embodiments, any of the compositions disclosed herein comprises DMEM/F12. In some embodiments, any of the compositions disclosed herein comprises any one or more of the following amino acids: glycine (e.g., at a concentration of 0.25 mM), L-alanine (e.g., at a concentration of 0.049999997 mM), L-arginine hydrochloride (e.g., at a concentration of 0.69905216 mM), L-asparagine-H20 (0.05 mM), L-aspartic acid (e.g., at a concentration of 0.05 mM), L-Cysteine hydrochloride-H₂O (e.g., at a concentration of 0.09977272 mM), L-Cystine 2HCl (e.g., at a concentration of 0.09996805 mM), L-Glutamic Acid (e.g., at a concentration of 0.05 mM), L-glutamine (e.g., at a concentration of 2.5 mM), L-Histidine hydrochloride-H₂O (e.g., at a concentration of 0.14990476 mM), L-isoleucine (e.g., at a concentration of 0.41580153 mM), L-leucine (e.g., at a concentration of 0.45076334 mM), L-Lysine hydrochloride (e.g., at a concentration of 0.4986339 mM), L-methionine (e.g., at a concentration of 0.11570469 mM), L-phenylalanine (e.g., at a concentration of 0.2150303 mM), L-proline (e.g., at a concentration of 0.15 mM), L-serine (e.g., at a concentration of 0.25 mM), L-threonine (e.g., at a concentration of 0.44915968 mM), L-tryptophan (e.g., at a concentration of 0.04421569 mM), L-tyrosine (e.g., at a concentration of 0.21375479 mM), or L-valine (e.g., at a concentration of 0.4517094 mM). In some embodiments, any of the compositions disclosed herein comprises any of the following vitamins: biotin (e.g., at a concentration of 1.4344263E-5 mM), choline chloride (e.g., at a concentration of 0.06414285 mM), D-calcium pantothenate (e.g., at a concentration of 0.0046960167 mM), folic acid (e.g., at a concentration of 0.0060090707 mM), niacinamide (e.g., at a concentration of 0.016557377 mM), Pyridoxine hydrochloride (e.g., at a concentration of 0.009771844 mM), riboflavin (e.g., at a concentration of 5.824468E-4 mM), thiamine hydrochloride (e.g., at a concentration of 0.0064391694 mM), vitamin B12 (e.g., at a concentration of 5.0184503E-4 mM), or i-Inositol (e.g., at a concentration of 0.07 mM). In some embodiments, any of the compositions disclosed herein comprises any of the following components: dextrose (17.505556 mM), Hypoxanthine Na (0.015031448 mM), Linoleic Acid (1.4999999E-4 mM), Lipoic Acid (5.097087E-4 mM), Phenol Red (0.021519661 mM), Putrescine 2HCl (5.031056E-4 mM), Sodium Pyruvate (0.5 mM), or Thymidine (0.0015082645 mM).

In some embodiments, any of the compositions disclosed herein comprises about 0.01%, about 0.05%, about 0.1%, about 1%, about 2%, about 3%, about 4%, about 5%, about 10%, or about 15% human serum albumin (HSA). In some embodiments, the composition comprises 0.01-1%, 0.01-0.08%, 0.02-0.07%, 0.03-0.06%, 0.04-0.06%, 0.045-0.055%, 0.8-1.2%, or 0.9-1.1% HSA.

In some embodiments, any of the compositions disclosed herein comprises zinc (ZnSO₄). In some embodiments, the composition comprises 1-100 μM, 1-50 μM, 1-20 μM, 1-12 μM, 5-15 μM, 8-12 μM or 9-11 μM of ZnSO₄. In some embodiments, the composition comprises about 10 μM of ZnSO4.

In some embodiments, any of the compositions disclosed herein does not comprise ascorbic acid.

### Exemplary Agent Combinations

In some embodiments, a composition of the disclosure comprises two or more agents disclosed herein in combination. The two or more agents can be present in cell culture media, optionally media that contains cells undergoing a differentiation or reprogramming method of the disclosure. Combinations of agents disclosed herein exhibit surprising and unexpected benefits for production of SC-β cells in vitro, e.g., resulting in higher cell yields and recoveries, increased numbers and relative percentages of SC-β cells (e.g., NKX6.1-positive and/or ISL1-positive cells), enhanced stability and shelf-life of SC-β cells, SC-islet clusters with advantageous characteristics such as reduced size and increased uniformity, improved function of the SC-β cells in vitro, and improved viability, function, and reduced immunogenicity after transplantation.

In some embodiments, a combination of agents improves the fitness and metabolic flexibility of differentiating cells and the resulting SC-β cells by targeting specific aspects of cellular metabolism. Non-limiting pathways and aspects of metabolism that can be modulated by a combination of agents include one-carbon metabolism, acetyl-CoA synthesis for the generation of lipids and acetylation of proteins, fueling mitochondrial oxidative phosphorylation and TCA cycle, and generate intermediates to maintain redox homeostasis.

In some embodiments, contacting a population of cells with the combination of agents increases growth of the population of cells. In some embodiments, contacting a population of cells with the combination of agents alters or increases metabolism of the population of cells. For example, in some cases, contacting a population of cells with the combination of agents increases an oxygen consumption rate (OCR) of the population of cells by at least 5%, at least 10%, at least 15%, at least 20%, at least 25%, at least 30%, at least 35%, at least 40%, or at least 50%, e.g., as determined by a Seahorse assay. In some cases, contacting a population of cells with the combination of agents increase a population of cell's rate of ATP linked oxygen consumption by at least 5%, at least 10%, at least 15%, at least 20%, at least 25%, at least 30%, at least 35%, at least 40%, or at least 50%.

In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. A combination of agents that comprises acetate, R hydroxybutyrate, taurine, formate, biotin, and glutamine can be referred to collectively as “MQ” herein. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises at least one of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises two of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises three of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises four of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises five of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine). In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and glutamine. In some embodiments, a composition of the disclosure comprises a one carbon metabolism pathway intermediate (e.g., formate), an acetyl CoA-related metabolite (e.g., acetate), a vitamin (e.g., biotin), a redox homeostasis regulator (e.g., taurine), and glutamine. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), a redox homeostasis regulator (e.g., taurine), and glutamine. In some embodiments, a composition of the disclosure comprises a one carbon metabolism pathway intermediate (e.g., formate), a vitamin (e.g., biotin), an HDAC inhibitor (e.g., β-Hydroxybutyrate), a redox homeostasis regulator (e.g., taurine), and glutamine. In some embodiments, a composition of the disclosure comprises an acetyl CoA-related metabolite (e.g., acetate), a vitamin (e.g., biotin), an HDAC inhibitor (e.g., β-Hydroxybutyrate), a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises a one carbon metabolism pathway intermediate (e.g., formate), an acetyl CoA-related metabolite (e.g., acetate), a vitamin (e.g., biotin), and an HDAC inhibitor (e.g., β-Hydroxybutyrate). In some embodiments, a composition of the disclosure comprises a one carbon metabolism pathway intermediate (e.g., formate), an acetyl CoA-related metabolite (e.g., acetate), a vitamin (e.g., biotin), and a redox homeostasis regulator (e.g., taurine). In some embodiments, a composition of the disclosure comprises a one carbon metabolism pathway intermediate (e.g., formate), an acetyl CoA-related metabolite (e.g., acetate), a vitamin (e.g., biotin), and glutamine. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), a redox homeostasis regulator (e.g., taurine). In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), and glutamine. In some embodiments, a composition of the disclosure comprises a one carbon metabolism pathway intermediate (e.g., formate), an acetyl CoA-related metabolite (e.g., acetate), a redox homeostasis regulator (e.g., taurine), and glutamine. In some embodiments, a composition of the disclosure comprises a one carbon metabolism pathway intermediate (e.g., formate), a vitamin (e.g., biotin), an HDAC inhibitor (e.g., β-Hydroxybutyrate), and a redox homeostasis regulator (e.g., taurine). In some embodiments, a composition of the disclosure comprises a one carbon metabolism pathway intermediate (e.g., formate), a vitamin (e.g., biotin), an HDAC inhibitor (e.g., β-Hydroxybutyrate), and glutamine.

In some embodiments, a composition of the disclosure comprises a one carbon metabolism pathway intermediate (e.g., formate), a vitamin (e.g., biotin), a redox homeostasis regulator (e.g., taurine), and glutamine. In some embodiments, a composition of the disclosure comprises a one carbon metabolism pathway intermediate (e.g., formate), an HDAC inhibitor (e.g., β-Hydroxybutyrate), a redox homeostasis regulator (e.g., taurine), and glutamine. In some embodiments, a composition of the disclosure comprises an acetyl CoA-related metabolite (e.g., acetate), a vitamin (e.g., biotin), an HDAC inhibitor (e.g., β-Hydroxybutyrate), and a redox homeostasis regulator (e.g., taurine). In some embodiments, a composition of the disclosure comprises an acetyl CoA-related metabolite (e.g., acetate), a vitamin (e.g., biotin), an HDAC inhibitor (e.g., β-Hydroxybutyrate), and glutamine. In some embodiments, a composition of the disclosure comprises an acetyl CoA-related metabolite (e.g., acetate), a vitamin (e.g., biotin), a redox homeostasis regulator (e.g., taurine), and glutamine. In some embodiments, a composition of the disclosure comprises an acetyl CoA-related metabolite (e.g., acetate), an HDAC inhibitor (e.g., β-Hydroxybutyrate), a redox homeostasis regulator (e.g., taurine), and glutamine. In some embodiments, a composition of the disclosure comprises a vitamin (e.g., biotin), an HDAC inhibitor (e.g., β-Hydroxybutyrate), a redox homeostasis regulator (e.g., taurine), and glutamine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises at least one of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises two of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises three of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises four of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises five of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises six of: a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), and glutamate. In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., (3-Hydroxybutyrate), a redox homeostasis regulator (e.g., taurine), and carnitine. In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), glutamate, and carnitine. In some embodiments, a composition of the disclosure comprises a one carbon metabolism pathway intermediate (e.g., formate), an acetyl CoA-related metabolite (e.g., acetate), a vitamin (e.g., biotin), a redox homeostasis regulator (e.g., taurine), glutamate, and carnitine. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), a redox homeostasis regulator (e.g., taurine), glutamate, and carnitine. In some embodiments, a composition of the disclosure comprises a one carbon metabolism pathway intermediate (e.g., formate), a vitamin (e.g., biotin), an HDAC inhibitor (e.g., β-Hydroxybutyrate), a redox homeostasis regulator (e.g., taurine), glutamate, and carnitine. In some embodiments, a composition of the disclosure comprises an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and a redox homeostasis regulator (e.g., taurine). In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and glutamate. In some embodiments, a composition of the disclosure comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), and carnitine. In some embodiments, a composition of the disclosure comprises a one carbon metabolism pathway intermediate (e.g., formate), an acetyl CoA-related metabolite (e.g., acetate), a vitamin (e.g., biotin), a redox homeostasis regulator (e.g., taurine), and glutamate. In some embodiments, a composition of the disclosure comprises a one carbon metabolism pathway intermediate (e.g., formate), an acetyl CoA-related metabolite (e.g., acetate), a vitamin (e.g., biotin), a redox homeostasis regulator (e.g., taurine), and carnitine. In some embodiments, a composition of the disclosure comprises a one carbon metabolism pathway intermediate (e.g., formate), an acetyl CoA-related metabolite (e.g., acetate), a vitamin (e.g., biotin), glutamate, and carnitine. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), a redox homeostasis regulator (e.g., taurine), and glutamate. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), a redox homeostasis regulator (e.g., taurine), and carnitine. In some embodiments, a composition of the disclosure comprises a one carbon metabolism pathway intermediate (e.g., formate), an acetyl CoA-related metabolite (e.g., acetate), an HDAC inhibitor (e.g., β-Hydroxybutyrate), glutamate, and carnitine. In some embodiments, a composition of the disclosure comprises a one carbon metabolism pathway intermediate (e.g., formate), an acetyl CoA-related metabolite (e.g., acetate), a redox homeostasis regulator (e.g., taurine), glutamate, and carnitine. In some embodiments, a composition of the disclosure comprises a one carbon metabolism pathway intermediate (e.g., formate), a vitamin (e.g., biotin), an HDAC inhibitor (e.g., β-Hydroxybutyrate), a redox homeostasis regulator (e.g., taurine), and glutamate. In some embodiments, a composition of the disclosure comprises a one carbon metabolism pathway intermediate (e.g., formate), a vitamin (e.g., biotin), an HDAC inhibitor (e.g., β-Hydroxybutyrate), a redox homeostasis regulator (e.g., taurine), and carnitine. In some embodiments, a composition of the disclosure comprises a one carbon metabolism pathway intermediate (e.g., formate), a vitamin (e.g., biotin), an HDAC inhibitor (e.g., β-Hydroxybutyrate), glutamate, and carnitine. In some embodiments, a composition of the disclosure comprises a one carbon metabolism pathway intermediate (e.g., formate), a vitamin (e.g., biotin), a redox homeostasis regulator (e.g., taurine), glutamate, and carnitine. In some embodiments, a composition of the disclosure comprises a one carbon metabolism pathway intermediate (e.g., formate), an HDAC inhibitor (e.g., β-Hydroxybutyrate), a redox homeostasis regulator (e.g., taurine), glutamate, and carnitine. In some embodiments, a composition of the disclosure comprises an acetyl CoA-related metabolite (e.g., acetate), a vitamin (e.g., biotin), an HDAC inhibitor (e.g., β-Hydroxybutyrate), a redox homeostasis regulator (e.g., taurine), and glutamate. In some embodiments, a composition of the disclosure comprises an acetyl CoA-related metabolite (e.g., acetate), a vitamin (e.g., biotin), an HDAC inhibitor (e.g., β-Hydroxybutyrate), a redox homeostasis regulator (e.g., taurine), and carnitine. In some embodiments, a composition of the disclosure comprises an acetyl CoA-related metabolite (e.g., acetate), a vitamin (e.g., biotin), an HDAC inhibitor (e.g., β-Hydroxybutyrate), glutamate, and carnitine. In some embodiments, a composition of the disclosure comprises an acetyl CoA-related metabolite (e.g., acetate), a vitamin (e.g., biotin), a redox homeostasis regulator (e.g., taurine), glutamate, and carnitine. In some embodiments, a composition of the disclosure comprises an acetyl CoA-related metabolite (e.g., acetate), an HDAC inhibitor (e.g., β-Hydroxybutyrate), a redox homeostasis regulator (e.g., taurine), glutamate, and carnitine. In some embodiments, a composition of the disclosure comprises a vitamin (e.g., biotin), an HDAC inhibitor (e.g., β-Hydroxybutyrate), a redox homeostasis regulator (e.g., taurine), glutamate, and carnitine. In some embodiments, the composition comprises DMEM/F12. In some embodiments, the composition comprises zinc (e.g., ZnSO₄). In some embodiments, the composition comprises human serum albumin. The composition can comprise a plurality of PDX1-positive, NKX6.1-positive, insulin-negative cells, e.g., in a cell culture medium disclosed herein. The composition can comprise a plurality of insulin-positive endocrine progenitor cells e.g., in a cell culture medium disclosed herein. In some embodiments, the composition comprises cell clusters. In some embodiments, the cells of the culture are or are predominantly dissociated cells. In some embodiments, the cells are frozen. In some embodiments, the cells of the composition have been previously frozen.

A composition comprising the combination of agents can further comprise one or more of any of the additional factors disclosed herein, for example, a BMP signaling pathway inhibitor, LDN193189, a ROCK inhibitor, thiazovivin, Y-27632, Fasudil/HA1077, or 14-1152, a histone methyltransferase inhibitor, 3-Deazaneplanocin A hydrochloride, a TGF-β pathway inhibitor, ALK5 inhibitor II, a thyroid hormone signaling pathway activator, GC-1, T3, a protein kinase inhibitor, staurosporine, a Sonic Hedgehog pathway inhibitor, Sant1, Sant2, Sant 4, Sant4, Cur61414, forskolin, tomatidine, AY9944, triparanol, cyclopamine, a growth factor from epidermal growth factor (EGF) family, a gamma secretase inhibitor, XXI, DAPT, zinc, ZnSO4, a serum albumin protein, or derivatives thereof.

In some embodiments, the disclosure provides for a composition comprising a cryopreservative (e.g., DMSO), any of the cells disclosed herein, and any combination of any of the media ingredients (e.g., acetate, p-hydroxybutyrate, taurine, formate, biotin, L-glutamine, L-carnitine, or L-glutamate) disclosed herein. In some embodiments, at least 20%, 30%, 40%, 50%, 60%, 70%, 80%, 90%, 91%, 92%, 93%, 94%, 95%, 96%, 97%, 98%, 99%, or 100% of the cells in the composition are dissociated (not in clusters). In some embodiments, the composition is frozen. In some embodiments, the composition was previously frozen.

### Reprogramming

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the term “reprogramming factor” is intended to refer to a molecule that is associated with cell “reprogramming”, that is, differentiation, and/or de-differentiation, and/or transdifferentiation, such that a cell converts to a different cell type or phenotype. Reprogramming factors generally affect expression of genes associated with cell differentiation, de-differentiation and/or transdifferentiation. Transcription factors are examples of reprogramming factors.

The term “differentiation” and their grammatical equivalents as used herein refers to the process by which a less specialized cell (i.e., a more naive cell with a higher cell potency) becomes a more specialized cell type (i.e., a less naive cell with a lower cell potency); and that the term “de-differentiation” refers to the process by which a more specialized cell becomes a less specialized cell type (i.e., a more naive cell with a higher cell potency); and that the term “transdifferentiation” refers to the process by which a cell of a particular cell type converts to another cell type without significantly changing its “cell potency” or “naivety” level. Without wishing to be bound by theory, it is thought that cells “transdifferentiate” when they convert from one lineage-committed cell type or terminally differentiated cell type to another lineage-committed cell type or terminally differentiated cell type, without significantly changing their “cell potency” or “naivety” level.

As used herein, the term “cell potency” is to be understood as referring to the ability of a cell to differentiate into cells of different lineages. For example, a pluripotent cell (e.g., a stem cell) has the potential to differentiate into cells of any of the three germ layers, that is, endoderm (interior stomach lining, gastrointestinal tract, the lungs), mesoderm (muscle, bone, blood, urogenital), or ectoderm (epidermal tissues and nervous system), and accordingly has high cell potency; a multipotent cell (e.g., a stem cell or an induced stem cell of a certain type) has the ability to give rise to cells from a multiple, but limited, number of lineages (such as hematopoietic stem cells, cardiac stem cells, or neural stem cells, etc) comparatively has a lower cell potency than pluripotent cells. Cells that are committed to a particular lineage or are terminally differentiated can have yet a lower cell potency. Specific examples of transdifferentiation known in the art include the conversion of e.g., fibroblasts beta cells or from pancreatic exocrine cells to beta cells etc.

Accordingly, the cell may be caused to differentiate into a more naive cell (e.g., a terminally differentiated cell may be differentiated to be multipotent or pluripotent); or the cell may be caused to de-differentiate into a less naive cell (e.g., a multipotent or pluripotent cell can be differentiated into a lineage-committed cell or a terminally differentiated cell). However, in an embodiment, the cell may be caused to convert or transdifferentiate from one cell type (or phenotype) to another cell type (or phenotype), for example, with a similar cell potency level. Accordingly, in an embodiment of the present disclosure, the inducing steps of the present disclosure can reprogram the cells of the present disclosure to differentiate, de-differentiate and/or transdifferentiate. In an embodiment of the present disclosure, the inducing steps of the present disclosure may reprogram the cells to transdifferentiate.

Methods of reprogramming or inducing a particular type of cell to become another type of cell, for example, by differentiation, de-differentiation and/or transdifferentiation using one or more exogenous polynucleotide or polypeptide reprogramming factors are known to the person skilled in the art. Such methods may rely on the introduction of genetic material encoding one or more transcription factor(s) or other polypeptide(s) associated with cell reprogramming. For example, Pdx1, Ngn3 and MafA, or functional fragments thereof are all known to encode peptides that can induce cell differentiation, de-differentiation and/or transdifferentiation of the cells of the present disclosure. In some methods known to the person skilled in the art, exogenous polypeptides (e.g. recombinant polypeptides) encoded by reprogramming genes (such as the above genes) are contacted with the cells to induce, for example, cells of the present disclosure. The person skilled in the art will appreciate that other genes may be associated with reprogramming of cells, and exogenous molecules encoding such genes (or functional fragments thereof) and the encoded polypeptides are also considered to be polynucleotide or polypeptide reprogramming factors (e.g. polynucleotides or polypeptides that in turn affect expression levels of another gene associated with cell reprogramming). For example, it has been shown that the introduction of exogenous polynucleotide or polypeptide epigenetic gene silencers that decrease p53 inactivation increase the efficiency of inducing induced pluripotent stem cells (iPSC). Accordingly, exogenous polynucleotides or polypeptides encoding epigenetic silencers and other genes or proteins that may be directly or indirectly involved in cell reprogramming or increasing cell programming efficiency would be considered to constitute an exogenous polynucleotide or polypeptide reprogramming factor. The person skilled in the art will appreciate that other methods of influencing cell reprogramming exist, such as introducing RNAi molecules (or genetic material encoding RNAi molecules) that can knock down expression of genes involved in inhibiting cell reprogramming. Accordingly, any exogenous polynucleotide molecule or polypeptide molecule that is associated with cell reprogramming, or enhances cell reprogramming, is to be understood to be an exogenous polynucleotide or polypeptide reprogramming factor as described herein.

In some embodiments of the present disclosure, the method excludes the use of reprogramming factor(s) that are not small molecules. However, it will be appreciated that the method may utilize tissue culture components such as culture media, serum, serum substitutes, supplements, antibiotics, etc, such as RPMI, Renal Epithelial Basal Medium (REBM), Dulbecco's Modified Eagle Medium (DMEM), MCDB131 medium, CMRL 1066 medium, F12, fetal calf serum (FCS), foetal bovine serum (FBS), bovine serum albumin (BSA), D-glucose, L-glutamine, GlutaMAX™-1 (dipeptide, L-alanine-L-glutamine), B27, heparin, progesterone, putrescine, laminin, nicotinamide, insulin, transferrin, sodium selenite, selenium, ethanolamine, human epidermal growth factor (hEGF), basic fibroblast growth factor (bFGF), hydrocortisone, epinephrine, normacin, penicillin, streptomycin, gentamicin and amphotericin, etc. It is to be understood that these tissue culture components (and other similar tissue culture components that are routinely used in tissue culture) are not small molecule reprogramming molecules for the purposes of the present disclosure. Indeed, these components are either not small molecules as defined herein and/or are not reprogramming factors as defined herein. Cell culture components and metabolites disclosed herein can be used, however, to enhance the cell reprogramming and differentiation methods disclosed herein. For example, combinations of cell culture components/additives and metabolites disclosed herein can improve the efficiency of generation of SC-β cells, and their functions.

Accordingly, in an embodiment, the present disclosure does not involve a culturing step of the cell(s) with one or more exogenous polynucleotide or polypeptide reprogramming factor(s). Accordingly, in an embodiment, the method of the present disclosure does not involve the introduction of one or more exogenous polynucleotide or polypeptide reprogramming factor(s), e.g., by introducing transposons, viral transgenic vectors (such as retroviral vectors), plasmids, mRNA, miRNA, peptides, or fragments of any of these molecules, that are involved in producing induced beta cells or, otherwise, inducing cells of the present disclosure to differentiate, de-differentiation and/or transdifferentiate.

That is, in an embodiment, the method occurs in the absence of one or more exogenous polynucleotide or polypeptide reprogramming factor(s). Accordingly, it is to be understood that in an embodiment, the method of the present disclosure utilizes small molecules to reprogram cells, without the addition of polypeptide transcription factors; other polypeptide factors specifically associated with inducing differentiation, de-differentiation, and/or transdifferentiation; polynucleotide sequences encoding polypeptide transcription factors, polynucleotide sequences encoding other polypeptide factors specifically associated with inducing differentiation, de-differentiation, and/or transdifferentiation; mRNA; interference RNA; microRNA and fragments thereof.

### Stem Cells

The term “stem cell” is used herein to refer to a cell (e.g., plant stem cell, vertebrate stem cell) that has the ability both to self-renew and to generate a differentiated cell type (Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., beta cell progenitors), which can differentiate into end-stage cells (i.e., terminally differentiated cells, e.g., beta cells, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

By “embryonic stem cell” (ESC) is meant a PSC that is isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, butnot SSEA-1. Examples of methods of generating and characterizing ESCs may be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety.

By “embryonic germ stem cell” (EGSC) or “embryonic germ cell” or “EG cell”, it is meant a PSC that is derived from germ cells and/or germ cell progenitors, e.g. primordial germ cells, i.e. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

By “induced pluripotent stem cell” or “iPSC”, it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell”, it is meant any cell in an organism that, in the absence of experimental manipulation, does not ordinarily give rise to all types of cells in an organism. In other words, somatic cells are cells that have differentiated sufficiently that they do not naturally generate cells of all three germ layers of the body, i.e. ectoderm, mesoderm and endoderm. For example, somatic cells can include both neurons and neural progenitors, the latter of which is able to naturally give rise to all or some cell types of the central nervous system but cannot give rise to cells of the mesoderm or endoderm lineages

In certain examples, the stem cells can be undifferentiated (e.g. a cell not committed to a specific lineage) prior to exposure to at least one β cell maturation factor according to the methods as disclosed herein, whereas in other examples it may be desirable to differentiate the stem cells to one or more intermediate cell types prior to exposure of the at least one cell maturation factor (s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be by themselves (for example, without substantially any undifferentiated cells being present) or may be used in the presence of differentiated cells. In certain examples, the stem cells may be cultured in the presence of) suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, FISF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al, (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al, (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. In an embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the SC-β cell can be derived from one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic F cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

### Pancreatic Progenitor Cells or Precursors

In some aspects, the present disclosure provides a method of producing a NKX6.1-positive pancreatic progenitor cell from a Pdx1-positive pancreatic progenitor cell comprising contacting a population of cells comprising Pdx1-positive pancreatic progenitor cells or precursors under conditions that promote cell clustering with at least two β cell-maturation factors comprising a) at least one growth factor from the fibroblast growth factor (FGF) family, b) a sonic hedgehog pathway inhibitor, and optionally c) a low concentration of a retinoic acid (RA) signaling pathway activator, for a period of at least five days to induce the differentiation of at least one Pdx1-positive pancreatic progenitor cell in the population into NKX6.1-positive pancreatic progenitor cells, wherein the NKX6.1-positive pancreatic progenitor cells express NKX6.1.

In some embodiments, at least 10% of the Pdx1-positive pancreatic progenitor cells in the population are induced to differentiate into NKX6-1-positive pancreatic progenitor cells. In some embodiments, at least 95% of the Pdx1-positive pancreatic progenitor cells in the population are induced to differentiate into NKX6.1-positive pancreatic progenitor cells. In some embodiments, the NKX6.1-positive pancreatic progenitor cells express Pdx1, NKX6.1, and FoxA2. In some embodiments, the Pdx1-positive pancreatic progenitor cells are produced from a population of pluripotent stem cells selected from the group consisting of embryonic stem cells and induced pluripotent stem cells.

### Stem Cell Derived Beta Cells

In some embodiments, provided herein are methods of using of stem cells to produce SC-beta cells (e.g., mature pancreatic β cells or β-like cells) or precursors thereof. In an embodiment, germ cells may be used in place of, or with, the stem cells to provide at least one SC-β cell, using similar protocols as described in U.S. Patent Application Publication No. US20150240212 and US20150218522, each of which is herein incorporated by reference in its entirety. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

In some embodiments, provided herein are compositions and methods of generating SC-β cells (e.g., pancreatic β cells). Generally, the at least one SC-β cell or precursor thereof, e.g., pancreatic progenitors produced according to the methods disclosed herein can comprise a mixture or combination of different cells, e.g., for example a mixture of cells such as a Pdx1-positive pancreatic progenitors, pancreatic progenitors co-expressing Pdx1 and NKX6.1, a Ngn3-positive endocrine progenitor cell, an insulin-positive endocrine cell (e.g., a β-like cell), and an insulin-positive endocrine cell, and/or other pluripotent or stem cells.

The at least one SC-β cell or precursor thereof can be produced according to any suitable culturing protocol to differentiate a stem cell or pluripotent cell to a desired stage of differentiation. In some embodiments, the at least one SC-β cell or the precursor thereof are produced by culturing at least one pluripotent cell for a period of time and under conditions suitable for the at least one pluripotent cell to differentiate into the at least one SC-β cell or the precursor thereof. In some embodiments, production of the SC-β cell or precursor thereof is enhanced by contacting the cell with one or more agents disclosed herein (e.g., one or more of glutamine, glutamate, carnitine, taurine, β-hydroxybutyrate, biotin, acetate, and formate).

In some embodiments, the at least one SC-β cell or precursor thereof is a substantially pure population of SC-β cells or precursors thereof. In some embodiments, a population of SC-β cells or precursors thereof comprises a mixture of pluripotent cells or differentiated cells. In some embodiments, a population SC-β cells or precursors thereof are substantially free or devoid of embryonic stem cells or pluripotent cells or iPS cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce the at least one SC-β cell or precursor thereof for use in the compositions and methods described herein. In some embodiments, a somatic cell, e.g., fibroblast is maintained in culture by methods known by one of ordinary skill in the art, and in some embodiments, propagated prior to being converted into SC-β cells by the methods as disclosed herein.

In some embodiments, the at least one SC-β cell or precursor thereof are maintained in culture by methods known by one of ordinary skill in the art, and in some embodiments, propagated prior to being converted into SC-β cells by the methods as disclosed herein.

Further, at least one SC-β cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian at least one SC-β cell or precursor thereof but it should be understood that all of the methods described herein can be readily applied to other cell types of at least one SC-β cell or precursor thereof. In some embodiments, the at least one SC-β cell or precursor thereof is derived from a human individual.

In some embodiments, a population of cells of the disclosure comprises a plurality of cells expressing both NKX6.1 and ISL1. For example, at least about 10%, 20%, 30%, 35%, 38%, 40%, 42%, 44%, 46%, 50%, 60%, 70%, 80%, 90%, 95%, or 99% cells can express ISL1. In some cases, at least about 35% of cells express NKX6.1 and ISL1. In some cases, at least about 38% of cells express NKX6.1 and ISL1. In some cases, at least about 40% of cells express NKX6.1 and ISL1. In some cases, at least about 42% of cells express NKX6.1 and ISL1. In some cases, at least about 44% of cells express NKX6.1 and ISL1. In some cases, at least about 46% of cells express NKX6.1 and ISL1. In some cases, at least about 50% of cells express NKX6.1 and ISL1. Compositions and methods of the disclosure can be useful for increasing the percentage of cells that express both NKX6.1 and ISL1. For example, after contacting cells undergoing differentiation into SC-β cells according to methods of the disclosure, a percentage of cells that express NKX6.1 and ISL1 can be increased relative to methods that lack one or more of the agents, or that contain different (e.g., lower) concentrations of them.

In some cases, a population of cells of the disclosure contains a limited proportion of cells that are negative for both NKX6.1 and ISL1. For example, a cell population can comprise at most 2%, at most 4%, at most 6%, at most 8%, at most 10%, at most 12%, at most 14%, at most 16%, at most 18%, at most 20%, at most 22%, at most 22%, at most 25%, or at most 30% ISL1-negative, NKX6.1-negative cells.

### Cell Clusters of Stem Cell Derived Beta Cells

In some aspects, provided herein are cell clusters that resemble the functions and characteristics of endogenous pancreatic islets. Such cell clusters can mimic the function of endogenous pancreatic islets in regulating metabolism, e.g., glucose metabolism in a subject. Thus, the cell clusters can be transplanted to a subject for treating disease resulting from insufficient pancreatic islet function, e.g., diabetes. The terms “cluster” and “aggregate” can be used interchangeably, and refer to a group of cells that have close cell-to-cell contact, and in some cases, the cells in a cluster can be adhered to one another.

A cell cluster comprises a plurality of cells. In some embodiments, a cell cluster comprises at least 10, at least 50, at least 200, at least 500, at least 750, at least 1000, at least 1500, at least 2000, at least 2500, at least 3000, at least 3500, at least 4000, at least 4500, at least 5000, at least 6000, at least 7000, at least 8000, at least 9000, at least 10,000, at least 20,000, at least 30,000, or at least 50,000 cells. In some embodiments, a cell cluster comprises between 10-10,000 cells, between 50-10,000, between 100-10,000, between 100-10,000, between 1,000-10,000, between 500 and 10,000, between 500 and 5,000, between 500 and 2,500, between 500 and 2,000, between 1,000 and 100,000, between 1,000 and 50,000, between 1,000 and 40,000, between 1,000 and 20,000, between 1,000 and 10,000, between 1,000 and 5,000 and between 1,000 and 3,000 cells. In some embodiments, a cell cluster comprises at least 500 cells. In some embodiments, a cell cluster comprises at least 1,000 cells. In some embodiments, a cell cluster comprises at least 2,000 cells. In some embodiments, a cell cluster comprises at least 5,000 cells. In some embodiments, a cell cluster comprises no more than 100,000, no more than 90,000, no more than 80,000, no more than 70,000, no more than 60,000, no more than 50,000, no more than 40,000, no more than 30,000, no more than 20,000, no more than 10,000, no more than 7,000, no more than 5,000, no more than 3,000, no more than 2,000 cells, or no more than 1,000 cells.

A cell cluster herein can comprise at least one non-native cell, e.g., a non-native pancreatic β cell. A non-native cell (e.g., a non-native pancreatic β cell) can share characteristics of an endogenous cell (e.g., an endogenous mature pancreatic β cell), but is different in certain aspects (e.g., gene expression profiles). A non-native cell can be a genetically modified cell. A non-native cell can be a cell differentiated from a progenitor cell, e.g., a stem cell. The stem cell can be an embryonic stem cell (ESC) or induced pluripotent stem cell (iPSC). In some cases, the non-native cell can be a cell differentiated from a progenitor cell in vitro. In some cases, the non-native cell can be a cell differentiated from a progenitor cell in in vivo. For example, a cell cluster can comprise at least one non-native pancreatic β cell. The non-native pancreatic β cells can be those described in U.S. patent application Ser. Nos. 14/684,129 and 14/684,101, which are incorporated herein in their entireties. A cell cluster can comprise a plurality of non-native pancreatic β cells. In some cases, at least about 20%, 30%, 40%, 50%, 60%, 70%, 80%, 90%, 99% cells in a cell cluster are non-native pancreatic β cells. A cell cluster can comprise one or more native cells. For example, a cell cluster can comprise one or more primary cells, e.g., primary cells from an endogenous pancreatic islet.

A cell cluster can comprise one or more cells expressing at least one marker of an endogenous cell, e.g., an endogenous mature pancreatic β cell. The term “marker” can refer to a molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein, or a small molecule. In many cases, a marker can refer to a molecule that can be characteristic of a particular type of cell, so that the marker can be called as a marker of the type of cell. For instance, Insulin gene can be referred to as a marker of β cells. In some cases, a marker is a gene. Non-limiting of markers of an endogenous mature pancreatic β cell include insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN.

A cell cluster can comprise one more cells expressing one or multiple markers of an endogenous cell, e.g., an endogenous mature pancreatic β cell. For example, cell cluster can comprise one or more cells co-expressing at least 2, 3, 4, 5, 6, 7, 8, 9, 10, 15, or 20 marker(s) of an endogenous cell, e.g., an endogenous mature pancreatic β cell. In some cases, a cell cluster comprises cells that express NKX6.1 and C-peptide, both of which can be markers of a β cell.

A cell cluster can comprise a plurality of cells expressing at least one marker of an endogenous cell. For example, at least about 1%, 5%, 10%, 20%, 30%, 40%, 50%, 60%, 70%, 80%, 90%, 95%, 99% cells in a cell cluster can express at least one marker of an endogenous cell. In some cases, all cells in a cell cluster can express a marker of an endogenous cell. In some cases, the endogenous cell can be a pancreatic cell, e.g., a pancreatic β cell, pancreatic α cells, pancreatic β cells, pancreatic A cells, or pancreatic γ cells. A cell cluster as provided herein can comprise a heterogeneous group of cells, e.g., cells of different types. For example, the cell cluster can comprises a cell expressing insulin/C-peptide, which can be a marker of a pancreatic β cell, a cell expressing glucagon, which can be a marker of a pancreatic α cell, a cell expressing somatostatin, which can be a marker of a pancreatic A cell, a cell expressing pancreatic polypeptides, or any combination thereof.

For example, the cell cluster herein can comprise a plurality of cells expressing one or more markers of an endogenous mature pancreatic β cell. For example, at least about 10%, 20%, 30%, 40%, 50%, 60%, 70%, 80%, 90%, 95%, or 99% cells in the cell cluster can express one or more markers of an endogenous mature pancreatic β cell.

The cell cluster can comprise a plurality of cells expressing CHGA. In some cases, at least about 30%, 40%, 50%, 60%, 70%, 80%, 90%, 95%, or 99% cells in the cell cluster express CHGA. In some cases, at least about 85% cells in a cell cluster can express CHGA. In some cases, a cell cluster can comprise about 90% cell expressing CHGA. In some cases, a cell cluster can comprise about 95% cells expressing CHGA. In certain cases, all cells in a cell cluster can express CHGA.

The cell cluster can comprise a plurality of cells expressing NKX6.1. For example, at least about 30%, 40%, 50%, 60%, 70%, 80%, 90%, 95%, 99% cells in a cell cluster can express NKX6.1. In some cases, at least about 50% cells in a cell cluster can express NKX6.1. In some cases, all cells in a cell cluster can express NKX6.1.

The cell cluster can comprise a plurality of cells expressing ISL1. For example, at least about 30%, 40%, 50%, 60%, 70%, 80%, 90%, 95%, 99% cells in a cell cluster can express NKX6.1. In some cases, at least about 50% cells in a cell cluster can express ISL1. In some cases, all cells in a cell cluster can express ISL1.

The cell cluster can comprise a plurality of cells expressing C-peptide. For example, at least about 30%, 40%, 50%, 60%, 70%, 80%, 90%, 95%, or 99% cells in a cell cluster can express C-peptide. In some cases, at least about 60% cells in a cell cluster can express C-peptide. In some cases, all cells in a cell cluster can express C-peptide.

The cell cluster can comprise a plurality of cells expressing both NKX6.1 and C-peptide. For example, at least about 10%, 20%, 30%, 40%, 50%, 60%, 70%, 80%, 90%, 95%, or 99% cells in a cell cluster can express C-peptide. In some cases, at least about 35% cells in a cell cluster can express NKX6.1 and C-peptide. In some cases, at least about 40% cells in a cell cluster can express NKX6.1 and C-peptide. In some cases, at least about 35% cells in a cell cluster can express NKX6.1 and C-peptide. In some cases, a cell cluster can comprise about 60% cells expressing NKX6.1 and C-peptide. In some cases, a cell cluster can comprise about 75% cell expressing NKX6.1 and C-peptide. In some cases, all cells in a cell cluster can express NKX6.1 and C-peptide.

The cell cluster can comprise a plurality of cells expressing both NKX6.1 and ISL1. For example, at least about 10%, 20%, 30%, 35%, 38%, 40%, 42%, 44%, 46%, 50%, 60%, 70%, 80%, 90%, 95%, or 99% cells in a cell cluster can express NKX6.1 and ISL1. In some cases, at least about 35% cells in a cell cluster can express NKX6.1 and ISL1. In some cases, at least about 40% cells in a cell cluster can express NKX6.1 and ISL1. In some cases, at least about 35% cells in a cell cluster can express NKX6.1 and ISL1. In some cases, a cell cluster can comprise about 60% cells expressing NKX6.1 and ISL1. In some cases, a cell cluster can comprise about 75% cell expressing NKX6.1 and ISL1. In some cases, all cells in a cell cluster can express NKX6.1 and ISL1.

The cell cluster can comprise a limited proportion of cells that are negative for both NKX6.1 and ISL1. For example, a cell cluster can comprise at most 2%, at most 4%, at most 6%, at most 8%, at most 10%, at most 12%, at most 14%, at most 16%, at most 18%, at most 20%, at most 22%, at most 22%, at most 25%, or at most 30% ISL1-negative, NKX6.1-negative cells.

The cell cluster can comprise very few to none of stem cells or progenitor cells, e.g., pancreatic progenitor cells. For example, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67.

In some cases, a cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, a cell cluster can comprise about 1% cells expressing SOX2. In some cases, a cell cluster can comprise about 0.6% cells expressing SOX2. In some cases, a cell cluster can comprise about 0.3% cells expressing SOX2. In some cases, a cell cluster can comprise about 0.1% cells expressing SOX2.

In some examples, a cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, a cell cluster can comprise about 2% cells expressing SOX9. In some cases, a cell cluster can comprise about 6% cells expressing SOX9. In some cases, a cell cluster can comprise about 1.2% cells expressing SOX9.

A cell cluster herein can exhibit one or multiple glucose stimulated insulin secretion (GSIS) response(s) in vitro when exposed to glucose challenge(s). The GSIS responses can resemble the GSIS responses of an endogenous pancreatic islet. In some cases, the cell cluster exhibits an in vitro GSIS response to a glucose challenge. In some cases, the cell cluster exhibits in vitro GSIS responses to multiple glucose challenges, such as sequential glucose challenges. For example, the cell cluster can exhibit in vitro GSIS responses to at least 2, 3, 4, 5, 6, 7, 8, 9, 10 sequential glucose challenges.

A cell cluster as provided herein can comprise at least one cell exhibiting in vitro GSIS. For example, at least one cell in the cell cluster can be referred to as a mature pancreatic β cell. In some cases, the at least one cell is a non-native pancreatic β cell. In some cases, the at least one cell is a pancreatic β cell resembling a native/endogenous β cell. In some cases, the cell exhibits an in vitro glucose stimulated insulin secretion (GSIS) response. In some cases, the at least one cell exhibits a GSIS response to at least one glucose challenge. In some cases, the cell exhibits a GSIS response to at least two sequential glucose challenges. In some cases, the cell exhibits a GSIS response to at least three sequential glucose challenges.

As provided herein, a cell cluster can exhibit GSIS stimulation index similar to an endogenous pancreatic islet. Stimulation index of a cell cluster or a cell can be characterized by the ratio of insulin secreted in response to high glucose concentrations compared to low glucose concentrations. For example, a stimulation index of a cell cluster or a cell as provided herein can be calculated as a ratio of insulin secreted in response to 20 mM glucose stimulation versus insulin secreted in response to 2.8 mM glucose stimulation. In some examples, the stimulation index of a cell cluster or a cell as provided herein is greater than or equal to 1, or greater than or equal to 1.1, or greater than or equal to 1.3, or greater than or equal to 2, or greater than or equal to 2.3, or greater than or equal to 2.6. In some instances, the cell cluster or the cell exhibits cytokine-induced apoptosis in response to a cytokine. In some cases, the cytokine comprises interleukin-β (IL-β), interferon-γ (INF-γ), tumor necrosis factor-α (TNF-α), or any combination thereof. In some cases, insulin secretion from the cell cluster or the cell is enhanced in response to an anti-diabetic agent. In some cases, the anti-diabetic agent comprises a secretagogue selected from the group consisting of an incretin mimetic, a sulfonylurea, a meglitinide, and combinations thereof. In some cases, the cell cluster or the cell is monohormonal. In some cases, the cell cluster or the cell exhibits a morphology that resembles the morphology of an endogenous mature pancreatic β cell. In some cases, the cell cluster or the cell exhibits encapsulated crystalline insulin granules under electron microscopy that resemble insulin granules of an endogenous mature pancreatic β cell. In some cases, the cell cluster or the cell exhibits a low rate of replication. In some cases, the cell cluster or the cell exhibits a glucose stimulated Ca²⁺ flux (GSCF) that resembles the GSCF of an endogenous mature pancreatic β cell. In some cases, the cell cluster or the cell exhibits a GSCF response to at least one glucose challenge. In some cases, the cell cluster or the cell exhibits a GSCF response to at least two glucose challenges. In some cases, the cell cluster or the cell exhibits a GSCF response to at least three glucose challenges. In some cases, the cell cluster or the cell exhibits an increased calcium flux. In some cases, the increased calcium flux comprises an increased amount of influx or a ratio of influx at low relative to high glucose concentrations.

A cell cluster as provided herein can exhibit biphasic insulin secretion in response to a high glucose concentration stimulation similar to an endogenous pancreatic islet, e.g., a human pancreatic islet. A biphasic insulin secretion can be a phenomenon characteristic of an endogenous pancreatic islet, e.g., human islet. In some embodiments, response to a high glucose concentration challenge, e.g., 10 mM, 15 mM, 20 mM, or 30 mM, a cell cluster as provided herein, e.g., a reaggregated pancreatic cell cluster, can exhibit a transient increase in insulin secretion to a peak value followed by a rapid decrease to a relatively elevated insulin secretion level, e.g., a level that is higher than an insulin secretion level in response to a lower glucose concentration, e.g., 2.8 mM glucose. Such a transient increase and decrease process can be termed as a first phase of the biphasic insulin secretion pattern. With a persistent high glucose challenge, the first phase can be thus followed by a second phase, in which the insulin secretion by the cell cluster can be maintained at the relatively elevated level. The second phase can last for an extended period, e.g., as long as the high glucose concentration challenge lasts, or relatively longer than the first phase. Such a biphasic insulin secretion pattern can be due to intrinsic cellular signaling changes that are characteristic of a mature native pancreatic β cell.

When transplanted to a subject, a cell cluster can exhibit one or more in vivo GSIS responses when exposed to glucose challenge(s). The cell cluster herein can be capable of exhibiting an in vivo GSIS response within a short period of time after transplanted to a subject. For example, the cell cluster can exhibit an in vivo GSIS within about 6, 12, or 24 hours after transplantation. In some cases, the cell cluster exhibits an in vivo GSIS within about 2 days, 4 days, 6 days, 8 days, 10 days, 12 days, 14 days, 21 days, 28 days, 35 days, or 42 days after transplantation. The amount of insulin secreted by the cell cluster can be similar or higher than an endogenous pancreatic islet. The term “about” in relation to a reference numerical value as used through the application can include a range of values plus or minus 10% from that value. For example, the amount “about 10” includes amounts from 9 to 11. For example, the term “about” in relation to a reference numerical value can also include a range of values plus or minus 10%, 9%, 8%, 7%, 6%, 5%, 4%, 3%, 2%, or 1% from that value.

The cell cluster can maintain the ability of exhibiting in vivo GSIS responses for a period of time after transplanted into a subject. For example, an in vivo GSIS response of the cell cluster can be observed up to at least 2 weeks, 3 weeks, 4 weeks, 5 weeks, 10 weeks, 15 weeks, 20 weeks, 6 months, 7 months, 8 months, 9 months, 10 months, 11 months, 1 year, 2 years, 3 years, 4 years, 5 years, 10 years, 20 years, 30 years, 40 years, 60 years, 80 years, or 100 years after transplantation of the cell cluster into a subject (e.g., a human).

The GSIS of a cell cluster can be measured by a stimulation index. A stimulation index of a cell cluster can equal to the ratio of insulin secreted in response to a high glucose concentration compared to insulin secreted in response to a low glucose concentration. A cell cluster can have a stimulation index similar to an endogenous pancreatic islet. In some cases, a cell cluster has a stimulation index of at least 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

The amount of insulin secreted by a cell cluster in response to a glucose challenge (e.g., a high concentration, such as 20 mM, of glucose) can range from about 0.1 μIU/10³ cells to about 5 μIU/10³ cells, from about 0.2 μIU/10³ cells to about 4 μIU/10³ cells, from about 0.2 μIU/10³ cells to about 3 μIU/10³ cells, or from about 0.23 μIU/10³ cells to about 2.7 μIU/10³ cells. In some cases, the amount of insulin secreted by a cell cluster in response to a glucose challenge (e.g., a high concentration, such as 20 mM, of glucose) is at least 0.05, 0.1, 0.15, 0.2, 0.21, 0.22, 0.23, 0.24, 0.25, 0.26, 0.27, 0.28, 0.29, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μIU/10³ cells.

A cell cluster can secrete both pro-insulin and insulin. For example, a cell cluster can secrete pro-insulin and insulin at a proinsulin-to-insulin ratio substantially the same as the ratio of pro-insulin to insulin secreted by an endogenous pancreatic islet. In some cases, a cell cluster secretes pro-insulin and insulin at a proinsulin-to-insulin ratio of from about 0.01 to about 0.05, from about 0.02 to about 0.04, from about 0.02 to about 0.03, or from 0.029 to about 0.031. In some cases, a cell cluster secretes pro-insulin and insulin at a proinsulin-to-insulin ratio of about 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, or 0.04.

A cell cluster can be in a size similar to an endogenous pancreatic islet. For example, a cell cluster can have a diameter similar to an endogenous pancreatic islet. A diameter of a cell cluster can refer to the largest linear distance between two points on the surface of the cell cluster. In some cases, the diameter of a cell cluster is at most 300 μm, 200 μm, 150 μm, 100 μm, 90 μm, 80 μm, 70 μm, 60 μm, 50 μm, or 40 μm. The diameter of a cell cluster can be from about 75 μm to about 250 μm. The diameter of a cell cluster can be at most 100 μm.

In some embodiments, at least about 40%, 50%, 60%, 70%, 80%, or 90% of cell clusters have a diameter of from about 50 μm to about 250 μm, from about 75 μm to about 250 μm, or from about 100 μm to about 200 μm.

In some embodiments, at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm.

In some embodiments, the cell clusters have a mean or median diameter of at most 120, at most 130, at most 140, at most 150, at most 160, or at most 170 μm.

In some embodiments, the cell clusters have a mean or median diameter of about 80-150, about 100-150, about 120-150, about 140-150, about 80-130, about 100-130, about 120-130, about 80-120, about 90-120, or about 100-120 μm.

In some embodiments, at least about 50%, at least about 60%, at least about 70%, or at least about 80% of the cell clusters have a diameter of less than 150 μm.

In some embodiments, at least about 50%, at least about 60%, or at least about 70% of the cell clusters have a diameter of less than 140 μm.

In some embodiments, at least about 50%, at least about 60%, or at least about 70% of the cell clusters have a diameter of less than 130 μm.

A cell cluster can comprise very few or no dead cells. The cell cluster can be in a size that allows effective diffusion of molecules (e.g., nutrition and gas) from surrounding environment into the core of the cell cluster. The diffused molecule can be important for the survival and function of the cells in the core. In some cases, the cell cluster can have less than about 1%, 2%, 3%, 4%, 5%, 6%, 7%, 8%, 9%, 10% of dead cells, e.g., dead cells in its core. In some cases, a cell cluster can have no dead cell. The dead cells can be apoptotic cells, narcotic cells or any combination thereof.

A cell cluster can comprise one or multiple types of cells. In some cases, a cell cluster comprises one or more types of pancreatic cells. For example, the cell cluster can comprise one or more pancreatic β cell, pancreatic α cells, pancreatic A cells, pancreatic γ cells, and any combination thereof. In some cases, the pancreatic cells can be non-native pancreatic cells, e.g., cells derived from stem cells, such as ESCs and/or iPSCs. In some cases, the cell cluster can also comprise one or more progenitor cells of mature pancreatic cells, including iPSCs, ESCs, definitive endoderm cells, primitive gut tube cells, Pdx1-positive pancreatic progenitor cells, Pdx1-positive/NKX6.1-positive pancreatic progenitor cells, Ngn3-positive endocrine progenitor cells, and any combination thereof.

A cell cluster can exhibit cytokine-induced apoptosis in response to cytokines. For example, the cell cluster can exhibit cytokine-induced apoptosis in response to a cytokine such as interleukin-1β (IL-β), interferon-γ (INF-γ), tumor necrosis factor-α (TNF-α), and combinations thereof.

Insulin secretion from a cell cluster herein can be enhanced by an anti-diabetic drug (e.g., an anti-diabetic drug acting on pancreatic β cells ex vivo, in vitro, and/or in vivo). The disclosure can contemplate any known anti-diabetic drug. In some cases, insulin secretion from a cell cluster can be enhanced by a secretagogue. The secretagogue can be an incretin mimetic, a sulfonylurea, a meglitinide, and combinations thereof.

A cell cluster can comprise a monohormonal. For example, the cell cluster can comprise a pancreatic cell (e.g., a pancreatic β cell, pancreatic α cells, pancreatic β cells, pancreatic A cells, or pancreatic γ cells) that is monohormonal. In some cases, the cell cluster comprises an insulin-secreting non-native pancreatic cell that is monohormonal. A cell cluster can comprise a polyhormonal. In some case, a cell cluster comprises a monohormonal cell and a polyhormonal cell.

A cell cluster can comprise a cell (e.g., a non-native pancreatic cell) having a morphology that resembles the morphology of an endogenous mature pancreatic β cell. In some cases, the cell cluster can comprise cell encapsulating crystalline insulin granules that resemble insulin granules of an endogenous mature pancreatic β cell, e.g., as detected by electron microscopy. A cell cluster can comprise a plurality cells having a morphology that resembles the morphology of an endogenous mature pancreatic β cell. For example, at least about 10%, 20%, 30%, 40%, 50%, 60%, 70%, 80%, 90% cells in a cell cluster can encapsulate crystalline insulin granules that resemble insulin granules of an endogenous mature pancreatic β cell. In some cases, 100% cells in a cell cluster encapsulate crystalline insulin granules that resemble insulin granules of an endogenous mature pancreatic β cell.

A cell cluster can exhibit glucose-stimulated calcium (Ca²⁺) flux to one or more glucose challenges. In some cases, a cell cluster exhibits a glucose-stimulated Ca²⁺ flux (GSCF) that resembles the GSCF of an endogenous pancreatic islet. In some cases, a cell cluster exhibits a GSCF response to at least 1, 2, 3, 4, 5, 6, 8, or 10 sequential glucose challenges in a manner that resembles the GSCF response of an endogenous pancreatic islet to multiple glucose challenges. A cell cluster can exhibit an in vitro and/or in vivo GSCF response when exposed to a glucose challenge.

A cell cluster can comprise cells originated from any species. For example, a cell cluster can comprise cells from a mammalian species, with non-limiting examples including a murine, bovine, simian, porcine, equine, ovine, or human cell. In some cases, at least one cell in the cell cluster is a human cell.

Provided herein also include compositions comprising a cell clusters disclosed through the application. In addition to the cell cluster, the compositions can further comprise a scaffold or matrix that can be used for transplanting the cell clusters to a subject. A scaffold can provide a structure for the cell cluster to adhere to. The cell cluster can be transplanted to a subject with the scaffold. The scaffold can be biodegradable. In some cases, a scaffold comprises a biodegradable polymer. The biodegradable polymer can be a synthetic polymer, such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, and biodegradable polyurethanes. The biodegradable polymer can also be a natural polymer, such as albumin, collagen, fibrin, polyamino acids, prolamines, and polysaccharides (e.g., alginate, heparin, and other naturally occurring biodegradable polymers of sugar units). Alternatively, the scaffold can be non-biodegradable. For example, a scaffold can comprise a non-biodegradable polymer, such as polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, and polyethylene oxide.

### Dissociated Cell Compostions and Methods for Making Cell Clusters of Stem Cell Derived Beta Cells

Further disclosed herein are methods for making cell clusters that resemble the function and characteristics of an endogenous tissue or cell cluster, e.g., an endogenous pancreatic islet.

The methods can comprise dissociating a first cell cluster and re-aggregating the dissociated cells to a second cell cluster, where the second cell cluster more closely resembles the function and characteristics of an endogenous tissue or cell cluster, e.g., an endogenous pancreatic islet, compared to the first cell cluster. The term “re-aggregating” and its grammatical equivalences as used herein can refer to, when clusters are dissociated into smaller clusters or single cells, the dissociated cells then form new cell-to-cell contacts and form new clusters. The methods can be used for producing a cell cluster in vitro by a) dissociating a plurality of cells from a first cell cluster; and b) culturing the plurality of cells from a) in a medium, thereby allowing the plurality of cells to form a second cell cluster. In some cases, the second cell cluster is an in vitro cell cluster. The first cell cluster can be an in vitro cell cluster, e.g., a cluster formed by a suspension of single cells in vitro in a culture medium. In some cases, the first cell cluster can be an ex vivo cell cluster, e.g., a cell cluster that is formed in a body of a live organism and isolated from said organism. For example, a first cell cluster that the method provided herein is applicable to can be a human pancreatic islet. In some cases, the first cell cluster can be a cadaveric pancreatic islet. In some embodiments, the dissociated cells were previously frozen.

A method provided herein can enrich pancreatic cells in a cell cluster, e.g., a pancreatic β cell, an endocrine cell, or an endocrine progenitor cell. In some examples, the method can reduce or eliminate stem cells or pancreatic progenitor cells from a cell cluster. In some cases, the second cell cluster comprises a higher percentage of cells that express chromogranin A as compared the first cell cluster. In some cases, the second cell cluster comprises a higher percentage of cells that express NKX6.1 and C-peptide as compared the first cell cluster. In some cases, the second cell cluster comprises a higher percentage of cells that express NKX6.1 and ISL1 as compared the first cell cluster. In some cases, the second cell cluster comprises a lower percentage of cells that are negative for NKX6.1 and C-peptide as compared the first cell cluster. In some cases, the second cell cluster comprises a lower percentage of cells that express SOX2 as compared the first cell cluster. In some cases, the second in vitro cell cluster comprises a lower percentage of cells that express SOX9 as compared the first cell cluster.

In some cases, the medium comprises a thyroid hormone signaling pathway activator and a transforming growth factor β (TGF-0) signaling pathway inhibitor. In some cases, the medium comprises a) serum, and b) one or both of a thyroid hormone signaling pathway activator and a TGF-β signaling pathway inhibitor. In some cases, the medium for reaggregation as provided herein (reaggregation medium) can comprise no small molecule compounds. For example, the reaggregation medium can comprise no thyroid hormone signaling pathway activator. In some cases, the reaggregation medium does not comprise triiodothyronine (T3), or merely a trace amount of T3. The reaggregation medium can comprise no TGFβ signaling pathway inhibitor. In some cases, the reaggregation medium does not comprise an Alk5 inhibitor (Alk5i), or merely a trace amount of Alk5i.

In some cases, reaggregation medium comprises one or more of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine.

In some cases, reaggregation medium comprises 2, 3, 4, 5, or 6 of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine.

In some cases, reaggregation medium comprises a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamate, and carnitine.

In some embodiments, the method comprises contacting the population of cells (e.g., NKX6.1-positive, ISL1-positive, insulin-positive cells) with one or more of a serum albumin protein, a TGF-β signaling pathway inhibitor, a TH signaling pathway activator, a protein kinase inhibitor, a ROCK inhibitor, a BMP signaling pathway inhibitor, an epigenetic modifying compound, acetyl CoA-related metabolite, a vitamin, histone deacetylase inhibitor (HDACi), a redox homeostasis regulator, a one carbon metabolism pathway intermediate, glutamate, and/or carnitine for a first period of 1, 2, 3, 4, 5, 6, or 7 days (e.g., 4 days). In some embodiments, the method further comprises contacting the population of cells following the first period with one or more of a serum albumin protein, an acetyl CoA-related metabolite, a vitamin, histone deacetylase inhibitor (HDACi), a redox homeostasis regulator, a one carbon metabolism pathway intermediate, glutamate, and/or carnitine for a second period of 1, 2, 3, 4, 5, 6, or 7 days (e.g., 3 days) or more in the absence of a TGF-β signaling pathway inhibitor, a TH signaling pathway activator, a protein kinase inhibitor, a ROCK inhibitor, a BMP signaling pathway inhibitor, and/or an epigenetic modifying compound. In some embodiments, the cells are contacted with the same concentration of the serum albumin (e.g., 0.05% HSA) in the second period as compared to the first period. In some embodiments, the cells are contacted with a higher concentration of the serum albumin in the second period as compared to the first period. In some embodiments, the compositions further comprise ZnSO₄. In some embodiments, the compositions further comprise DMEM/F12.

In some embodiments, the method comprises contacting the population of cells (e.g., NKX6.1-positive, ISL1-positive, insulin-positive cells) with one or more of HSA, Alk5 inhibitor II, GC-1, staurosporine, thiazovivin, LDN193189, DZNEP, taurine, acetate, betahydroxybutyrate, biotin, carnitine, glutamate, and formate for a first period of 1, 2, 3, 4, 5, 6, or 7 days (e.g., 4 days). In some embodiments, the method further comprises contacting the population of cells following the first period with one or more of HSA, taurine, acetate, betahydroxybutyrate, biotin, carnitine, glutamate, and formate for a second period of 1, 2, 3, 4, 5, 6, or 7 days (e.g., 3 days) or more in the absence of Alk5 inhibitor II, GC-1, staurosporine, thiazovivin, LDN193189, and DZNEP. In some embodiments, the compositions further comprise ZnSO₄. In some embodiments, the compositions further comprise DMEM/F12. In some embodiments, the cells are contacted with the same concentration of the serum albumin (e.g., 0.05% HSA) in the second period as compared to the first period. In some embodiments, the cells are contacted with a higher concentration of the HSA (e.g., about 1.0%) in the second period as compared to the first period (e.g., about 0.05%).

In some embodiments, compositions and methods disclosed herein improve cell recovery or yield after they are dissociated and subsequently reaggregated. Increased cell recovery can comprise an increased number or percentage of viable cells. Increased cell recovery can comprise an increased number or percentage of a population of interest (e.g., ISL1-positive and NKX6.1 positive cells or another population disclosed herein). In some embodiments, increased cell recovery is achieved after contacting the cells with a combination of agents disclosed herein. For example, in some embodiments, contacting cells with any one or more of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, and/or carnitine as disclosed herein increases cell recovery. In some embodiments, cell recovery is increased when the cells are contacted with the agents prior to dissociation. In some embodiments, cell recovery is increased when the cells are contacted with the agents after dissociation.

In some embodiments, at least 25%, at least 30%, at least 35%, at least 40%, at least 45%, at least 50%, at least 55%, or at least 60% of total viable cells present before dissociation are recovered as viable cells when evaluated after re-aggregation, for example, when evaluated after about 1 day, 2, days, 3, days, 4 days, 5 days, 6 days, 7 days, 8 days, 9 days, 10 days, 11 days, 12 days, 13 days, or 14 days after dissociation, or about 1 day, 2, days, 3, days, 4 days, 5 days, 6 days, 7 days, 8 days, 9 days, 10 days, 11 days, 12 days, 13 days, or 14 days after thawing dissociated and cryopreserved cells.

In some embodiments, the yield of total viable cells obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher compared to a method that not utilize an agent or combination of agents as disclosed herein.

In some embodiments, the yield of total viable cells obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher after re-aggregation compared to a method that not utilize an agent or combination of agents as disclosed herein, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells. In some embodiments, the yield of total viable cells is increased when the cells are contacted with an agent or combination of agents of the disclosure prior to dissociation of the earlier cluster. In some embodiments, the yield of total viable cells is increased when the cells are contacted with an agent or combination of agents of the disclosure after dissociation of the earlier cluster.

In some embodiments, at least 25%, at least 30%, at least 35%, at least 40%, at least 45%, at least 50%, at least 55%, or at least 60% of cells of a population of the disclosure (e.g., NKX6.1-positive and ISL1-positive cells, or another population disclosed herein) present before dissociation are recovered as viable cells when evaluated after re-aggregation, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells.

In some embodiments, the yield of cells of a population of the disclosure (e.g., NKX6.1-positive and ISL1-positive cells, or another population disclosed herein) obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher compared to a method that not utilize an agent or combination of agents as disclosed herein.

In some embodiments, the yield of cells of a population of the disclosure (e.g., NKX6.1-positive and ISL1-positive cells, or another population disclosed herein) obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher after re-aggregation compared to a method that not utilize an agent or combination of agents as disclosed herein, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells. In some embodiments, the yield is increased when the cells are contacted with the agent or combination of agents prior to dissociation of an earlier cluster. In some embodiments, the yield is increased when the cells are contacted with the agent or combination of agents after dissociation of the earlier cluster.

In some embodiments, compositions and methods disclosed herein increase the percent of cells in a cluster expressing both NKX6.1 and ISL1 after dissociation and reaggregation. For example, at least about 10%, 20%, 30%, 35%, 38%, 40%, 42%, 44%, 46%, 50%, 60%, 70%, 80%, 90%, 95%, or 99% cells in a cell cluster can express NKX6.1 and ISL1 after dissociation and reaggregation as disclosed herein. In some cases, at least about 35% cells in the cell cluster can express NKX6.1 and ISL1. In some cases, at least about 38% cells in the cell cluster can express NKX6.1 and ISL1. In some cases, at least about 40% cells in the cell cluster can express NKX6.1 and ISL1. In some cases, at least about 45% cells in the cell cluster can express NKX6.1 and ISL1. In some cases, at least about 50% cells in the cell cluster can express NKX6.1 and ISL1. In some embodiments, the percent of cells in a re-aggregated cluster expressing both NKX6.1 and ISL1 is increased when the cells are contacted with an agent or combination of agents of the disclosure prior to dissociation of an earlier cluster. In some embodiments, the percent of cells in a re-aggregated cluster is increased when the cells are contacted an agent or combination of agents of the disclosure after dissociation of an earlier cluster.

Dissociating of the first cell cluster can be performed using methods known in the art. Non-limiting exemplary methods for dissociating cell clusters include physical forces (e.g., mechanical dissociation such as cell scraper, trituration through a narrow bore pipette, fine needle aspiration, vortex disaggregation and forced filtration through a fine nylon or stainless steel mesh), enzymatic dissociation using enzymes such as trypsin, collagenase, TrypLE™, and the like, or a combination thereof. After dissociation, cells from the first cell cluster can be in a cell suspension, e.g., a single cell suspension. The term “suspension” as used herein can refer to cell culture conditions in which cells are not attached to a solid support. Cells proliferating in suspension can be stirred while proliferating using apparatus well known to those skilled in the art.

In some embodiments, the disclosure provides for a composition comprising dissociated cells. In some embodiments, the disclosure provides for a composition comprising a plurality of dissociated insulin-positive endocrine progenitor cells. In some embodiments, the dissociated cells are Ngn3-positive. In some embodiments, the dissociated cells are PDX.1 positive. In some embodiments, the dissociated cells are NKX6.1 positive. In some embodiments, the disclosure provides for a composition comprising dissociated cells (e.g., dissociated insulin-positive endocrine progenitor cells) and a BMP signaling pathway inhibitor. In some embodiments, the BMP signaling pathway inhibitor is LDN193189 or a derivative thereof. In some embodiments, the disclosure provides for a composition comprising a plurality of dissociated cells (e.g., dissociated insulin-positive endocrine progenitor cells) and a ROCK inhibitor. In some embodiments, the ROCK inhibitor is thiazovivin, Y-27632, Fasudil/HA1077, or 14-1152, or derivatives thereof. In some embodiments, the disclosure provides for a composition comprising a plurality of dissociated cells (e.g., dissociated insulin-positive endocrine progenitor cells) and a histone methyltransferase inhibitor. In some embodiments, the histone methyltransferase inhibitor is 3-Deazaneplanocin A hydrochloride, or a derivative thereof. In some embodiments, the disclosure provides for a composition comprising a plurality of dissociated cells (e.g., dissociated insulin-positive endocrine progenitor cells) and zinc. In some embodiments, the zinc is in the form of ZnSO₄. In some embodiments, the disclosure provides for a composition comprising a plurality of dissociated cells (e.g., dissociated insulin-positive endocrine progenitor cells) and a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a composition comprising a plurality of dissociated cells (e.g., dissociated insulin-positive endocrine progenitor cells) and a lipid. In some embodiments, the lipid is a saturated fatty acid. In some embodiments, the saturated fatty acid is palmitate. In some embodiments, the lipid is a unsaturated fatty acid. In some embodiments, the unsaturated fatty acid is oleic acid, linoleic acid, or palmitoleic acid.

Provided herein are compositions comprising isolated insulin-positive endocrine cells that have been contacted with an agent that inhibits expression or function of monoglyceride lipase (MGLL) in vitro and exhibit an increased ratio of monoglycerides to free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased ratio of free fatty acids to monoglycerides compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased level of free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit an increased level of monoglycerides compared to a corresponding population of isolated insulin-positive endocrine cells that have not been contacted with an agent that inhibits expression or function of monoglyceride lipase (MGLL) in vitro.

Provided herein are compositions comprising a population of insulin-positive endocrine cells and an agent that inhibits the conversion of monoglycerides to free fatty acids.

In some embodiments, the disclosure provides for a composition comprising a plurality of dissociated cells (e.g., dissociated insulin-positive endocrine progenitor cells) and glutamate. In some embodiments, the disclosure provides for a composition comprising a plurality of dissociated cells (e.g., dissociated insulin-positive endocrine progenitor cells) and an acetyl CoA-related metabolite (e.g., acetate). In some embodiments, the disclosure provides for a composition comprising a plurality of dissociated cells (e.g., dissociated insulin-positive endocrine progenitor cells) and a histone deacetylase inhibitor (HDACi; e.g., β-hydroxybutyrate). In some embodiments, the disclosure provides for a composition comprising a plurality of dissociated cells (e.g., dissociated insulin-positive endocrine progenitor cells) and L-carnitine. In some embodiments, the disclosure provides for a composition comprising a plurality of dissociated cells (e.g., dissociated insulin-positive endocrine progenitor cells) and glutamine (e.g., L-glutamine). In some embodiments, the disclosure provides for a composition comprising a plurality of dissociated cells (e.g., dissociated insulin-positive endocrine progenitor cells) and a redox homeostasis regulator (e.g., taurine). In some embodiments, the disclosure provides for a composition comprising a plurality of dissociated cells (e.g., dissociated insulin-positive endocrine progenitor cells) and a one carbon metabolism pathway intermediate (e.g., formate). In some embodiments, the disclosure provides for a composition comprising a plurality of dissociated cells (e.g., dissociated insulin-positive endocrine progenitor cells) and a vitamin (e.g., biotin). In some embodiments, the disclosure provides for a composition comprising a plurality of dissociated cells (e.g., dissociated insulin-positive endocrine progenitor cells) and DMEM/F12. In some embodiments, the disclosure provides for a composition comprising a plurality of dissociated cells (e.g., dissociated insulin-positive endocrine progenitor cells) and zinc (e.g., ZnSO₄). In some embodiments, the composition further comprises a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the composition comprises a TGF-β pathway inhibitor. In some embodiments, the TGF-β pathway inhibitor is Alk5i (SB505124), or a derivative thereof. In some embodiments, the composition comprises a thyroid hormone signaling pathway activator. In some embodiments, the thyroid hormone signaling pathway activator is GC-1 or T3, or a derivative thereof. In some embodiments, the composition comprises a ROCK inhibitor. In some embodiments, the ROCK inhibitor is thiazovivin. In some embodiments, the composition comprises a histone methyltransferase inhibitor. In some embodiments, the histone methyltransferase inhibitor is 3-Deazaneplanocin A hydrochloride. In some embodiments, the composition comprises a protein kinase inhibitor. In some embodiments, the protein kinase inhibitor is staurosporine. In some embodiments, the composition comprises vitamin C. In particular embodiments, the composition is in vitro. In some embodiments, the composition does not comprise a γ secretase inhibitor (e.g., XXI). In some embodiments, the dissociated insulin-positive endocrine progenitor cells were previously frozen.

In some embodiments, the disclosure provides for a composition comprising a plurality of cell clusters. In some embodiments, the disclosure provides for a composition comprising a plurality of cell clusters; wherein the cell clusters comprise insulin-positive cells; wherein at least 30%, at least 35%, at least 40%, at least 45%, at least 50%, at least 55%, at least 60%, or at least 65% of the cells in the composition are viable following 11 days in culture in vitro.

In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter.

In some embodiments, the disclosure provides for a composition comprising a plurality of cell clusters; wherein the cell clusters comprise insulin-positive cells; wherein at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm.

In some embodiments, the disclosure provides for a composition comprising a plurality of cell clusters; wherein the cell clusters comprise insulin-positive cells; wherein the cell clusters have a mean or median diameter of at most 120, at most 130, at most 140, at most 150, at most 160, or at most 170 μm.

In some embodiments, the disclosure provides for a composition comprising a plurality of cell clusters; wherein the cell clusters comprise insulin-positive cells; wherein the cell clusters have a mean or median diameter of about 80-150, about 100-150, about 120-150, about 140-150, about 80-130, about 100-130, about 120-130, about 80-120, about 90-120, or about 100-120 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 150 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 140 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 150 μm.

In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, the cell clusters comprise C-peptide positive cells. In some embodiments, the cell clusters comprise somatostatin positive cells. In some embodiments, the cell clusters comprise glucagon positive cells. In some embodiments, at least 30%, at least 35%, at least 40%, at least 45%, at least 50%, at least 55%, at least 60%, or at least 65% of the cells in the composition are viable following 11 days in culture in vitro.

In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter.

In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, at least 2, 3, 4, 5, 10, 50, 100, 1000, 10000, 100000, or 1000000 cell clusters. In some embodiments, the composition is prepared in accordance with any of the methods disclosed herein. In some embodiments, the disclosure provides for a device comprising the any of the cell compositions disclosed herein. In some embodiments, the disclosure provides for a method of treating a subject with a disease characterized by high blood sugar levels over a prolonged period of time (e.g., diabetes), the method comprising administering any of the compositions disclosed herein or any of the devices disclosed herein to the subject.

In some embodiments, the disclosure provides for a method comprising the step of contacting a plurality of dissociated insulin-positive endocrine progenitor cells with a BMP signaling pathway inhibitor. In some embodiments, the BMP signaling pathway inhibitor is LDN193189 or a derivative thereof. In some embodiments, the disclosure provides for a method comprising the step of contacting a plurality of dissociated insulin-positive endocrine progenitor cells with a ROCK inhibitor. In some embodiments, the ROCK inhibitor is thiazovivin, Y-27632, Fasudil/HA1077, or 14-1152, or derivatives thereof. In some embodiments, the disclosure provides for a method comprising the step of contacting a plurality of dissociated insulin-positive endocrine progenitor cells with a histone methyltransferase inhibitor. In some embodiments, the histone methyltransferase inhibitor is 3-Deazaneplanocin A hydrochloride, or a derivative thereof. In some embodiments, the disclosure provides for a method comprising the step of contacting a plurality of dissociated insulin-positive endocrine progenitor cells with zinc. In some embodiments, the zinc is in the form of ZnSO₄. In some embodiments, the disclosure provides for a method comprising the step of contacting a plurality of dissociated insulin-positive endocrine progenitor cells with a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a method comprising the step of contacting a plurality of dissociated insulin-positive endocrine progenitor cells with a lipid. In some embodiments, the lipid is a saturated fatty acid. In some embodiments, the saturated fatty acid is palmitate. In some embodiments, the lipid is an unsaturated fatty acid. In some embodiments, the unsaturated fatty acid is oleic acid, linoleic acid, or palmitoleic acid.

In some embodiments, the disclosure provides for a method comprising the step of contacting a plurality of dissociated insulin-positive endocrine progenitor cells with glutamate. In some embodiments, the disclosure provides for a method comprising the step of contacting a plurality of dissociated insulin-positive endocrine progenitor cells with an acetyl CoA-related metabolite (e.g., acetate). In some embodiments, the disclosure provides for a method comprising the step of contacting a plurality of dissociated insulin-positive endocrine progenitor cells with an HDAC inhibitor (e.g., β-hydroxybutyrate). In some embodiments, the disclosure provides for a method comprising the step of contacting a plurality of dissociated insulin-positive endocrine progenitor cells with L-carnitine. In some embodiments, the disclosure provides for a method comprising the step of contacting a plurality of dissociated insulin-positive endocrine progenitor cells with L-glutamine. In some embodiments, the disclosure provides for a method comprising the step of contacting a plurality of dissociated insulin-positive endocrine progenitor cells with a redox homeostasis regulator (e.g., taurine). In some embodiments, the disclosure provides for a method comprising the step of contacting a plurality of dissociated insulin-positive endocrine progenitor cells with a one carbon metabolism pathway intermediate (e.g., formate). In some embodiments, the disclosure provides for a method comprising the step of contacting a plurality of dissociated insulin-positive endocrine progenitor cells with a vitamin (e.g., biotin). In some embodiments, the method comprises contacting the plurality of dissociated insulin-positive endocrine progenitor cells with a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the method comprises contacting the plurality of dissociated insulin-positive endocrine progenitor cells with a TGF-β pathway inhibitor. In some embodiments, the TGF-β pathway inhibitor is Alk5i (SB505124), or a derivative thereof. In some embodiments, the method comprises contacting the plurality of dissociated insulin-positive endocrine progenitor cells with a thyroid hormone signaling pathway activator. In some embodiments, the thyroid hormone signaling pathway activator is GC-1 or T3, or a derivative thereof. In some embodiments, the method comprises contacting the plurality of dissociated insulin-positive endocrine progenitor cells with a protein kinase inhibitor. In some embodiments, the protein kinase inhibitor is staurosporine. In some embodiments, the method comprises contacting the plurality of dissociated insulin-positive endocrine progenitor cells with vitamin C. In some embodiments, the method does not comprise the step of contacting the plurality of dissociated insulin-positive endocrine cells with a γ secretase inhibitor (e.g., XXI). In some embodiments, the dissociated insulin-positive endocrine progenitor cells were previously frozen. In some embodiments, the method is performed over the course of 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days. In some embodiments, the method results in the reaggregation of the dissociated cells into a plurality of cell clusters. In some embodiments, at least about 40%, 50%, 60%, 70%, 80%, or 90% of the plurality of cell clusters have a diameter from about 50 μm to about 250 μm, from about 75 μm to about 250 μm, or from about 100 μm to about 200 μm. In some embodiments, at least about 40%, 50%, 60%, 65%, 70%, 75%, 80%, 85%, 95%, or 99% of the cells of the plurality of cell clusters of the second cell population are viable. In some embodiments, the method results in the reaggregation of the dissociated cells into at least 2, 3, 4, 5, 10, 50, 100, 1000, 10000, 100000, or 1000000 cell clusters.

In some embodiments, any of the cells disclosed herein comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, any of the cells disclosed herein (e.g., any of the SC-derived beta cells or cells in any of the clusters disclosed herein) comprise a genomic disruption in at least one gene sequence, wherein said disruption reduces or eliminates expression of a protein encoded by said gene sequence. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLAB, but do not comprise a genomic disruption in the gene encoding HLA-C. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen-related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to cells that are not genetically modified. In some embodiments, the cells have increased expression of CD47, PDL1, HLA-G, CD46, CD55, CD59 and CTLA, relative to cells that are not genetically modified. In particular embodiments, the pancreatic islet cells disclosed herein (e.g., the SC-beta cells) have increased expression of PDL1 as compared to endogenous pancreatic islet cells from a healthy control subject. In particular embodiments, the pancreatic islet cells disclosed herein (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology.

In some cases, the method provided herein does not comprise an active cell sorting process, e.g., flow cytometry. In some cases, a cell cluster as described herein can be an unsorted cell cluster. In some cases, a method provided herein does not rely on an active cell sorting for the enrichment or elimination of a particular type of cells in the first cell cluster. In some cases, a method merely requires dissociating the first cell cluster and culturing the plurality of cells dissociated from the first cell cluster in a medium, thereby allowing formation of a second cell cluster.

In some cases, the method provided herein can be applied to dissociate a cell cluster and reaggregate into a new cluster for more than once. For instance, a first cell cluster can be dissociated and reaggregated to form a second cell cluster according to the method provided herein, and the second cell cluster can be further dissociated and reaggregated to form a third cell cluster, and so on. Reaggregation as provided herein can be performed sequentially to a cell cluster for at least 1, 2, 3, 4, 5, 6, 7, 8, 9, or 10 times.

Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells' optical properties (e.g., emission wave length upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. An unsorted cell cluster, in some cases referred to as “reaggregated cell cluster,” can be formed by a plurality of cells that are dissociated from an existing cell cluster, and before their reaggregation into the new cell cluster, there can be no active cell sorting process, e.g., flow cytometry or other methods, to isolate one or more particular cell types for the reaggregation as provided herein. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in said cells. The GFP signal can thus be a marker of a pancreatic β cell. In some cases, cell sorting as described herein can comprise magnetic-activated flow cytometry, where magnetic antibody or other ligand is used to label cells of different types, and the differences in magnetic properties can be used for cell sorting.

The cells dissociated from the first cell cluster can be cultured in a medium for re-aggregating to a second cell cluster. The medium can comprise Connought Medical Research Laboratories 1066 supplemented islet media (CMRLS). In some cases, the suitable culture medium comprises a component of CMRLS (e.g., supplemental zinc). The CMRLS can be supplemented, e.g., with serum (e.g., human serum, human platelet lysate, fetal bovine serum, or serum replacements such as Knockout Serum Replacement).

The medium can comprise one or more compounds that regulate certain signaling pathways in cells. For example, the medium can comprise a thyroid hormone signaling pathway activator, a transforming growth factor β (TGF-0) signaling pathway inhibitor, or both.

The thyroid hormone signaling pathway activator in the medium used herein can be triiodothyronine (T3). In some cases, the thyroid hormone signaling pathway activator can be an analog or derivative of T3. Non-limiting exemplary analogs of T3 include selective and non-selective thyromimetics, TRO selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine (T4) and triiodothyronine (T3) alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA). In some cases, the thyroid hormone signaling pathway activator is a prodrug or prohormone of T3, such as T4 thyroid hormone (e.g., thyroxine or L-3,5,3′,5′-tetraiodothyronine). The thyroid hormone signaling pathway activator can also be an iodothyronine composition described in U.S. Pat. No. 7,163,918, which is incorporated by reference herein in its entirety.

The concentration of the thyroid hormone signaling pathway activator in the medium can be in a range suitable for cell aggregation. In some cases, the concentration of the thyroid hormone signaling pathway activator in the medium is from about 0.1 μM to about 10 μM, such as from about 0.5 μM to about 2 μM, from about 0.8 μM to about 1.5 μM, from about 0.9 μM to about 1.5 μM, from about 0.9 μM to about 1.2 μM, or from about 0.9 μM to about 1.2 μM. In some cases, the contraction of the thyroid hormone signaling pathway activator in the medium is at least about 0.1 μM, 0.2 μM, 0.4 μM, 0.8 μM, 0.9 μM, 1 μM, 1.1 μM, 1.2 μM, 1.3 μM, 1.4 μM, 1.5 μM, 2 μM, 3 μM, 4 μM, 5 μM, 6 μM, 7 μM, 8 μM, 9 μM, or 10 μM. In some case, the contraction of the thyroid hormone signaling pathway activator (e.g., T3) in the medium is about 1 μM.

The TGF-β signaling pathway inhibitor used in the medium herein can be an inhibitor of TGF-β receptor type I kinase (TGF-β RI) signaling. The TGF-β signaling pathway inhibitor can be an activin receptor-like kinase-5 (Alk5) inhibitor, e.g., ALK5 inhibitor II (CAS 446859-33-2, an ATP-competitive inhibitor of TGF-β RI kinase, also known as RepSox, IUPAC Name: 2-[5-(6-methylpyridin-2-yl)-1H-pyrazol-4-yl]-1,5-naphthyridine). In some cases, the TGF-β signaling pathway inhibitor is an analog or derivative of ALK5 inhibitor II, including those described in in U.S. Patent Publication Nos. 2012/0021519, 2010/0267731, 2009/0186076, and 2007/0142376, which are incorporated by reference herein in their entireties. In some cases, examples of TGF-β signaling pathway inhibitor that can be used in the medium herein also include D 4476, SB431542, A-83-01, also known as 3-(6-Methyl-2-pyridinyl)-N-phenyl-4-(4-quinolinyl)-1H-pyrazole-1-carbothioamide; 2-(3-(6-Methylpyridin-2-yl)-1H-pyrazol-4-yl)-1, 5-naphthyridine, Wnt3a/BIO, BMP4, GW788388 (-(4-[3-(pyridin-2-yl)-1H-pyrazol-4-yl]pyridin-2-yl}-N-(tetrahydro-2H-pyran-4-yl)benzamide), SMI 6, ΓN-1130 (3-((5-(6-methylpyridin-2-yl)-4-(quinoxalin-6-yl)-1H-imidazol-2-yl)methyl)benzamide, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), SU5416, lerdelimumab (CAT-152), metelimumab (CAT-192), GC-1008, ID11, AP-12009, AP-1 1014, LY550410, LY580276, LY364947, LY2109761, SD-208, SM16, NPC-30345, KÏ26894, SB-203580, SD-093, ALX-270-448, EW-7195, SB-525334, ΓN-1233, SKI2162, Gleevec, 3,5,7,2′,4′-pentahydroxyfiavone (Morin), activin-M108A, P144, soluble TBR2-Fc, pyrimidine derivatives and indolinones. Inhibition of the TGF-β/activin pathway can have similar effects. Thus, any inhibitor (e.g., upstream or downstream) of the TGF-β/activin pathway can be used in combination with, or instead of, TGF-β/ALK5 inhibitors as described herein. Exemplary TGF-β/activin pathway inhibitors include, but are not limited to, TGF-β receptor inhibitors, inhibitors of SMAD 2/3 phosphorylation, inhibitors of the interaction of SMAD 2/3 and SMAD 4, and activators/agonists of SMAD 6 and SMAD 7. Furthermore, the categorizations described herein are merely for organizational purposes and one of skill in the art would know that compounds can affect one or more points within a pathway, and thus compounds may function in more than one of the defined categories. TGF-β receptor inhibitors may include any inhibitors of TGF signaling in general or inhibitors specific for TGF-β receptor (e.g., ALK5) inhibitors, which can include antibodies to, dominant negative variants of, and siRNA and antisense nucleic acids that suppress expression of, TGF-β receptors.

The concentration of the TGF-β signaling pathway inhibitor in the medium can be in a range suitable for cell aggregation. In some cases, the concentration of the TGF-β signaling pathway inhibitor in the medium is from about 1 μM to about 50 μM, such as from about 5 μM to about 15 μM, from about 8 μM to about 12 μM, or from about 9 μM to about 11 μM. In some cases, the contraction of the TGF-β signaling pathway inhibitor in the medium is at least about 1 μM, 5 μM, 8 μM, 9 μM, 10 μM, 11 μM, 12 μM, 13 μM, 14 μM, 15 μM, 20 μM, 25 μM, 30 μM, 35 μM, 40 μM, 45 μM, or 50 μM. In some case, the contraction of the TGF-β signaling pathway inhibitor (e.g., Alk5 inhibitor II) in the medium is about 10 μM.

The medium used to culture the cells dissociated from the first cell cluster can be xeno-free. A xeno-free medium for culturing cells and/or cell clusters of originated from an animal can have no product from other animals. In some cases, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some cases, medium can comprise about 10% of serum. In some cases, the medium can be free of small molecules and/or FBS. For example, a medium can comprise MCDB131 basal medium supplemented with 2% BSA. In some cases, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant 4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate 13-acetate, bryostatin 1, or derivatives thereof), antagonist of TGF beta super family (e.g., Alk5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenic Protein (BMP) type 1 receptor (e.g., LDN193189 or derivatives thereof), thyroid hormone signaling pathway activator (e.g., T3 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some cases, the reaggregation medium can comprise no exogenous extracellular matrix molecule. In some cases, the reaggregation medium does not comprise Matrigel™. In some cases, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that that the concentration of BSA supplemented into the medium may vary. For example, a medium (e.g., MCDB131) can comprise about 0.01%, 0.05%, 0.1%, 1%, about 2%, about 3%, about 4%, about 5%, about 10%, or about 15% BSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine.

The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some cases, the medium comprises laminin, such as LN-332. In some cases, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

Cells dissociated from the first cell cluster can be seeded in a container for re-aggregation. The seeding density can correlate with the size of the re-aggregated second cell cluster. The seeding density can be controlled so that the size of the second cell cluster can be similar to an endogenous pancreatic islet. In some cases, the seeding density is controlled so that the size of the second cell cluster can be from about 75 μm to about 250 μm. Cells dissociated from the first cell cluster can be seeded at a density of from about 0.1 million cells per mL to about 10 million cells per mL, e.g., from about 0.5 million cells per mL to about 1.5 million cells per mL, from about 0.8 million cells per mL to about 1.2 million cells per mL, from about 0.9 million cells per mL to about 1.1 million cells per mL, from about 2 million cells per mL to about 3 million cells per mL. In some cases, the cells dissociated from the first cell cluster can be seeded at a density of about 1 million cells per mL. In some cases, the cells dissociated from the first cell cluster can be seeded at a density of about 1.5 million cells per mL. In some cases, the cells dissociated from the first cell cluster can be seeded at a density of about 2 million cells per mL. In some cases, the cells dissociated from the first cell cluster can be seeded at a density of about 2.5 million cells per mL. In some cases, the cells dissociated from the first cell cluster can be seeded at a density of about 3 million cells per mL.

The cells dissociated from the first cell cluster can be cultured in a culture vessel. The culture vessel can be suitable for culturing a suspension of culture of cells. The culture vessel used for culturing the cells or cell clusters herein can include, but is not limited to: flask, flask for tissue culture, dish, petri dish, dish for tissue culture, multi dish, micro plate, micro-well plate, multi plate, multi-well plate, micro slide, chamber slide, tube, tray, culture bag, and roller bottle, stir tank bioreactors, or polymer (e.g., biopolymer or gel) encapsulation as long as it is capable of culturing the cells therein. The cells and/or cell clusters can be cultured in a volume of at least or about 0.2 ml, 0.5 ml, 1 ml, 5 ml, 10 ml, 20 ml, 30 ml, 40 ml, 50 ml, 100 ml, 150 ml, 200 ml, 250 ml, 300 ml, 350 ml, 400 ml, 450 ml, 500 ml, 600 ml, 800 ml, 1000 ml, 1500 ml, 2000 ml, 3000 ml or any range derivable therein, depending on the needs of the culture.

In some cases, cells can be cultured under dynamic conditions (e.g., under conditions in which the cells are subject to constant movement or stirring while in the suspension culture). For dynamic culturing of cells, the cells can be cultured in a container (e.g., an non-adhesive container such as a spinner flask (e.g., of 200 ml to 3000 ml, for example 250 ml; of 100 ml; or in 125 ml Erlenmeyer), which can be connected to a control unit and thus present a controlled culturing system. In some cases, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

Medium in a dynamic cell culture vessel (e.g., a spinner flask) can be stirred (e.g., by a stirrer). The spinning speed can correlate with the size of the re-aggregated second cell cluster. The spinning speed can be controlled so that the size of the second cell cluster can be similar to an endogenous pancreatic islet. In some cases, the spinning speed is controlled so that the size of the second cell cluster can be from about 75 μm to about 250 μm. The spinning speed of a dynamic cell culture vessel (e.g., a spinner flask) can be about 20 rounds per minute (rpm) to about 100 rpm, e.g., from about 30 rpm to about 90 rpm, from about 40 rpm to about 60 rpm, from about 45 rpm to about 50 rpm. In some cases, the spinning speed can be about 50 rpm.

The cells dissociated from the first cell cluster can be cultured for a period of time to allow them for re-aggregating. The cells dissociated from the first cell cluster can be cultured for at least 12 hours, 24 hours, 1 day, 2 days, 3 days, 4 days, 5 days, 6 days, 7 days 8 days, 9 days 10 days, 15 days, 20 days, 25 days, or 30 days. In some cases, the cells dissociated from the first cell cluster can be cultured for at least 4 days.

The methods herein can also be used to enrich cells resembling endogenous cells, e.g., endogenous mature pancreatic β cells in a cell cluster. The methods can comprise dissociating a first cell cluster and re-aggregating the cells from the first cluster to a second cluster. The second cluster can comprise more cells resembling endogenous mature pancreatic β cells compared to the first cluster. The dissociating and re-aggregating can be performed using any methods and reagents disclosed through the application.

After re-aggregation, the second cell cluster can comprise more cells expressing one or more markers of an endogenous cell compared to the first cell cluster. For example, the second cluster can comprise more cells expressing one or more markers of an endogenous mature pancreatic β cell, the markers including insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN, compared to the first cell cluster. In some cases, the second cluster can comprise more cells expressing CHGA. In some cases, the second cluster can comprise more cells expressing NKX6.1. In some cases, the second cluster can comprise more cells expressing C-peptide. In some cases, the second cluster can comprise more cells expressing NKX6.1 and C-peptide. In some cases, the second cluster can comprise more cells expressing CHGA, NKX6.1 and C-peptide.

After re-aggregation, the second cell cluster can have a smaller size (e.g., a smaller diameter) compared to the first cell cluster. The smaller size can allow better exchange of molecules between the cell cluster and the surrounding environment. For example, a smaller size can allow better diffusion of molecules (e.g., reagents, gas, and/or nutrition) from the medium to the cells in a cell cluster. Thus, being in a smaller size, the second cell cluster can exchange molecules with the surrounding environment in a more efficient way compared to the first cell cluster. Thus the second cell cluster can have less dead cells (e.g., cells died due to insufficient nutrition and/or gas) compared to the first cell cluster.

A method provided herein can enrich endocrine cells, e.g., cells expressing chromogranin A (CHGA). For examples, a percentage of cells in the second cell cluster that express chromogranin A is at least 1.2, at least 1.3, at least 1.4, or at least 1.5 times more than a percentage of cells in the first cell cluster that express chromogranin A. In some cases, the second cell cluster comprises at least about 70%, at least about 80%, at least about 90%, at least about 95%, at least about 99%, or 100% cells expressing CHGA. In some cases, at least about 85% cells in the second cell cluster can express CHGA. In some cases, the second cell cluster can comprise about 90% cell expressing CHGA. In some cases, the second cell cluster can comprise about 95% cells expressing CHGA. In certain cases, all cells in the second cell cluster can express CHGA.

A method provided herein can generate or enrich pancreatic β cell. For example, the second cell cluster comprises at least one pancreatic β cell, e.g., at least one non-native pancreatic β cell. For examples, a percentage of cells in the second cell cluster that express both NKX6.1 and C-peptide is at least 1.5, at least 1.75, or at least 2 times more than a percentage of cells in the first cell cluster that express both NKX6.1 and C-peptide. In some cases, the second cell cluster comprises at least about 35%, at least about 40%, at least about 50%, at least about 60%, at least about 70%, at least about 80%, at least about 90%, at least about 95%, at least about 99%, or 100% cells expressing NKX6.1 and C-peptide. In some cases, at least about 35% cells in the second cell cluster can express NKX6.1 and C-peptide. In some cases, a cell cluster can comprise about 60% cells expressing NKX6.1 and C-peptide. In some cases, the second cell cluster can comprise about 75% cell expressing NKX6.1 and C-peptide. In some cases, all cells in the second cell cluster can express NKX6.1 and C-peptide. In some cases, at least about 70% of the at least one non-native pancreatic β cell in the second cell cluster express chromogranin A as measured by flow cytometry. In some cases, at least about 25% of the at least one non-native pancreatic β cell in the second cell cluster express NKX6.1 and C-peptide as measured by flow cytometry.

A method provided herein can reduce or eliminate stem cells or precursor cells of a pancreatic endocrine cell. In some cases, a percentage of cells in the second cell cluster that express SOX2 is at least 2, at least 3, at least 5, or at least 10 times lower than a percentage of cells in the first cell cluster that express LIN28, Ki67, SOX2, or SOX9. For example, the second cell cluster can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, the second cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67. For example, the second cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, the second cell cluster can comprise about 1% cells expressing SOX2. In some cases, the second cell cluster can comprise about 0.6% cells expressing SOX2. In some cases, the second cell cluster can comprise about 0.3% cells expressing SOX2. In some cases, the second cell cluster can comprise about 0.1% cells expressing SOX2. For examples, the second cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, the second cell cluster can comprise about 2% cells expressing SOX9. In some cases, the second cell cluster can comprise about 6% cells expressing SOX9. In some cases, the second cell cluster can comprise about 1.2% cells expressing SOX9.

The second cell cluster can also function more similarly to an endogenous pancreatic islet compared to the first cell cluster. The second cell cluster can have a higher insulin content than the first cell cluster, for instance, at least 1.1, at least 1.25 or at least 1.5 times higher insulin content as compared to the first cell cluster. The second cluster can exhibit a greater in vitro GSIS than the first cell cluster, as measured by stimulation indexes. The second cluster can also exhibit a greater in vivo GSIS than the first cell cluster, as measured by stimulation indexes. In some cases, the second cluster can exhibit a greater in vitro GSIS and a greater in vivo GSIS compared to the first cell cluster, as measured by stimulation indexes. For example, the second cell cluster can secrete more insulin than the first cell cluster under the same stimulation conditions. The second cell cluster can also exhibit insulin secretion response to a potassium challenge (K⁺), e.g., a concentration of KCl, e.g., 30 mM KCl.

In some cases, the method provided herein can retain a large percentage of cells from the first cell cluster in the second cell cluster, e.g., pancreatic β cells or endocrine cells. For example, at least about 95%, at least about 98%, or at least about 99% of cells that express both NKX6.1 and C-peptide in the first cell cluster can be retained in the second in vitro cell cluster. In some cases, at most about 5%, at most about 2%, at most about 1%, at most about 0.5%, or at most about 0.1% of cells that express both NKX6.1 and C-peptide in the first cell cluster are lost during the dissociation and reaggregation process.

In some embodiments, a method provided herein provides a population of SC-β cells with increased stability or shelf life. For example, in some embodiments, a method of the discourse provides a population of cells that has at least 15%, at least 20%, at least 25%, at least 20%, at least 35%, at least 38%, at least 40%, at least 45%, or at least 50% ISL1-positive, NKX6.1-positive cells after 4 days, 7 days, or 10 days in culture.

In some embodiments, increased stability as achieved based on contacting the cells with a combination of agents disclosed herein. For example, in some embodiments, contacting cells with any one or more of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, and/or carnitine as disclosed herein increases the number or relative proportion of cells that are ISL1-positive and NKX6.1 positive after 3, 4, 5, 6, 7, 8, 9, 10, 11, 12, 13, or 14 days in cell culture media.

In some cases, the cell cluster as described herein is generated from any starting cell population in vitro. For example, the starting cell can include, without limitation, insulin-positive endocrine cells (e.g., chromogranin A-positive cells) or any precursor thereof, such as a Nkx6.1-positive pancreatic progenitor cell, a Pdx1-positive pancreatic progenitor cell, and a pluripotent stem cell, an embryonic stem cell, and induced pluripotent stern cell. In some cases, the method include differentiation of a reprogrammed cell, a partially reprogrammed cell (e.g., a somatic cell, e.g., a fibroblast which has been partially reprogrammed such that it exists in an intermediate state between an induced pluripotency cell and the somatic cell from which it has been derived), a transdifferentiated cell. In some cases, the cell cluster comprising the pancreatic β cell disclosed herein can be differentiated in vitro from an insulin-positive endocrine cell or a precursor thereof. In some cases, the cell cluster comprising the pancreatic β cell is differentiated in vitro from a precursor selected from the group consisting of a NKX6.1-positive pancreatic progenitor cell, a Pdx1-positive pancreatic progenitor cell, and a pluripotent stem cell. In some cases, the pluripotent stem cell is selected from the group consisting of an embryonic stem cell and induced pluripotent stem cell. As discussed above, the non-native pancreatic β cells can also be referred to as stem cell-derived β cells (SC-β cells) as they can be derived from stem cells in vitro. In some cases, the SC-β cell or the pluripotent stem cell from which the SC-β cell is derived is human. In some cases, the SC-β cell is human.

One aspect of the present disclosure provides a method of generating non-native pancreatic β cells. In some cases, the method can be any currently available protocol, such as those described in U.S. patent application Ser. Nos. 14/684,129; 14/684,101; 17/390,839 and 17/390,839 (e.g., Version A from Example 1), each of which is incorporated herein by its entirety. Aspects of the disclosure involve definitive endoderm cells. Definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), Pdx1-positive pancreatic progenitor cells (stage 3), NKX6.1-positive pancreatic progenitor cells (stage 4), or Ngn3-positive endocrine progenitor cells or insulin-positive endocrine cells (stage 5), followed by induction or maturation to SC-β cells (stage 6).

In some cases, definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

Any growth factor from the TGF-β superfamily capable of inducing the pluripotent stem cells to differentiate into definitive endoderm cells (e.g., alone, or in combination with a WNT signaling pathway activator) can be used in the method provided herein. In some cases, the at least one growth factor from the TGF-β superfamily comprises Activin A. In some cases, the at least one growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily) can be used in the method provided herein. In some cases, the WNT signaling pathway activator comprises CHIR99021. In some cases, the WNT signaling pathway activator comprises Wnt3a recombinant protein.

In some cases, differentiating at least some pluripotent cells in a population into definitive endoderm cells is achieved by a process of contacting a population of pluripotent cells with i) Activin A, and ii) CHIR99021 for a period of 3 days, to induce the differentiation of at least some of the pluripotent cells in the population into definitive endoderm cells, wherein the definitive endoderm cells express at least one marker characteristic of definitive endoderm. In some cases, differentiating at least some pluripotent cells in a population into definitive endoderm cells is achieved by a process of contacting a population of pluripotent cells with i) Activin A, and ii) CHIR99021 for 1 day, followed by contacting the population with activin A (in the absence of CHIR99021) for 2 days.

In some cases, a definitive endoderm cell produced by the methods as disclosed herein expresses at least one marker selected from the group consisting of: Nodal, Tmprss2, Tmem30b, St14, Spink3, Sh3g12, Ripk4, Rab1S, Npnt, Clic6, CldnS, Cacna1b, Bnip1, Anxa4, Emb, FoxA1, Sox17, and Rbm35a, wherein the expression of at least one marker is upregulated to by a statistically significant amount in the definitive endoderm cell relative to the pluripotent stem cell from which it was derived. In some cases, a definitive endoderm cell produced by the methods as disclosed herein does not express by a statistically significant amount at least one marker selected the group consisting of: Gata4, SPARC, AFP and Dab2 relative to the pluripotent stem cell from which it was derived. In some cases, a definitive endoderm cell produced by the methods as disclosed herein does not express by a statistically significant amount at least one marker selected the group consisting of: Zic1, Pax6, Flk1 and CD31 relative to the pluripotent stem cell from which it was derived. In some cases, a definitive endoderm cell produced by the methods as disclosed herein has a higher level of phosphorylation of Smad2 by a statistically significant amount relative to the pluripotent stem cell from which it was derived. In some cases, a definitive endoderm cell produced by the methods as disclosed herein has the capacity to form gut tube in vivo. In some cases, a definitive endoderm cell produced by the methods as disclosed herein can differentiate into a cell with morphology characteristic of a gut cell, and wherein a cell with morphology characteristic of a gut cell expresses FoxA2 and/or Claudin6, In some cases, a definitive endoderm cell produced by the methods as disclosed herein can be further differentiated into a cell of endoderm origin.

In some cases, a population of pluripotent stem cells are cultured in the presence of at least one β cell maturation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some cases, a β cell maturation factor as described herein can be present in the culture medium of a population of pluripotent stem cells or may be added in bolus or periodically during growth (e.g. replication or propagation) of the population of pluripotent stem cells. In certain examples, a population of pluripotent stem cells can be exposed to at least one β cell maturation factor prior to any differentiation. In other examples, a population of pluripotent stem cells may be exposed to at least one β cell maturation factor during the first stage of differentiation.

Aspects of the disclosure involve primitive gut tube cells. Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to Pdx1-positive pancreatic progenitor cells, NKX6.1-positive pancreatic progenitor cells, Ngn3-positive endocrine progenitor cells, insulin-positive endocrine cells, followed by induction or maturation to SC-β cells.

In some cases, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

Any growth factor from the FGF family capable of inducing definitive endoderm cells to differentiate into primitive gut tube cells (e.g., alone, or in combination with other factors) can be used in the method provided herein. In some cases, the at least one growth factor from the FGF family comprises keratinocyte growth factor (KGF). In some cases, the at least one growth factor from the FGF family comprises FGF2. In some cases, the at least one growth factor from the FGF family comprises FGF8B. In some cases, the at least one growth factor from the FGF family comprises FGF 10. In some cases, the at least one growth factor from the FGF family comprises FGF21.

In some cases, primitive gut tube cells can be obtained by differentiating at least some definitive endoderm cells in a population into primitive gut tube cells, e.g., by contacting definitive endoderm cells with KGF for a period of 2 or 3 days, to induce the differentiation of at least some of the definitive endoderm cells into primitive gut tube cells.

Aspects of the disclosure involve Pdx1-positive pancreatic progenitor cells. Pdx1-positive pancreatic progenitor cells of use herein can be derived from any source or generated in accordance with any suitable protocol. In some aspects, primitive gut tube cells are differentiated to Pdx1-positive pancreatic progenitor cells. In some aspects, the Pdx1-positive pancreatic progenitor cells are further differentiated, e.g., NKX6.1-positive pancreatic progenitor cells, Ngn3-positive endocrine progenitor cells, insulin-positive endocrine cells, followed by induction or maturation to SC-β cells,

In some aspects, Pdx1-positive pancreatic progenitor cells can be obtained by differentiating at least some primitive gut tube cells in a population into Pdx1-positive pancreatic progenitor cells, e.g., by contacting primitive gut tube cells with i) at least one bone morphogenic protein (BMP) signaling pathway inhibitor, ii) at least one growth factor from the FGF family, in) at least one SHH pathway inhibitor, iv) at least one retinoic acid (RA) signaling pathway activator; and v) at least one protein kinase C activator, to induce the differentiation of at least some of the primitive gut tube cells into Pdx1-positive pancreatic progenitor cells, wherein the Pdx1-positive pancreatic progenitor cells express Pdx1. In some cases, Pdx1-positive pancreatic progenitor cells can be obtained by differentiating at least some primitive gut tube cells in a population into Pdx1-positive pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into Pdx1-positive pancreatic progenitor cells, wherein the Pdx1-positive pancreatic progenitor cells express Pdx1.

Any BMP signaling pathway inhibitor capable of inducing primitive gut tube cells to differentiate into Pdx1-positive pancreatic progenitor cells (e.g., alone, or with any combination of at least one growth factor from the FGF family, at least one SHH pathway inhibitor, at least one retinoic acid signaling pathway activator, and at least one protein kinase C activator) can be used in the method provided herein. In some cases, the BMP signaling pathway inhibitor comprises LDN193189.

Any growth factor from the FGF family capable of inducing primitive gut tube cells to differentiate into Pdx1-positive pancreatic progenitor cells (e.g., alone, or with any combination of at least one BMP signaling pathway inhibitor, at least one SHH pathway inhibitor, at least one retinoic acid signaling pathway activator, and at least one protein kinase C activator) can be used. In some cases, the at least one growth factor from the FGF family comprises keratinocyte growth factor (KGF). In some cases, the at least one growth factor from the FGF family is selected from the group consisting of FGF2, FGF8B, FGF10, and FGF21.

Any SHH pathway inhibitor capable of inducing primitive gut tube cells to differentiate into Pdx1-positive pancreatic progenitor cells (e.g., alone, or with any combination of at least one BMP signaling pathway inhibitor, at least one growth factor from the FGF family, at least one retinoic acid signaling pathway activator, and at least one protein kinase C activator) can be used. In some cases, the SHH pathway inhibitor comprises Sant 1.

Any RA signaling pathway activator capable of inducing primitive gut tube cells to differentiate into Pdx1-positive pancreatic progenitor cells (e.g., alone, or with any combination of at least one BMP signaling pathway inhibitor, at least one growth factor from the FGF family, at least one SHH pathway inhibitor, and at least one protein kinase C activator) can be used. In some cases, the RA signaling pathway activator comprises retinoic acid.

Any PKC activator capable of inducing primitive gut tube cells to differentiate into Pdx1-positive pancreatic progenitor cells (e.g., alone, or with any combination of at least one BMP signaling pathway inhibitor, at least one growth factor from the FGF family, at least one SHH pathway inhibitor, and at least one RA signaling pathway activator) can be used. In some cases, the PKC activator comprises PdbU. In some cases, the PKC activator comprises TPB.

In some cases, Pdx1-positive pancreatic progenitor cells can be obtained by differentiating at least some primitive gut tube cells in a population into Pdx1-positive pancreatic progenitor cells, e.g., by contacting primitive gut tube cells with activin A, retinoic acid, KGF, Sant1, LDN193189, PdBU for a period of 2 days. In some cases, Pdx1-positive pancreatic progenitor cells can be obtained by differentiating at least some primitive gut tube cells in a population into Pdx1-positive pancreatic progenitor cells, e.g., by contacting primitive gut tube cells with DMH-1, activin A, retinoic acid, KGF, Sant1, LDN193189, PdBU for a first day, and activin A, retinoic acid, KGF, Sant1, LDN193189, PdBU for a second day. In some cases, Pdx1-positive pancreatic progenitor cells can be obtained by differentiating at least some primitive gut tube cells in a population into Pdx1-positive pancreatic progenitor cells, e.g., by contacting primitive gut tube cells with retinoic acid and KGF for a period of 2 days. In some cases, Pdx1-positive pancreatic progenitor cells can be obtained by differentiating at least some primitive gut tube cells in S3 medium.

Aspects of the disclosure involve NKX6.1-positive pancreatic progenitor cells. NKX6.1-positive pancreatic progenitor cells of use herein can be derived from any source or generated in accordance with any suitable protocol. In some aspects, Pdx1-positive pancreatic progenitor cells are differentiated to NKX6.1-positive pancreatic progenitor cells. In some aspects, the NKX6.1-positive pancreatic progenitor cells are further differentiated, e.g., to Ngn3-positive endocrine progenitor cells, or insulin-positive endocrine cells, followed by induction or maturation to SC-β cells.

In some aspects, a method of producing a NKX6.1-positive pancreatic progenitor cell from a Pdx 1-positive pancreatic progenitor cell comprises contacting a population of cells (e.g., under conditions that promote cell clustering) comprising Pdx1-positive pancreatic progenitor cells with at least two β cell-maturation factors comprising a) at least one growth factor from the fibroblast growth factor (FGF) family, b) a sonic hedgehog pathway inhibitor, and optionally c) a low concentration of a retinoic acid (RA) signaling pathway activator, to induce the differentiation of at least one Pdx1-positive pancreatic progenitor cell in the population into NKX6.1-positive pancreatic progenitor cells, wherein the NKX6.1-positive pancreatic progenitor cells expresses NKX6.1.

In some cases, the Pdx1-positive, NKX6.1-positive pancreatic progenitor cells are obtained by contacting Pdx1-positive pancreatic progenitor cells under conditions that promote cell clustering with i) at least one growth factor from the FGF family, ii) at least one SHH pathway inhibitor, and optionally iii) low concentrations of a RA signaling pathway activator, to induce the differentiation of at least some of the Pdx1-positive pancreatic progenitor cells into Pdx1-positive, NKX6.1-positive pancreatic progenitor cells, wherein the Pdx1-positive, NKX6.1-positive pancreatic progenitor cells expresses Pdx1 and NKX6.1. In some cases, the Pdx1-positive, NKX6.1-positive pancreatic progenitor cells are obtained by contacting Pdx1-positive pancreatic progenitor cells under conditions that promote cell clustering with i) at least one growth factor from the FGF family, ii) at least one SHH pathway inhibitor, and optionally iii) low concentrations of a RA signaling pathway activator, iv) ROCK inhibitor, and v) at least one growth factor from the TGF-β superfamily, to induce the differentiation of at least some of the Pdx1-positive pancreatic progenitor cells into Pdx1-positive, NKX6.1-positive pancreatic progenitor cells. In some cases, the Pdx1-positive, NKX6.1-positive pancreatic progenitor cells are obtained by contacting Pdx1-positive pancreatic progenitor cells under conditions that promote cell clustering with at least one growth factor from the FGF family.

In some cases, the Pdx1-positive pancreatic progenitor cells are produced from a population of pluripotent cells. In some cases, the Pdx1-positive pancreatic progenitor cells are produced from a population of iPS cells. In some cases, the Pdx1-positive pancreatic progenitor cells are produced from a population of ESC cells. In some cases, the Pdx1-positive pancreatic progenitor cells are produced from a population of definitive endoderm cells. In some cases, the Pdx1-positive pancreatic progenitor cells are produced from a population of primitive gut tube cells.

Any growth factor from the FGF family capable of inducing Pdx1-positive pancreatic-progenitor cells to differentiate into NKX6.1-positive pancreatic progenitor cells (e.g., alone, or with any combination of at least one SHH pathway inhibitor, or optionally at least one retinoic acid signaling pathway activator) can be used in the method provided herein. In some cases, the at least one growth factor from the FGF family comprises keratinocyte growth factor (KGF). In some cases, the at least one growth factor from the FGF family is selected from the group consisting of FGF2, FGF8B, FGF 10, and FGF21.

Any SHH pathway inhibitor capable of inducing Pdx1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, and at least one growth factor from the TGF-β superfamily) can be used in the method provided herein. In some cases, the SHH pathway inhibitor comprises Sant-1.

Any RA signaling pathway activator capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some cases, the RA signaling pathway activator comprises retinoic acid.

Any ROCK inhibitor capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some cases, the ROCK inhibitor comprises Thiazovivin, Y-27632, Fasudil/HA1077, or 14-1152.

Any activator from the TGF-β superfamily capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some cases, the activator from the TGF-β superfamily comprises Activin A or GDF8.

In some cases, the Pdx1-positive, NKX6.1-positive pancreatic progenitor cells are obtained by contacting Pdx1-positive pancreatic progenitor cells under conditions that promote cell clustering with KGF, Sant1, and RA, for a period of 5 or 6 days. In some cases, the Pdx1-positive, NKX6.1-positive pancreatic progenitor cells are obtained by contacting Pdx1-positive pancreatic progenitor cells under conditions that promote cell clustering with KGF, Sant1, RA, thiazovivin, and Activin A, for a period of 5 or 6 days. In some cases, the Pdx1-positive, NKX6.1-positive pancreatic progenitor cells are obtained by contacting Pdx1-positive pancreatic progenitor cells under conditions that promote cell clustering with KGF for a period of 5 or 6 days.

Aspects of the disclosure involve insulin-positive endocrine cells. Insulin-positive endocrine cells of use herein can be derived from any source or generated in accordance with any suitable protocol. In some aspects, NKX6.1-positive pancreatic progenitor cells are differentiated to insulin-positive endocrine cells, In some aspects, the insulin-positive endocrine cells are further differentiated, e.g., by induction or maturation to SC-β cells.

In some aspects, a method of producing an insulin-positive endocrine cell from an NKX6.1-positive pancreatic progenitor cell comprises contacting a population of cells (e.g., under conditions that promote cell clustering) comprising NKX6-1-positive pancreatic progenitor cells with a) a TGF-β signaling pathway inhibitor, b) a thyroid hormone signaling pathway activator, c) a protein kinase inhibitor, and/or a sonic hedgehog inhibitor, to induce the differentiation of at least one NKX6.1-positive pancreatic progenitor cell in the population into an insulin-positive endocrine cell, wherein the insulin-positive endocrine ceil expresses insulin. In some cases, insulin-positive endocrine cells express Pdx1, NKX6.1, NKX2.2, Mafb, glis3, Sur 1, Kir6.2, Znt8, SLC2A1, SLC2A3 and/or insulin.

Any TGF-β signaling pathway inhibitor capable of inducing the differentiation of NKX6.1-positive pancreatic progenitor cells to differentiate into insulin-positive endocrine cells (e.g., alone, or in combination with other β cell-maturation factors, e.g., a thyroid hormone signaling pathway activator) can be used. In some cases, the TGF-β signaling pathway comprises TGF-β receptor type I kinase signaling. In some cases, the TGF-β signaling pathway inhibitor comprises Alk5 inhibitor II. In some embodiments, a TGFβ-R1 kinase inhibitor (e.g., ALK5i) is present in the medium at a concentration of 1 μM-50 μM. In some embodiments, a TGFβ-R1 kinase inhibitor (e.g., ALK5i) is present in the medium at a concentration of 1 μM-50 μM, 1 μM-40 μM, 1 μM-30 μM, 1 μM-20 μM, 1 μM-10 μM, 10 μM-50 μM, 10 μM-40 μM, 10 μM-30 μM, 10 μM-20 μM, 20 μM-50 μM, 20 μM-40 μM, 20 μM-30 μM, 30 μM-50 μM, 30 μM-40 μM, or 40 μM-50 μM. In some embodiments, a TGFβ-R1 kinase inhibitor (e.g., ALK5i) is present in the medium at a concentration of 5 μM-20 μM (e.g., 5 μM, 10 μM, 15 μM, or 20 μM). In some embodiments, a TGFβ-R1 kinase inhibitor (e.g., ALK5i) is present in the medium at a concentration of 10 μM.

Any thyroid hormone signaling pathway activator capable of inducing the differentiation of NKX6.1-positive pancreatic progenitor cells to differentiate into insulin-positive endocrine cells (e.g., alone, or in combination with other β cell-maturation factors, e.g., a TGF-β signaling pathway inhibitor) can be used. In some cases, the thyroid hormone signaling pathway activator comprises triiodothyronine (T3) or GC-1. In some embodiments, a thyroid hormone (e.g., GC-1) is present in the medium at a concentration of 0.1 μM-10 μM. In some embodiments, a thyroid hormone (e.g., GC-1) is present in the medium at a concentration of 0.1 μM-10 μM, 0.1 μM-9 μM, 0.1 μM-8 μM, 0.1 μM-7 μM, 0.1 μM-6 μM, 0.1 μM-5 μM, 0.1 μM-4 μM, 0.1 μM-3 μM, 0.1 μM-2 μM, 0.1 μM-1 μM, 0.1 μM-0.5 μM, 0.5 μM-10 μM, 0.5 μM-9 μM, 0.5 μM-8 μM, 0.5 μM-7 μM, 0.5 μM-6 μM, 0.5 μM-5 μM, 0.5 μM-4 μM, 0.5 μM-3 μM, 0.5 μM-2 μM, 0.5 μM-1 μM, 1 μM-10 μM, 1 μM-9 μM, 1 μM-8 μM, 1 μM-7 μM, 1 μM-6 μM, 1 μM-5 μM, 1 μM-4 μM, 1 μM-3 μM, 1 μM-2 μM, 2 μM-10 μM, 2 μM-9 μM, 2 μM-8 μM, 2 μM-7 μM, 2 μM-6 μM, 2 μM-5 μM, 2 μM-4 μM, 2 μM-3 μM, 3 μM-10 μM, 3 μM-9 μM, 3 μM-8 μM, 3 μM-7 μM, 3 μM-6 μM, 3 μM-5 μM, 3 μM-4 μM, 4 μM-10 μM, 4 μM-9 μM, 4 μM-8 μM, 4 μM-7 μM, 4 μM-6 μM, 4 μM-5 μM, 5 μM-10 μM, 5 μM-9 μM, 5 μM-8 μM, 5 μM-7 μM, 5 μM-6 μM, 6 μM-10 μM, 6 μM-9 μM, 6 μM-8 μM, 6 μM-7 μM, 7 μM-10 μM, 7 μM-9 μM, 7 μM-8 μM, 8 μM-10 μM, 8 μM-9 μM, or 9 μM-10 μM. In some embodiments, a thyroid hormone (e.g., GC-1) is present in the medium at a concentration of 0.5 μM-5 μM (e.g., 0.5 μM, 1 μM, 1.5 μM, 2 μM, 2.5 μM, 3 μM, 3.5 μM, 4 μM, 4.5 μM, or 5 μM). In some embodiments, a thyroid hormone (e.g., GC-1) is present in the medium at a concentration of 1 μM.

In some cases, the method comprises contacting the population of cells (e.g., NKX6.1-positive pancreatic progenitor cells) with at least one additional factor. In some cases, the method comprises contacting the Pdx1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, and optionally v) a protein kinase inhibitor.

In some cases, the method comprises contacting the population of cells (e.g., NKX6.1-positive pancreatic progenitor cells) with at least one additional factor. In some cases, the method comprises contacting the Pdx1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) a ROCK inhibitor, vi) a histone methyltransferase EZH2 inhibitor (e.g., DZNEP) and vii) at least one bone morphogenic protein (BMP) signaling pathway inhibitor.

In some embodiments, any of the compositions comprise a ROCK inhibitor. In some embodiments, a Rho-associated, coiled-coil containing protein kinase (ROCK) inhibitor (e.g., thiazovivin) is present in the medium at a concentration of 1 μM-10 μM. In some embodiments, a Rho-associated, coiled-coil containing protein kinase (ROCK) inhibitor (e.g., thiazovivin) is present in the medium at a concentration of 1 μM-10 μM, 1 μM-9 μM, 1 μM-8 μM, 1 μM-7 μM, 1 μM-6 μM, 1 μM-5 μM, 1 μM-4 μM, 1 μM-3 μM, 1 μM-2 μM, 2 μM-10 μM, 2 μM-9 μM, 2 μM-8 μM, 2 μM-7 μM, 2 μM-6 μM, 2 μM-5 μM, 2 μM-4 μM, 2 μM-3 μM, 3 μM-10 μM, 3 μM-9 μM, 3 μM-8 μM, 3 μM-7 μM, 3 μM-6 μM, 3 μM-5 μM, 3 μM-4 μM, 4 μM-10 μM, 4 μM-9 μM, 4 μM-8 μM, 4 μM-7 μM, 4 μM-6 μM, 4 μM-5 μM, 5 μM-10 μM, 5 μM-9 μM, 5 μM-8 μM, 5 μM-7 μM, 5 μM-6 μM, 6 μM-10 μM, 6 μM-9 μM, 6 μM-8 μM, 6 μM-7 μM, 7 μM-10 μM, 7 μM-9 μM, 7 μM-8 μM, 8 μM-10 μM, 8 μM-9 μM, or 9 μM-10 μM. In some embodiments, a Rho-associated, coiled-coil containing protein kinase (ROCK) inhibitor (e.g., thiazovivin) is present in the medium at a concentration of 1 μM-5 μM (e.g., 1 μM, 1.5 μM, 2 μM, 2.5 μM, 3 μM, 3.5 μM, 4 μM, 4.5 μM, or 5 μM). In some embodiments, a Rho-associated, coiled-coil containing protein kinase (ROCK) inhibitor (e.g., thiazovivin) is present in the medium at a concentration of 2.5 μM.

Any γ-secretase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the γ-secretase inhibitor comprises XXI. In some cases, the γ-secretase inhibitor comprises DAPT. In some embodiments, a notch signaling pathway inhibitor (e.g., γ-secretase inhibitor such as XXI) is present in the medium at a concentration of 0.1 μM-10 μM, 0.1 μM-9 μM, 0.1 μM-8 μM, 0.1 μM-7 μM, 0.1 μM-6 μM, 0.1 μM-5 μM, 0.1 μM-4 μM, 0.1 μM-3 μM, 0.1 μM-2 μM, 0.1 μM-1 μM, 0.1 μM-0.5 μM, 0.5 μM-10 μM, 0.5 μM-9 μM, 0.5 μM-8 μM, 0.5 μM-7 μM, 0.5 μM-6 μM, 0.5 μM-5 μM, 0.5 μM-4 μM, 0.5 μM-3 μM, 0.5 μM-2 μM, 0.5 μM-1 μM, 1 μM-10 μM, 1 μM-9 μM, 1 μM-8 μM, 1 μM-7 μM, 1 μM-6 μM, 1 μM-5 μM, 1 μM-4 μM, 1 μM-3 μM, 1 μM-2 μM, 2 μM-10 μM, 2 μM-9 μM, 2 μM-8 μM, 2 μM-7 μM, 2 μM-6 μM, 2 μM-5 μM, 2 μM-4 μM, 2 μM-3 μM, 3 μM-10 μM, 3 μM-9 μM, 3 μM-8 μM, 3 μM-7 μM, 3 μM-6 μM, 3 μM-5 μM, 3 μM-4 μM, 4 μM-10 μM, 4 μM-9 μM, 4 μM-8 μM, 4 μM-7 μM, 4 μM-6 μM, 4 μM-5 μM, 5 μM-10 μM, 5 μM-9 μM, 5 μM-8 μM, 5 μM-7 μM, 5 μM-6 μM, 6 μM-10 μM, 6 μM-9 μM, 6 μM-8 μM, 6 μM-7 μM, 7 μM-10 μM, 7 μM-9 μM, 7 μM-8 μM, 8 μM-10 μM, 8 μM-9 μM, or 9 μM-10 μM. In some embodiments, a notch signaling pathway inhibitor (e.g., γ-secretase inhibitor such as XXI) is present in the medium at a concentration of 0.5 μM-5 μM (e.g., 0.5 μM, 1 μM, 1.5 μM, 2 μM, 2.5 μM, 3 μM, 3.5 μM, 4 μM, 4.5 μM, or 5 μM). In some embodiments, a notch signaling pathway inhibitor (e.g., 7-secretase inhibitor such as XXI) is present in the medium at a concentration of 2 μM.

Any growth factor from the EGF family capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some cases, the at least one growth factor from the EGF family comprises betacellulin. In some cases, at least one growth factor from the EGF family comprises EGF. In some embodiments, an epidermal growth factor (e.g., betacellulin) is present in the medium at a concentration of 10 ng/ml-50 ng/ml. In some embodiments, an epidermal growth factor (e.g., betacellulin) is present in the medium at a concentration of 10 ng/ml-50 ng/ml, 10 ng/ml-40 ng/ml, 10 ng/ml-30 ng/ml, 10 ng/ml-20 ng/ml, 20 ng/ml-50 ng/ml, 20 ng/ml-40 ng/ml, 20 ng/ml-30 ng/ml, 30 ng/ml-50 ng/ml, 30 ng/ml-40 ng/ml, or 40 ng/ml-50 ng/ml. In some embodiments, an epidermal growth factor (e.g., betacellulin) is present in the medium at a concentration of 10 ng/ml-30 ng/ml (e.g., 10 ng/ml, 20 ng/ml, 20 ng/ml). In some embodiments, an epidermal growth factor (e.g., betacellulin) is present in the medium at a concentration of 20 ng/ml.

Any RA signaling pathway activator capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the RA signaling pathway activator comprises RA. In some embodiments, retinoic acid is present in the medium at a concentration of 0.02 μM-0.5 μM. In some embodiments, retinoic acid is present in the medium at a concentration of 0.02 μM-0.5 μM, 0.05 μM-0.5 μM, 0.1 μM-0.5 μM, 0.15 μM-0.5 μM, 0.2 μM-0.5 μM, 0.25 μM-0.5 μM, 0.3 μM-0.5 μM, 0.35 μM-0.5 μM, 0.4 μM-0.5 μM, 0.45 μM-0.5 μM, 0.02 μM-0.4 μM, 0.05 μM-0.4 μM, 0.1 μM-0.4 μM, 0.15 μM-0.4 μM, 0.2 μM-0.4 μM, 0.25 μM-0.4 μM, 0.3 μM-0.4 μM, 0.35 μM-0.4 μM, 0.02 μM-0.3 μM, 0.05 μM-0.3 μM, 0.1 μM-0.3 μM, 0.15 μM-0.3 μM, 0.2 μM-0.3 μM, 0.25 μM-0.3 μM, 0.02 μM-0.2 μM, 0.05 μM-0.2 μM, 0.1 μM-0.2 μM, 0.15 μM-0.2 μM, 0.02 μM-0.1 μM, 0.05 μM-0.1 μM, or 0.02 μM-0.05 μM. In some embodiments, retinoic acid is present in the medium at a concentration of 0.02 μM-0.2 μM (e.g., 0.02 μM, 0.05 μM, 0.1 μM, 0.15 μM, or 0.2 μM). In some embodiments, retinoic acid is present in the medium at a concentration of 0.05 μM.

Any SHH pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used in the method provided herein. In some cases, the SHH pathway inhibitor comprises Sant1. In some embodiments, a sonic hedgehog (SHH) signaling pathway inhibitor (e.g., SANT-1) is present in the medium at a concentration of 0.1 μM-10 μM, 0.1 μM-9 μM, 0.1 μM-8 μM, 0.1 μM-7 μM, 0.1 μM-6 μM, 0.1 μM-5 μM, 0.1 μM-4 μM, 0.1 μM-3 μM, 0.1 μM-2 μM, 0.1 μM-1 μM, 0.1 μM-0.5 μM, 0.5 μM-10 μM, 0.5 μM-9 μM, 0.5 μM-8 μM, 0.5 μM-7 μM, 0.5 μM-6 μM, 0.5 μM-5 μM, 0.5 μM-4 μM, 0.5 μM-3 μM, 0.5 μM-2 μM, 0.5 μM-1 μM, 1 μM-10 μM, 1 μM-9 μM, 1 μM-8 μM, 1 μM-7 μM, 1 μM-6 μM, 1 μM-5 μM, 1 μM-4 μM, 1 μM-3 μM, 1 μM-2 μM, 2 μM-10 μM, 2 μM-9 μM, 2 μM-8 μM, 2 μM-7 μM, 2 μM-6 μM, 2 μM-5 μM, 2 μM-4 μM, 2 μM-3 μM, 3 μM-10 μM, 3 μM-9 μM, 3 μM-8 μM, 3 μM-7 μM, 3 μM-6 μM, 3 μM-5 μM, 3 μM-4 μM, 4 μM-10 μM, 4 μM-9 μM, 4 μM-8 μM, 4 μM-7 μM, 4 μM-6 μM, 4 μM-5 μM, 5 μM-10 μM, 5 μM-9 μM, 5 μM-8 μM, 5 μM-7 μM, 5 μM-6 μM, 6 μM-10 μM, 6 μM-9 μM, 6 μM-8 μM, 6 μM-7 μM, 7 μM-10 μM, 7 μM-9 μM, 7 μM-8 μM, 8 μM-10 μM, 8 μM-9 μM, or 9 μM-10 μM. In some embodiments, a sonic hedgehog (SHH) signaling pathway inhibitor (e.g., SANT-1) is present in the medium at a concentration of 0.1 μM-0.5 μM (e.g., 0.1 μM, 0.15 μM, 0.2 μM, 0.25 μM, 0.3 μM, 0.35 μM, 0.4 μM, 0.45 μM, or 0.5 μM). In some embodiments, a sonic hedgehog (SHH) signaling pathway inhibitor (e.g., SANT-1) is present in the medium at a concentration of 0.25 μM.

Any BMP signaling pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the BMP signaling pathway inhibitor comprises LDN193189. In some embodiments, a bone morphogenetic (BMP) signaling pathway inhibitor (e.g., LDN-193189) is present in the medium at a concentration of 0.05 μM-0.5 μM. In some embodiments, a bone morphogenetic (BMP) signaling pathway inhibitor (e.g., LDN-193189) is present in the medium at a concentration of 0.05 μM-0.5 μM, 0.1 μM-0.5 μM, 0.15 μM-0.5 μM, 0.2 μM-0.5 μM, 0.25 μM-0.5 μM, 0.3 μM-0.5 μM, 0.35 μM-0.5 μM, 0.4 μM-0.5 μM, 0.45 μM-0.5 μM, 0.05 μM-0.4 μM, 0.1 μM-0.4 μM, 0.15 μM-0.4 μM, 0.2 μM-0.4 μM, 0.25 μM-0.4 μM, 0.3 μM-0.4 μM, 0.35 μM-0.4 μM, 0.05 μM-0.3 μM, 0.1 μM-0.3 μM, 0.15 μM-0.3 μM, 0.2 μM-0.3 μM, 0.25 μM-0.3 μM, 0.05 μM-0.2 μM, 0.1 μM-0.2 μM, 0.15 μM-0.2 μM, or 0.05 μM-0.1 μM. In some embodiments, a bone morphogenetic (BMP) signaling pathway inhibitor (e.g., LDN-193189) is present in the medium at a concentration of 0.05 μM-0.2 μM (e.g., 0.05 μM, 0.1 μM, 0.15 μM, or 0.2 μM). In some embodiments, a bone morphogenetic (BMP) signaling pathway inhibitor (e.g., LDN-193189) is present in the medium at a concentration of 0.1 μM.

In some cases, the population of cells is optionally contacted with a protein kinase inhibitor. In some cases, the population of cells is not contacted with the protein kinase inhibitor. In some cases, the population of cells is contacted with the protein kinase inhibitor. Any protein kinase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the protein kinase inhibitor comprises staurosporine. In some embodiments, a protein kinase inhibitor (e.g., staurosporine) is present in the medium at a concentration of 0.5 nM-10 nM. In some embodiments, a protein kinase inhibitor (e.g., staurosporine) is present in the medium at a concentration of 0.5 nM-10 nM, 0.5 nM-9 nM, 0.5 nM-8 nM, 0.5 nM-7 nM, 0.5 nM-6 nM, 0.5 nM-5 nM, 0.5 nM-4 nM, 0.5 nM-3 nM, 0.5 nM-2 nM, 0.5 nM-1 nM, 1 nM-10 nM, 1 nM-9 nM, 1 nM-8 nM, 1 nM-7 nM, 1 nM-6 nM, 1 nM-5 nM, 1 nM-4 nM, 1 nM-3 nM, 1 nM-2 nM, 2 nM-10 nM, 2 nM-9 nM, 2 nM-8 nM, 2 nM-7 nM, 2 nM-6 nM, 2 nM-5 nM, 2 nM-4 nM, 2 nM-3 nM, 3 nM-10 nM, 3 nM-9 nM, 3 nM-8 nM, 3 nM-7 nM, 3 nM-6 nM, 3 nM-5 nM, 3 nM-4 nM, 4 nM-10 nM, 4 nM-9 nM, 4 nM-8 nM, 4 nM-7 nM, 4 nM-6 nM, 4 nM-5 nM, 5 nM-10 nM, 5 nM-9 nM, 5 nM-8 nM, 5 nM-7 nM, 5 nM-6 nM, 6 nM-10 nM, 6 nM-9 nM, 6 nM-8 nM, 6 nM-7 nM, 7 nM-10 nM, 7 nM-9 nM, 7 nM-8 nM, 8 nM-10 nM, 8 nM-9 nM, or 9 nM-10 nM. In some embodiments, a protein kinase inhibitor (e.g., staurosporine) is present in the medium at a concentration of 1 nM-5 nM (e.g., 1 nM, 2 nM, 3 nM, 4 nM, or 5 nM). In some embodiments, a protein kinase inhibitor (e.g., staurosporine) is present in the medium at a concentration of 3 nM.

In some embodiments, any of the compositions disclosed herein comprises a histone methyltransferase EZH2 inhibitor (e.g., DZNEP). In some embodiments, a histone methyltransferase EZH2 inhibitor (e.g., DZNEP) is present in the medium at a concentration of 0.05 μM-0.5 μM. In some embodiments, a histone methyltransferase EZH2 inhibitor (e.g., DZNEP) is present in the medium at a concentration of 0.05 μM-0.5 μM, 0.1 μM-0.5 μM, 0.15 μM-0.5 μM, 0.2 μM-0.5 μM, 0.25 μM-0.5 μM, 0.3 μM-0.5 μM, 0.35 μM-0.5 μM, 0.4 μM-0.5 μM, 0.45 μM-0.5 μM, 0.05 μM-0.4 μM, 0.1 μM-0.4 μM, 0.15 μM-0.4 μM, 0.2 μM-0.4 μM, 0.25 μM-0.4 μM, 0.3 μM-0.4 μM, 0.35 μM-0.4 μM, 0.05 μM-0.3 μM, 0.1 μM-0.3 μM, 0.15 μM-0.3 μM, 0.2 μM-0.3 μM, 0.25 μM-0.3 μM, 0.05 μM-0.2 μM, 0.1 μM-0.2 μM, 0.15 μM-0.2 μM, or 0.05 μM-0.1 μM. In some embodiments, a histone methyltransferase EZH2 inhibitor (e.g., DZNEP) is present in the medium at a concentration of 0.05 μM-0.2 μM (e.g., 0.05 μM, 0.1 μM, 0.15 μM, or 0.2 μM). In some embodiments, a histone methyltransferase EZH2 inhibitor (e.g., DZNEP) is present in the medium at a concentration of 0.1 μM.

In some cases, the method comprises contacting the population of cells (e.g., NKX6.1-positive pancreatic progenitor cells) with XXI, Alk5i, T3, RA, Sant1, and betacellulin for a period of 7 days, to induce the differentiation of at least one NKX6.1-positive pancreatic progenitor cell in the population into an insulin-positive endocrine cell, wherein the insulin-positive endocrine cell expresses insulin. In some cases, the method comprises contacting the population of cells (e.g., NKX6.1-positive pancreatic progenitor cells) with XXI, Alk5i, T3, RA, Sant1, betacellulin, and LDN193189 for a period of 7 days, to induce the differentiation of at least one NKX6.1-positive pancreatic progenitor cell in the population into an insulin-positive endocrine cell, wherein the insulin-positive endocrine ceil expresses insulin.

In some cases, the method comprises culturing the population of cells (e.g., NKX6.1-positive pancreatic progenitor cells) in a BE5 medium, to induce the differentiation of at least one NKX6.1-positive pancreatic progenitor cell in the population into an insulin-positive endocrine cell, wherein the insulin-positive endocrine cell expresses insulin.

Aspects of the disclosure involve generating non-native pancreatic β cells which resemble endogenous mature β cells in form and function, but nevertheless are distinct from native β cells.

In some cases, the insulin-positive endocrine cells generated using the method provided herein can form a cell cluster, alone or together with other types of cells, e.g., precursors thereof, e.g., stem cell, definitive endoderm cells, primitive gut tube cell, Pdx1-positive pancreatic progenitor cells, or NKX6.1-positive pancreatic progenitor cells. In some cases, the cell cluster comprising the insulin-positive endocrine cells can be reaggregated using the method provided herein. The reaggregation of the cell cluster can enrich the insulin-positive endocrine cells. In some cases, the insulin-positive endocrine cells in the cell cluster can be further matured into pancreatic β cells. For example, after reaggregation, the second cell cluster can exhibit in vitro GSIS, resembling native pancreatic islet. For example, after reaggregation, the second cell cluster can comprise non-native pancreatic β cell that exhibits in vitro GSIS.

Stage 6 cells as provided herein may or may not be subject to the dissociation and reaggregation process as described herein. In some cases, the cell population comprising the insulin-positive endocrine cells can be directly induced to mature into Sc-β cells. In some cases, the maturation factors can comprise at least one inhibitor of TGF-β signaling pathway and thyroid hormone signaling pathway activator as described herein. In some cases, Sc-β cells can be obtained by contacting a population of cells comprising insulin-positive endocrine cells with Alk5i and T3. In some cases, the insulin-positive endocrine cells can be matured in a CMRLs medium supplemented with 10% FBS. In other cases, Sc-β cells can be obtained by culturing the population of cells containing the insulin-positive endocrine cells in a MCDB131 medium that can be supplemented by 2% BSA. In some cases, the MCDB131 medium with 2% BSA for maturation of insulin-positive endocrine cells into Sc-β cells can comprise no small molecule factors as described herein. In some case, the MCDB131 medium with 2% BSA for maturation of insulin-positive endocrine cells into Sc-β cells can comprise no serum (e.g., no FBS).

One aspect of the present disclosure provides a method of cryopreservation. As provided herein, the cell population comprising non-native pancreatic β cells can be stored via cryopreservation. For instances, the cell population comprising non-native β cells, e.g., Stage 6 cells in some cases, can be dissociated into cell suspension, e.g., single cell suspension, and the cell suspension can be cryopreserved, e.g., frozen in a cryopreservation solution. The dissociation of the cells can be conducted by any of the technique provided herein, for example, by enzymatic treatment. The cells can be frozen at a temperature of at highest −20° C., at highest −30° C., at highest −40° C., at highest −50° C., at highest −60° C., at highest −70° C., at highest −80° C., at highest −90° C., at highest −100° C., at highest −110° C., at highest −120° C., at highest −130° C., at highest −140° C., at highest −150° C., at highest −160° C., at highest −170° C., at highest −180° C., at highest −190° C., or at highest −200° C. In some cases, the cells are frozen at a temperature of about −80° C. In some cases, the cells are frozen at a temperature of about −195° C. Any cooling methods can be used for providing the low temperature needed for cryopreservation, such as, but not limited to, electric freezer, solid carbon dioxide, and liquid nitrogen. In some cases, any cryopreservation solution available to one skilled in the art can be used for incubating the cells for storage at low temperature, including both custom made and commercial solutions. For example, a solution containing a cryoprotectant can be used. The cryoprotectant can be an agent that is configured to protect the cell from freezing damage. For instance, a cryoprotectant can be a substance that can lower the glass transition temperature of the cryopreservation solution. Exemplary cryoprotectants that can be used include DMSO (dimethyl sulfoxide), glycols (e.g., ethylene glycol, propylene glycol and glycerol), dextran (e.g., dextran-40), and trehalose. Additional agents can be added in to the cryopreservation solution for other effects. In some cases, commercially available cryopreservation solutions can be used in the method provided herein, for instance, FrostaLife™, pZerve™, Prime-XV®, Gibco Synth-a-Freeze Cryopreservation Medium, STEM-CELLBANKER®, CryoStor® Freezing Media, HypoThermosol® FRS Preservation Media, and CryoDefend® Stem Cells Media.

In some cases, a cell cluster can be cryopreserved before being subject to reaggregation using the method provided herein. In some cases, a cell cluster can be dissociated into cell suspension as provided herein and then cryopreserved. After cryopreservation for a certain period of time, the cryopreserved cells can be thawed and cultured for reaggregation using the method as provided herein. Cryopreservation as provided herein can prolong the availability of the pancreatic β cells or their precursors. In some cases, during differentiation of non-native pancreatic β cells from precursors thereof or stem cells, the intermediate cell population can be preserved following the method provided herein until the non-native pancreatic β cells are desired, e.g., for transplanting into a human patient. In some cases, the cells can be cryopreserved for any desired period of time before their further use or further processing of the cells, e.g., reaggregation. For example, the cells can be cryopreserved for at least 1 day, at least 5 days, at least 10 days, at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, at least 5 years, or at least 10 years.

In some embodiments, compositions and methods disclosed herein improve cell recovery or yield after they are cryopreserved and subsequently thawed. Increased cell recovery can comprise an increased number or percentage of viable cells. Increased cell recovery can comprise an increased number or percentage of a population of interest (e.g., ISL1-positive and NKX6.1 positive cells). In some embodiments, increased cell recovery is achieved after contacting the cells with a combination of agents disclosed herein. For example, in some embodiments, contacting cells with any one or more of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, and/or carnitine as disclosed herein increases cell recovery. In some embodiments, cell recovery is increased when the cells are contacted with the agents prior to cryopreservation. In some embodiments, cell recovery is increased when the cells are contacted with the agents after cryopreservation.

Sc-β cells can exhibit a response to at least one glucose challenge. In some cases, the SC-β cells exhibit a response to at least two sequential glucose challenges. In some cases, the SC-β cells exhibit a response to at least three sequential glucose challenges. In some cases, the SC-β cell exhibits a response to multiple (e.g., sequential) glucose challenges that resembles the response of endogenous human islets to multiple glucose challenges, In some cases, the SC-β cells are capable of releasing or secreting insulin in response to two consecutive glucose challenges. In some cases, the SC-β cells are capable of releasing or secreting insulin in response to three consecutive glucose challenges. In some cases, the SC-β cells are capable of releasing or secreting insulin in response to four consecutive glucose challenges. In some cases, the SC-β cells are capable of releasing or secreting insulin in response to five consecutive glucose challenges. In some cases, the SC-β cells release or secrete insulin in response to perpetual consecutive glucose challenges. In some cases, cells can be assayed to determine whether they respond to sequential glucose challenges by determining whether they repeatedly increase intracellular Ca², as described in the examples herein.

In some cases, a method as provided herein can start with a cell population comprising NKX6.1-positive pancreatic progenitor cells. NKX6.1-positive cells can be differentiated into NKX6.1-positive and C-peptide-positive endocrine cells by contacting the NKX6.1-positive cells with at least one factor from EGF superfamily, e.g., betacellulin. In some cases, NKX6.1-positive and C-peptide-positive endocrine cells can also be referred to as insulin-positive endocrine cells. In some cases, one characteristic of insulin-positive endocrine cells can be expression of chromogranin A. In some cases, the population comprising insulin-endocrine cells can be dissociated and reaggregated into a cell cluster as described above.

In some cases, conditions that promote cell clustering comprise a suspension culture. In some cases, the period of time comprises a period of time sufficient to maximize the number of cells co-expressing C-peptide and Nkx6-1. In some cases, the period of time is at least 5 days. In some cases, the period of time is between 5 days and 7 days. In some cases, the period of time is at least 7 days. In some cases, the suspension culture is replenished every day (e.g., with β cell-maturation factors). In some cases, a period of time of between 5 days and 7 days maximizes the number of cells co-expressing C-peptide and NKX6.1.

In some cases, at least 15% of the NKX6.1-positive pancreatic progenitor cells in the population are induced to differentiate into insulin-positive endocrine cells. In some cases, at least 99% of the NKX6.1-positive pancreatic progenitor cells in the population are induced to differentiate into insulin-positive endocrine cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into Pdx1-positive pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor, v) a PKC activator, and optionally vi) a ROCK inhibitor, for a period of 2 days; d) differentiating at least some of the Pdx1-positive pancreatic progenitor cells into Pdx1-positive, NKX6.1-positive pancreatic progenitor cells by a process of contacting the Pdx1-positive pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, every other day for a period of 5 days, wherein the NKX6.1-positive pancreatic progenitor cells expresses Pdx 1 and NKX6.1; e) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, and vi) at least one growth factor from the epidermal growth factor (EGF) family, every other day for a period of between five and seven days, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and f) differentiating at least some of the Pdx1-positive, NKX6.1-positive, insulin-positive endocrine cells into SC-β cells by a process of contacting the Pdx1-positive, NKX6.1-positive, insulin-positive endocrine cells under conditions that promote cell clustering with i) a transforming growth factor β (TGF-β) signaling pathway inhibitor, ii) a thyroid hormone signaling pathway activator, and optionally iii) a protein kinase inhibitor, every other day for a period of between 7 and 14 days to induce the in vitro maturation of at least some of the Pdx1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into Pdx1-positive pancreatic progenitor cells by a process of contacting the primitive gut tube cells with i) retinoic acid signaling pathway activator and ii) at least one factor from the FGF family for a period of 2 days; d) differentiating at least some of the Pdx1-positive pancreatic progenitor cells into Pdx1-positive, NKX6.1-positive pancreatic progenitor cells by a process of contacting the Pdx1-positive pancreatic progenitor cells under conditions that promote cell clustering with at least one growth factor from the FGF family every other day for a period of 5 days, wherein the NKX6.1-positive pancreatic progenitor cells expresses Pdx1 and NKX6.1; e) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, vi) at least one growth factor from the epidermal growth factor (EGF) family, and vii) BMP signaling pathway inhibitor, every other day for a period of between five and seven days, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and f) differentiating at least some of the Pdx1-positive, NKX6.1-positive, insulin-positive endocrine cells into SC-β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into SC-β cells, wherein the SC-β cells exhibit a GS1S response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into Pdx1-positive, NKX6.1-positive pancreatic progenitor cells under suitable conditions; b) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, vi) at least one growth factor from the epidermal growth factor (EGF) family, and vii) BMP signaling pathway inhibitor, every other day for a period of between five and seven days, wherein the Pdx1-positive, NKX6.1, insulin-positive endocrine cells express Pdx1, NKX6.1, NKX2.2, Mafb, glis3, Sur1, Kir6.2, Znt8, SLC2A1, SLC2A3 and/or insulin; and c) differentiating at least some of the Pdx1-positive, NKX6.1-positive, insulin-positive endocrine cells into SC-β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating a cell cluster containing pancreatic β cells, the method comprising: a) obtaining a cell population comprising NKX6.1-positive pancreatic progenitor cells; b) differentiating at least some of the NKX6.1-positive pancreatic progenitor cells into NKX6.1-positive, insulin-positive (or C-peptide-positive) endocrine cells by a process of contacting the NKX6.1-positive pancreatic progenitor cells with at least one growth factor from the epidermal growth factor (EGF) family, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and c) differentiating at least some of the Pdx 1-positive, NKX6.1-positive, insulin-positive endocrine cells into pancreatic β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into pancreatic β cells, wherein the pancreatic β cells exhibit a GSIS response in vitro and/or in vivo. In some cases, the GSIS response resembles the GSIS response of an endogenous mature β cells.

In some embodiments, the disclosure provides compositions comprising a population of β cells that have been contacted in vitro with at least one agent selected from the group consisting of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, and carnitine, wherein said population of β cells exhibit increased glucose stimulated insulin secretion compared to a corresponding population of β cells that have not been contacted with said at least one agent. In some embodiments, said population of cells that have been contacted with at least two, three, four, five, six, or seven of the agents selected from the group consisting of glutamate, acetate, β-hydroxybutyrate, L-carnitine, taurine, formate, and biotin. In some embodiments, the population of cells are further contacted with zinc (e.g., ZnSO₄).

In some embodiments, the disclosure provides compositions comprising a population of β cells that have been contacted in vitro with at least one agent selected from the group consisting of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), and glutamine, wherein said population of β cells exhibit increased glucose stimulated insulin secretion compared to a corresponding population of β cells that have not been contacted with said at least one agent. In some embodiments, said population of cells that have been contacted with at least two, three, four, five, or six of the agents selected from the group consisting of formate, acetate, biotin, β-Hydroxybutyrate, taurine, and glutamine.

In some aspects, the disclosure provides methods comprising: (a) obtaining a first population of cells comprising a plurality of cell clusters comprising insulin-positive cells; (b) dissociating at least a portion of the plurality of cell clusters in the first population of cells in vitro; (c) contacting the first population of cells comprising at least a portion of the dissociated cell clusters with a first composition in vitro to obtain a second population of cells comprising a plurality of cells clusters comprising a plurality of insulin-positive cells, and (d) contacting the second population of insulin-positive cells in vitro with a second composition, wherein the second composition is different from the first composition, thereby differentiating at least a portion of said second population of insulin-positive cells into a third population of cells comprising a plurality of β cells, wherein the third population of cells comprises a higher percentage of viable β cells as compared to a corresponding population of β cells comprising β cells derived from the first population of cells which is not contacted with the first composition.

In some embodiments, the second population of cells comprises at least about 10%, 20%, 30%, 40%, 50%, 60%, 70%, 80%, or 90% more viable insulin-positive endocrine cells after from about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days of contacting the first cell population with the first composition as compared to a corresponding population of cells comprising insulin-positive endocrine cells which is not contacted with the first composition.

In some embodiments, the third population of cells comprises at least about 10%, 20%, 30%, 40%, 50%, 60%, 70%, 80%, or 90% more viable β cells as compared to a corresponding population of cells comprising β cells derived from the first population of cells which is not contacted with the first composition. In some embodiments, the third population of cells comprises at least about 10%, 20%, 30%, 40%, 50%, 60%, 70%, 80%, or 90% more viable β cells after about 1, 2, 3, 4, 5, 6, 7, 8, 9, or 10 days of contacting the first cell population with the first composition as compared to a corresponding population of cells comprising β cells derived from the first population of cells which is not contacted with the first composition.

In some embodiments, the first composition compromises two, three, four, or five of the agents disclosed herein. In some embodiments, the first composition compromises three, four, five, six, or seven of the agents disclosed herein.

In some embodiments, the first composition or the second composition comprises at least one agent selected from the group consisting of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), and glutamine.

In some embodiments, the first composition or the second composition comprises at least one agent selected from the group consisting of a one carbon metabolism pathway intermediate (e.g., formate), an acetyl CoA-related metabolite (e.g., acetate), a vitamin (e.g., biotin), an HDAC inhibitor (e.g., β-Hydroxybutyrate), a redox homeostasis regulator (e.g., taurine), glutamine, glutamate, and carnitine.

In some embodiments, the first composition or the second composition comprises a one carbon metabolism pathway intermediate. In some embodiments the one carbon metabolism pathway intermediate is formate.

In some embodiments, the first composition or the second composition comprises an acetyl CoA-related metabolite. In some embodiments the acetyl CoA-related metabolite is acetate.

In some embodiments, the first composition or the second composition comprises an HDAC inhibitor. In some embodiments the HDAC inhibitor is β-Hydroxybutyrate.

In some embodiments, the first composition or the second composition comprises a redox homeostasis regulator. In some embodiments the redox homeostasis regulator is taurine.

In some embodiments, the first composition or the second composition comprises glutamine.

In some embodiments, the first composition or the second composition comprises glutamate.

In some embodiments, the first composition or the second composition comprises carnitine.

In some embodiments, the first composition or the second composition comprises at least one vitamin. In some embodiments, the at least one vitamin is biotin or riboflavin.

In some embodiments, the first composition comprises at least one of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor.

In some embodiments, the first composition or the second composition does not comprise one or more of a MGLL inhibitor, a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, or a protein kinase inhibitor. In some embodiments, the first composition or the second composition does not comprise a MGLL inhibitor. In some embodiments, the first composition or the second composition does not comprise a TGF-β signaling pathway inhibitor. In some embodiments, the first composition or the second composition does not comprise a thyroid hormone signaling pathway activator. In some embodiments, the second composition does not comprise a bone morphogenic protein (BMP) type 1 receptor inhibitor. In some embodiments, the first composition or the second composition does not comprise a Rho-associated coiled-coil containing protein kinase (ROCK) inhibitor. In some embodiments, the first composition or the second composition does not comprise a histone methyltransferase inhibitor. In some embodiments, the first composition or the second composition does not comprise a protein kinase inhibitor. In some embodiments, the first composition or the second composition does not comprise a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the first composition and/or the second composition comprises at least one amino acid. In some embodiments, the at least one amino acid is alanine, glutamate, glutamine, glycine, proline, threonine, or tryptophan. In some embodiments, the at least one amino acid is arginine, histidine, lysine, aspartic acid, glutamic acid, serine, asparagine, glutamine, cysteine, selenocysteine, alanine, valine, isoleucine, leucine, methionine, phenylalanine, tyrosine, glutamate, glycine, proline, threonine, or tryptophan.

In some embodiments, methods of the disclosure are in the absence of a selection step. For example, viable cells, a percentage of a cell population disclosed herein, the diameter of cell clusters, and other parameters disclosed herein can be evaluated without a selection step.

### Methods of Enriching Stem Cell Derived Beta Cells

Provided herein are methods of isolating or enriching for a population of 3 cells (e.g., stem cell derived β cells) from a heterogeneous population of cells, e.g., a mixed population of cells comprising β cells (e.g., stem cell derived β cells) or precursors thereof from which the R cells (e.g., stem cell derived β cells) cells were derived. A population of 3 cells (e.g., stem cell derived β cells) produced by any of the above-described processes can be enriched, isolated and/or purified by using a cell surface marker (e.g., CD49a, CD29, CD99, CD10, CD59, CD141, CD165, G46-2.6, CD44, CD57) present on the β cells (e.g., stem cell derived β cells), which is not present on the insulin-positive endocrine cell or precursor thereof from which it was derived, enterochromaffin cells (EC cells), and/or α-cells (e.g., stem cell derived a cells). Such cell surface markers are also referred to as an affinity tag which is specific for a β cell (e.g., stem cell derived β cell). In some embodiments, the cell surface marker is an inducible cell surface marker. For example, CD49a can be induced to the surface by certain signals.

In some embodiments, differentiated β cells (e.g., stem cell derived β cells) can be sorted and enriched from other cells, including a cells and endocrine cells. In some embodiments, differentiated β cells (e.g., stem cell derived β cells) can be sorted and enriched from other cells, including a cells, EC cells, and immature insulin-positive endocrine cells, by contacting the population of cells with an agent that binds CD49a. In some embodiments, the agent is an antibody or antigen binding fragment thereof that binds CD49a expressed on the surface of differentiated β cells (e.g., stem cell derived β cells).

In some embodiments, the agent is a ligand or other binding agent that specifically binds CD49a that is present on the cell surface of a differentiated β cells (e.g., stem cell derived β cells) In some embodiments, an antibody which binds to CD49a present on the surface of a SC-β cell (e.g. a human SC-β cell) is used as an affinity tag for the enrichment, isolation or purification of chemically induced (e.g. by contacting with at least one β cell maturation factor as described herein) SC-β cells produced by the methods described herein. Such antibodies are known and commercially available.

The skilled artisan will readily appreciate the processes for using antibodies for the enrichment, isolation and/or purification of SC-β cell. For example, in some embodiments, the reagent, such as an antibody, is incubated with a cell population comprising SC-β cells, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FACS-conjugated antibody that is capable of binding to the primary antibody. The SC-β cells are then washed, centrifuged, and resuspended in buffer. The SC-β cell suspension is then analyzed and sorted using a fluorescence activated cell sorter (FACS). Antibody-bound, fluorescent reprogrammed cells are collected separately from non-bound, non-fluorescent cells (e.g. immature insulin-producing cells, a cells, and endocrine cells), thereby resulting in the isolation of SC-β cells from other cells present in the cell suspension, e.g. insulin-positive endocrine cells or precursors thereof, or immature, insulin-producing cell (e.g. other differentiated cell types).

In some embodiments, the isolated cell composition that comprises differentiated β cells (e.g., stem cell derived β cells) can be further purified by using an alternate affinity-based method or by additional rounds of sorting using the same or different markers that are specific for differentiated β cells (e.g., stem cell derived β cells). For example, in some embodiments, FACS sorting is used to first isolate a SC-β cell which expresses NKX6-1, either alone or with the expression of C-peptide, or alternatively with a β cell marker disclosed herein from cells that do not express one of those markers (e.g. negative cells) in the cell population. A second FACS sorting, e.g. sorting the positive cells again using FACS to isolate cells that are positive for a different marker than the first sort enriches the cell population for reprogrammed cells. In an alternative embodiment, FACS sorting is used to separate cells by negatively sorting for a marker that is present on most insulin-positive endocrine cells or precursors thereof but is not present on SC-β cells.

In some embodiments, differentiated β cells (e.g., stem cell derived β cells) are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above. For example, in some embodiments, at least one copy of a nucleic acid encoding GFP or a biologically active fragment thereof is introduced into at least one insulin-positive endocrine cell which is first chemically induced into a SC-β cell, where a downstream of a promoter expressed in SC-β cell, such as the insulin promoter, such that the expression of the GFP gene product or biologically active fragment thereof is under control of the insulin promoter.

In addition to the procedures just described, chemically induced SC-β cells may also be isolated by other techniques for cell isolation. Additionally, SC-β cells may also be enriched or isolated by methods of serial subculture in growth conditions which promote the selective survival or selective expansion of the SC-β cell. Such methods are known by persons of ordinary skill in the art, and may include the use of agents such as, for example, insulin, members of the TGF-beta family, including Activin A, TGF-beta 1, 2, and 3, bone morphogenic proteins (BMP-2, -3, -4, -5, -6, -7, -11, -12, and -13), fibroblast growth factors-1 and -2, platelet-derived growth factor-AA, and —BB, platelet rich plasma, insulin-like growth factors (IGF-I, II) growth differentiation factor (GDF-5, -6, -7, -8, -10, -11, -15), vascular endothelial cell-derived growth factor (VEGF), Hepatocyte growth factor (HGF), pleiotrophin, endothelin, Epidermal growth factor (EGF), beta-cellulin, among others. Other pharmaceutical compounds can include, for example, nicotinamide, glucagon like peptide-1 (GLP-1) and II, GLP-1 and 2 mimetibody, Exendin-4, retinoic acid, parathyroid hormone.

Using the methods described herein, enriched, isolated and/or purified populations of differentiated β cells (e.g., stem cell derived β cells) can be produced in vitro from insulin-positive endocrine cells or precursors thereof (which were differentiated from pluripotent stem cells by the methods described herein). In some embodiments, preferred enrichment, isolation and/or purification methods relate to the in vitro production of human differentiated β cells (e.g., stem cell derived β cells) from human insulin-positive endocrine cells or precursors thereof, which were differentiated from human pluripotent stem cells, or from human induced pluripotent stem (iPS) cells. In such an embodiment, where SC-β cells are differentiated from insulin-positive endocrine cells, which were previously derived from definitive endoderm cells, which were previously derived from iPS cells, the SC-β cell can be autologous to the subject from whom the cells were obtained to generate the iPS cells.

Using the methods described herein, isolated cell populations of differentiated β cells (e.g., stem cell derived β cells) are enriched in differentiated β cell (e.g., stem cell derived β cell) content by at least about 2- to about 1000-fold as compared to a population of cells before the chemical induction of the insulin-positive endocrine cell or precursor population. In some embodiments, differentiated β cells (e.g., stem cell derived β cells) can be enriched by at least about 5- to about 500-fold as compared to a population before the chemical induction of an insulin-positive endocrine cell or precursor population. In other embodiments, differentiated β cells (e.g., stem cell derived β cells) cells can be enriched from at least about 10- to about 200-fold as compared to a population before the chemical induction of insulin-positive endocrine cell or precursor population. In still other embodiments, differentiated β cells (e.g., stem cell derived β cells) cell can be enriched from at least about 20- to about 100-fold as compared to a population before the chemical induction of insulin-positive endocrine cell or precursor population. In yet other embodiments, differentiated β cells (e.g., stem cell derived β cells) can be enriched from at least about 40- to about 80-fold as compared to a population before the chemical induction of insulin-positive endocrine cell or precursor population. In certain embodiments, differentiated β cells (e.g., stem cell derived β cells) can be enriched from at least about 2- to about 20-fold as compared to a population before the chemical induction of insulin-positive endocrine cell or precursor population.

Provided herein is a method of selecting a target cell (e.g., differentiated β cells (e.g., stem cell derived β cells)) from a population of cells comprising contacting the target cell with a stimulating compound, wherein the contacting induces a selectable marker (e.g., CD49a) of the target cell to localize to a cell surface of the target cell, and selecting the target cell (e.g., differentiated β cells (e.g., stem cell derived β cells)) based on the localization of the selectable marker (e.g., CD49a) at the cell surface. In some embodiments, the selectable marker comprises CD49a. In some embodiments, the selecting the target cell is by cell sorting. In some embodiments, the selecting comprises contacting the selectable marker of the target cell with an antigen binding polypeptide when the selectable marker is localized to the surface of the target cell. In some embodiments, the antigen binding polypeptide comprises an antibody. In some embodiments, the antigen binding polypeptide binds to the CD49a. In some embodiments, the method further comprises treating the population of cells with a compound (e.g., enzyme) that removes the selectable marker from a cell surface of at least one cell of the target cell population. In some embodiments, the population of target cells is treated with the compound prior to the contacting of the target cell with the stimulating compound. In some embodiments, the compound cleaves the selectable marker from the cell surface of the at least one cell. In some embodiments, the target cell is an endocrine cell. In some embodiments, the stimulating compound comprises glucose. In some embodiments, the endocrine cell is a β cell. In some embodiments, the β cell is an SC-β cell. In some embodiments, selecting the target cell separates the target cell from the one or more cells of the population of cells.

### Pharmaceutical Compositions

In some embodiments, the present disclosure provides pharmaceutical compositions that can utilize non-native pancreatic beta cell populations and cell components and products in various methods for treatment of a disease (e.g., diabetes). Certain cases encompass pharmaceutical compositions comprising live cells (e.g., non-native pancreatic beta cells alone or admixed with other cell types). Other cases encompass pharmaceutical compositions comprising non-native pancreatic beta cell components (e.g., cell lysates, soluble cell fractions, conditioned medium, ECM, or components of any of the foregoing) or products (e.g., trophic and other biological factors produced by non-native pancreatic beta cells or through genetic modification, conditioned medium from non-native pancreatic beta cell culture). In either case, the pharmaceutical composition may further comprise other active agents, such as anti-inflammatory agents, exogenous small molecule agonists, exogenous small molecule antagonists, anti-apoptotic agents, antioxidants, and/or growth factors known to a person having skill in the art.

Pharmaceutical compositions of the present disclosure can comprise non-native pancreatic beta cell, or components or products thereof, formulated with a pharmaceutically acceptable carrier (e.g. a medium or an excipient). The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as liquids. Pharmaceutical compositions comprising living non-native pancreatic beta cells can be formulated as liquids, semisolids (e.g., gels, gel capsules, or liposomes) or solids (e.g., matrices, scaffolds and the like).

Pharmaceutical compositions may comprise auxiliary components as would be familiar to a person having skill in the art. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other anti-oxidant agents suitable for renal administration, e.g. ascorbic acid and its salts or sulfite or sodium metabisulfite may also be employed.

A buffering agent may be used to maintain the pH of formulations in the range of about 4.0 to about 8.0; so as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Pharmaceutical compositions comprising cells, cell components or cell products may be delivered to the kidney of a patient in one or more of several methods of delivery known in the art. In some cases, the compositions are delivered to the kidney (e.g., on the renal capsule and/or underneath the renal capsule). In another embodiment, the compositions may be delivered to various locations within the kidney via periodic intraperitoneal or intrarenal injection. Alternatively, the compositions may be applied in other dosage forms known to those skilled in the art, such as pre-formed or in situ-formed gels or liposomes.

Pharmaceutical compositions comprising live cells in a semi-solid or solid carrier are may be formulated for surgical implantation on or beneath the renal capsule. It should be appreciated that liquid compositions also may be administered by surgical procedures. In particular cases, semi-solid or solid pharmaceutical compositions may comprise semi-permeable gels, lattices, cellular scaffolds and the like, which may be non-biodegradable or biodegradable. For example, in certain cases, it may be desirable or appropriate to sequester the exogenous cells from their surroundings, yet enable the cells to secrete and deliver biological molecules (e.g., insulin) to surrounding cells or the blood stream. In these cases, cells may be formulated as autonomous implants comprising living non-native pancreatic beta cells or cell population comprising non-native pancreatic beta cell surrounded by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression.

In other cases, various degradable gels and networks can be used for the pharmaceutical compositions of the present disclosure. For example, degradable materials particularly suitable for sustained release formulations include biocompatible polymers, such as poly(lactic acid), poly (lactic-co-glycolic acid), methylcellulose, hyaluronic acid, collagen, and the like.

In other cases, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established.

Examples of scaffold or matrix (sometimes referred to collectively as “framework”) material that may be used in the present disclosure include nonwoven mats, porous foams, or self-assembling peptides. Nonwoven mats, for example, may be formed using fibers comprising a synthetic absorbable copolymer of glycolic and lactic acids (PGA/PLA), foams, and/or poly(epsilon-caprolactone)/poly(glycolic acid) (PCL/PGA) copolymer.

In another embodiment, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures. In many of the abovementioned cases, the framework may be molded into a useful shape. Furthermore, it will be appreciated that non-native pancreatic beta cells may be cultured on pre-formed, non-degradable surgical or implantable devices.

The matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

In some embodiments, any of the cell compositions disclosed herein (e.g., a composition comprising in vitro differentiated islet cells) further comprises a medium. In some embodiments, the medium comprises a sugar. In some embodiments, the sugar is sucrose or glucose. In some embodiments, the medium comprises the sugar at a concentration of between about 0.05% and about 1.5%. In some embodiments, the medium is a CMRL medium or wherein the medium is HypoThermosol® FRS Preservation Media.

In some embodiments, the population of cells are in a cell cluster. In some embodiments, the cell cluster is between 125-225 microns, 130-160, 170-225, 140-200, 140-170, 160-220, 170-215, or 170-200 microns in diameter.

In some embodiments, the disclosure provides for a composition comprising NKX6.1-positive, ISL1-positive cells that express lower levels of MAFA than NKX6.1-positive, ISL1-positive cells from the pancreas of a healthy control adult subject. In some embodiments, the population comprises NKX6.1-positive, ISL1-positive cells that express higher levels of MAFB than NKX6.1-positive, ISL1-positive cells from the pancreas of a healthy control adult subject. In some embodiments, the population comprises NKX6.1-positive, ISL1-positive cells that express higher levels of SIX2, HOPX, IAPP and/or UCN3 than NKX6.1-positive, ISL1-positive cells from the pancreas of a healthy control adult subject. In some embodiments, the population comprises NKX6.1-positive, ISL1-positive cells that do not express MAFA. In some embodiments, the population comprises NKX6.1-positive, ISL1-positive cells that express MAFB. In some embodiments, the population of cells is derived from stem cells in vitro. In some embodiments, the stem cells are genetically modified. In some embodiments, the stem cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the stem cells have increased expression of CD47, PDL1, HLA-G, CD46, CD55, CD59 and CTLA, relative to stem cells that are not genetically modified. In some embodiments, any of the cell markers disclosed herein (e.g., NKX6.1, PDX1, MAFA, MAFB, SIX2, HOPX, IAPP and/or UCN3) are detected by flow cytometry.

In one aspect, the present disclosure provided devices comprising a cell cluster comprising at least one pancreatic β cell. A device provided herein can be configured to produce and release insulin when implanted into a subject. A device can comprise a cell cluster comprising at least one pancreatic β cell, e.g., a non-native pancreatic β cell. A cell cluster in the device can exhibit in vitro GSIS. A device can further comprise a semipermeable membrane. The semipermeable membrane can be configured to retain the cell cluster in the device and permit passage of insulin secreted by the cell cluster. In some cases of the device, the cell cluster can be encapsulated by the semipermeable membrane. The encapsulation can be performed by any technique available to one skilled in the art. The semipermeable membrane can also be made of any suitable material as one skilled in the art would appreciate and verify. For example, the semipermeable membrane can be made of polysaccharide or polycation. In some cases, the semipermeable membrane can be made of poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, biodegradable polyurethanes, albumin, collagen, fibrin, polyamino acids, prolamines, alginate, agarose, agarose with gelatin, dextran, polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, polyethylene oxide, or any combinations thereof. In some cases, the semipermeable membrane comprises alginate. In some cases, the cell cluster is encapsulated in a microcapsule that comprises an alginate core surrounded by the semipermeable membrane. In some cases, the alginate core is modified, for example, to produce a scaffold comprising an alginate core having covalently conjugated oligopeptides with an RGD sequence (arginine, glycine, aspartic acid). In some cases, the alginate core is modified, for example, to produce a covalently reinforced microcapsule having a chemoenzymatically engineered alginate of enhanced stability. In some cases, the alginate core is modified, for example, to produce membrane-mimetic films assembled by in-situ polymerization of acrylate functionalized phospholipids, In some cases, microcapsules are composed of enzymatically modified alginates using epimerases, In some cases, microcapsules comprise covalent links between adjacent layers of the microcapsule membrane. In some embodiment, the microcapsule comprises a subsieve-size capsule comprising alginate coupled with phenol moieties. In some cases, the microcapsule comprises a scaffold comprising alginate-agarose. In some cases, the SC-β cell is modified with PEG before being encapsulated within alginate. In some cases, the isolated populations of cells, e.g., SC-β cells are encapsulated in photoreactive liposomes and alginate. It should be appreciated that the alginate employed in the microcapsules can be replaced with other suitable biomaterials, including, without limitation, polyethylene glycol (PEG), chitosan, polyester hollow fibers, collagen, hyaluronic acid, dextran with ROD, BHD and polyethylene glycol-diacrylate (PEGDA), poly(MPC-co-n-butyl methacrylate-co-4-vinylphenyl boronic acid) (PMBV) and poly(vinyl alcohol) (PVA), agarose, agarose with gelatin, and multilayer cases of these.

### Methods of Treatment

Further provided herein are methods for treating or preventing a disease in a subject. A composition comprising the cell clusters resembling endogenous pancreatic islets can be administered into a subject to restore a degree of pancreatic function in the subject. For example, the cell clusters resembling endogenous pancreatic islets can be transplanted to a subject to treat diabetes.

In some embodiments, a composition comprising cell clusters prepared according to methods disclosed herein achieve improved clinical outcomes when administered to a subject. For example, in some cases, viability of the transplanted pancreatic islets is increased compared to alternative compositions (e.g., prepared by alternate methods). In some embodiments, reduced immune infiltration is observed in response to the transplant compared to alternative compositions (e.g., prepared by alternate methods). In some embodiments, any of the cells disclosed herein are administered in a device (e.g., any of the devices disclosed herein).

The methods can comprise transplanting the cell cluster disclosed in the application to a subject, e.g., a subject in need thereof. The terms “transplanting” and “administering” can be used interchangeably and can refer to the placement of cells or cell clusters, any portion of the cells or cell clusters thereof, or any compositions comprising cells, cell clusters or any portion thereof, into a subject, by a method or route which results in at least partial localization of the introduced cells or cell clusters at a desired site. The cells or cell clusters can be implanted directly to the pancreas, or alternatively be administered by any appropriate route which results in delivery to a desired location in the subject where at least a portion of the implanted cells or cell remain viable. The period of viability of the cells or cell clusters after administration to a subject can be as short as a few hours, e.g. twenty-four hours, to a few days, to as long as several years. In some instances, the cells or cell clusters, or any portion of the cells or cell clusters thereof, can also be transadministered at a non-pancreatic location, such as in the liver or subcutaneously, for example, in a capsule (e.g., microcapsule) to maintain the implanted cells or cell clusters at the implant location and avoid migration.

A subject that can be treated by the methods herein can be a human or a non-human animal. In some cases, a subject can be a mammal. Examples of a subject include but are not limited to primates, e.g., a monkey, a chimpanzee, a bamboo, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

As used herein, the term “treating” and “treatment” can refer to administering to a subject an effective amount of a composition (e.g., cell clusters or a portion thereof) so that the subject as a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

The methods and compositions provided herein may be used to treat a subject who has, or has a risk (e.g., an increased risk) of developing a disease. In some cases, the disease is diabetes, including, but not limited to, type I diabetes, type II diabetes, type 1.5 diabetes, prediabetes, cystic fibrosis-related diabetes, surgical diabetes, gestational diabetes, and mitochondrial diabetes The disease may also be a diabetes complication, including heart and blood vessel diseases, diabetic nephropathy, diabetic neuropathy, diabetic retinopathy, foot damages, and hearing damages.

The methods can comprise transplanting the cell cluster to a subject using any means in the art. For example the methods can comprise transplanting the cell cluster via the intraperitoneal space, renal subcapsule, renal capsule, momentum, subcutaneous space, or via pancreatic bed infusion. For example, transplanting can be subcapsular transplanting, intramuscular transplanting, or intraportal transplanting, e.g., intraportal infusion. Immunoprotective encapsulation can be implemented to provide immunoprotection to the cell clusters.

In some embodiments, any of the cells, clusters, or compositions disclosed herein may be utilized in methods other than methods of treatment. In some embodiments, the cells, clusters or compositions have utility for assessing the development of stem, pluripotent, multipotent, or unipotent cells in vitro or in vivo. For example, in some embodiments, the disclosure provides for compositions comprising cells (e.g., PDX.1⁺; NKX6.1⁺ pancreatic progenitor cells or insulin⁺ pancreatic endocrine progenitor cells) and any one or more of the reagents disclosed herein (e.g., any one or more of the metabolites disclosed herein) to study the effect (e.g., the expression of specific genes) in the cells in response to the reagents.

### Devices

In some embodiments, the disclosure provides for a device comprising any of the cells, clusters or compositions disclosed herein. Exemplary devices are described in WO2019068059A1, WO2020206150, WO 2020/206150, and WO 2020/206157, each of which is incorporated by reference herein it its entirety.

In some embodiments, the device is configured to produce and release insulin when implanted into a subject. In some embodiments, the device is a semipermeable membrane, wherein the semipermeable membrane is configured to retain any of the cells disclosed herein in the device and permit passage of insulin produced by the cells out of the device.

In a certain aspect, described herein, is a cell housing device, comprising: a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment having a surface area to the volume ratio of at least about 40 cm⁻¹, and wherein the enclosed compartment provides a volume for housing a cell within the device.

In some embodiments, the compartment comprises a single continuous open space. In some embodiments, the volume is about 8 μL to about 1,000 μL. In some embodiments, the device has at least one of a length and a width of about 0.25 cm to about 3 cm. In some embodiments, the device has a thickness of at least about 300 m. In some embodiments, the plurality of channels are generally perpendicular with respect to the first membrane. In some embodiments, the plurality of channels are arranged in a rectilinear array. In some embodiments, the plurality of channels are arranged in a polar array. In some embodiments, the channel has an average diameter of about 400 m to about 3,000 m. In some embodiments, the diameter is measured at a narrowest point in the channel. In some embodiments, a center of each channel is separated from the center of another channel by a distance of about 75 m to about 500 m. In some embodiments, the channel has a height to diameter ratio of at least about 0.2. In some embodiments, the device has a number of channels per area along a transverse plane is greater than about 50/cm². In some embodiments, at least one of the first membrane and the second membrane comprise a plurality of nodes interconnected by a plurality of fibrils. In some embodiments, at least one of the first membrane and the second membrane comprise PVDF, PTFE, ePTFE, PCL, PE/PES, PP, PS, PMMA, PLGA, PLLA, or any combination thereof. In some embodiments, the device further comprises an opening through the first membrane and the second membrane within the channel. In some embodiments, the opening has a concentricity with respect to the channel of at most 25% the diameter of the channel. In some embodiments, the device further comprises a frame configured to receive the device. In some embodiments, the frame is configured to receive a plurality of cell housing devices. In some embodiments, the frame comprises a flexing mechanism configured to prevent buckling of the cell housing device.

In some embodiments, the device further comprises a cell population. In some embodiments, the cell population is an insulin secreting population. In some embodiments, the cell population is a stem cell derived cell that are capable of glucose-stimulated insulin secretion (GSIS). In some embodiments, the device further comprises a coating comprising a hydrophilic polymer. In some embodiments, the device has an insulin diffusion coefficient of about 2×10⁻⁶ cm²/s to about 1×10⁻⁵ cm 2/s. In some embodiments, the device has a maximum insulin diffusion distance of less than about 150 m. In some embodiments, the first membrane and the second membrane are fused with a fusion peel force of at least about 0.4 N. In some embodiments, at least one of the first membrane and the second membrane are semi-permeable. In some embodiments, the semi-permeability of the first membrane, the second membrane, or both is configured to protect the cell from an immune attack. In some embodiments, the semi-permeability of the first membrane, the second membrane, or both is configured to protect the cell from an immune attack in the absence of an immune suppression therapy. In some embodiments, at least one of the first membrane and the second membrane are configured to enable vascularization of the cell within the device. In some embodiments, at least one of the first membrane and the second membrane are configured to enable vascularization of the cell within the device in absence of an immune suppression therapy.

Another aspect provided herein is a cell housing device, comprising: a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment wherein the enclosed compartment provides a volume for housing 1 million to 1 billion insulin producing cells within the device and wherein said membrane allows for diffusion of insulin from the device while retaining the insulin producing cells within the device.

In some embodiments, the device is an implantable microencapsulation device. In some embodiments, the implantable macroencapsulation device comprises: a first outer membrane; a second outer membrane; and a first semipermeable membrane attached between the first outer membrane and the second outer membrane; wherein the first semipermeable membrane and the first outer membrane are connected to form a primary compartment configured to provide a primary compartment for housing a population of cells; wherein the first semipermeable membrane and the second outer membrane are connected to form a secondary compartment; wherein the population of cells comprises pancreatic progenitor cells, endocrine cells, or beta cells, or any combination thereof; and wherein the device comprises a plurality of through holes through the first outer membrane, the second outer membrane, and the first semipermeable membrane. In some embodiments, the first outer membrane, the second outer membrane, and the first semipermeable membrane are configured to block passage of said population of cells out of the device.

In some embodiments, the device further comprises a second semipermeable membrane attached between the first semipermeable membrane and the second outer membrane to form a tertiary compartment between the primary compartment and the secondary compartment. In some embodiments, a hydraulic permeability of the first semipermeable membrane is greater than a hydraulic permeability of the first outer membrane, a hydraulic permeability of the second outer membrane, or both. In some embodiments, a hydraulic permeability of the first semipermeable membrane is greater than the hydraulic permeability of the first outer membrane, the hydraulic permeability of the second outer membrane, or both by at least about 25%. In some embodiments, a hydraulic permeability of the first semipermeable membrane is greater than a hydraulic permeability of the second semipermeable membrane. In some embodiments, a hydraulic permeability of the first semipermeable membrane is less than a hydraulic permeability of the second semipermeable membrane. In some embodiments, a porosity of the first semipermeable membrane is greater than a porosity of the first outer membrane, a porosity of the second outer membrane, or both. In some embodiments, a porosity of the first semipermeable membrane is greater than the porosity of the first outer membrane, the porosity of the second outer membrane, or both by at least about 25%. In some embodiments, a porosity of the first semipermeable membrane is greater than a porosity of the second semipermeable membrane. In some embodiments, a porosity of the first semipermeable membrane is less than a porosity of the second semipermeable membrane. In some embodiments, a flux of the first semipermeable membrane for a given material and bias (e.g. concentration gradient and/or pressure differential) is greater than a flux of the first outer membrane, a flux of the second outer membrane, or both for the same material and bias. In some embodiments, the flux of the first semipermeable membrane for a given material and bias (e.g. concentration gradient and/or pressure differential) is greater than the flux of the first outer membrane, the flux of the second outer membrane, or both by at least about 25% for the same material and bias. In some embodiments, a flux of the first semipermeable membrane for a given material and bias (e.g. concentration gradient and/or pressure differential) is greater than a flux of the second semipermeable membrane for the same material and bias. In some embodiments, a flux of the first semipermeable membrane for a given material and bias (e.g. concentration gradient and/or pressure differential) is less than a flux of the second semipermeable membrane for the same material and bias. In some embodiments, the device further comprises a primary port in fluid communication with the primary compartment, a secondary port in fluid communication with the secondary compartment, or any combination thereof. In some embodiments, the device further comprises a primary port in fluid communication with the primary compartment, a secondary port in fluid communication with the secondary compartment, a tertiary port in fluid communication with the tertiary compartment, or any combination thereof. In some embodiments at least one of the primary port the secondary port, or the tertiary port are sealable or re-sealable.

Another aspect provided herein is an implantable macroencapsulation device comprising a primary compartment configured to house one or more cells, and a secondary compartment, wherein the primary compartment and the secondary compartment are separated by a first semipermeable membrane, wherein the secondary compartment and the first semipermeable membrane are configured to i) filter a filtrate from the primary compartment, or ii) provide an ancillary agent to the one or more cells within the primary compartment, or both i) and ii); and wherein said one or more cells are encapsulated within said device from a range of about 10³ to about 10⁶ cells per μL of volume.

In some embodiments, the device further comprises a tertiary compartment, wherein the tertiary compartment and the secondary compartment are separated by a second semipermeable membrane, wherein the second semipermeable membrane is configured to i) filter a filtrate from the tertiary compartment, or ii) provide an ancillary agent to the one or more cells within the tertiary compartment, or both i) and ii). In some embodiments, the device further comprises at least one of a primary port in fluid communication with the primary compartment, or a secondary port in fluid communication with the secondary compartment. In some embodiments, the device further comprises at least one of a primary port in fluid communication with the primary compartment, a secondary port in fluid communication with the secondary compartment, or a tertiary port in fluid communication with the tertiary compartment. In some embodiments at least one of the primary port, the secondary port, or the tertiary port are sealable or re-sealable. In some embodiments, wherein the device comprises a plurality of through holes extending from one side of the device to an opposing side of the device through the layered membranes.

In some embodiments, one or more of the through holes are surrounded by a bonded portion of the membranes to form a seal. In some embodiments, the device comprises three or more seals. In some embodiments, the device comprises two or more self-intersecting seals. In some embodiments, the device comprises two or more elliptical seals. In some embodiments the seal is formed by an adhesive, an epoxy, a weld, any combination thereof, and/or any other appropriate bonding methods. In some embodiments, the first semipermeable membrane is configured to block passage of said one or more cells. In some embodiments, the primary compartment and the secondary compartment are configured to block passage of said one or more cells. In some embodiments the primary compartment, the secondary compartment, and the tertiary compartment are configured to block passage of said one or more cells.

Another aspect provided herein is a method, comprising: providing a macroencapsulation device comprising a primary compartment configured to house one or more cells, and a secondary compartment, wherein the primary compartment and the secondary compartment are separated by a first semipermeable membrane, wherein the secondary compartment and the semipermeable membrane are configured to i) filter a filtrate from the primary compartment, or ii) provide an ancillary agent to the one or more cells within the primary compartment, or both i) and ii); pre-vascularizing the macroencapsulation device; loading one or more cells into the primary compartment; and applying a pressure to the secondary compartment to remove a filtrate from the primary compartment.

In some embodiments the filtrate is removed from the primary compartment. In some embodiments, the method further comprises administering an ancillary agent into the primary compartment, the secondary compartment, or both. In some embodiments the ancillary agent comprises a drug, an oxygen generating substance, an anti-coagulant, a nutrient, or any combination thereof. In some embodiments administering the ancillary agent is performed after applying a negative pressure to the secondary compartment, though any method of providing a desired pressure differential of the secondary compartment relative to another compartment of the macroencapsulation device may also be used. In some embodiments, the method further comprises, inflating the primary compartment, the secondary compartment, or both. In some embodiments inflating the primary compartment, the secondary compartment, or both is performed before the prevascularizing of the macroencapsulation device. In some embodiments, the method further comprises, sealing the primary compartment, the secondary compartment, or both. In some embodiments, the method further comprises resealing the primary port, the secondary port, or both. In some embodiments, the housing further comprises a tertiary compartment separated from the secondary compartment by a second semipermeable membrane, and wherein the method further comprises loading one or more cells into the tertiary compartment. In some embodiments, the method further comprises administering an ancillary agent into the primary compartment, the secondary compartment, or the tertiary compartment, or any combination thereof. In some embodiments, the method further comprises inflating the primary compartment, the secondary compartment, or the tertiary compartment, or any combination thereof. In some embodiments, the method further comprises sealing the primary compartment, the secondary compartment, or the tertiary compartment, or any combination thereof. In some embodiments, the method further comprises resealing the primary port, the secondary port, the tertiary compartment, or any combination thereof.

Another aspect provided herein is a method, comprising: providing a macroencapsulation device comprising: a first outer membrane; a second outer membrane; and a first semipermeable membrane attached between the first outer membrane and the second outer membrane; wherein the first semipermeable membrane and the first outer membrane are connected to form a primary compartment configured for housing a population of cells; and wherein the first semipermeable membrane and the second outer membrane are connected to form a secondary compartment; pre-vascularizing the macroencapsulation device; loading one or more cell into the primary compartment; and applying a pressure differential to the secondary compartment to remove a filtrate from the primary compartment.

In some embodiments the filtrate is removed from the primary compartment. In some embodiments, the method further comprises administering an ancillary agent into the primary compartment, or the secondary compartment, or both. In some embodiments the ancillary agent comprises a drug, an oxygen generating substance, an anti-coagulant, a nutrient, or any combination thereof. In some embodiments administering the ancillary agent is performed after the applying a negative pressure or other pressure differential to the secondary compartment. In some embodiments, the method further comprises inflating the primary compartment, or the secondary compartment, or both. In some embodiments inflating the primary compartment, or the secondary compartment, or both is performed before prevascularizing of the macroencapsulation device. In some embodiments, the method further comprises sealing the primary compartment, or the secondary compartment, or both. In some embodiments, the method further comprises resealing the primary port, the secondary port, or both. In some embodiments, the macroencapsulation device further comprises a tertiary compartment separated from the secondary compartment by a second semipermeable membrane, and wherein the method further comprises loading one or more cell into the tertiary compartment. In some embodiments, the method further comprises administering an ancillary agent into the primary compartment, the secondary compartment, or the tertiary compartment, or any combination thereof. In some embodiments the filtrate is removed from the primary compartment, the tertiary compartment, or both. In some embodiments, the method further comprises inflating the primary compartment, the secondary compartment, the tertiary compartment, or any combination thereof. In some embodiments, the method further comprises sealing the primary compartment, the secondary compartment, or the tertiary compartment, or any combination thereof. In some embodiments, the method further comprises resealing the primary port, the secondary port, the tertiary compartment, or any combination thereof.

### EXAMPLES

The examples below further illustrate the described embodiments without limiting the scope of this disclosure.

### Example 1. Modified Stage 5 Culture Conditions

Improved methods of generating SC-β cells could result in more effective therapeutic products (e.g., SC-β cells with improved functionality), improved methods of manufacturing SC-islets for human therapeutic use (e.g., higher cell yields), or a combination thereof. Studies were conducted to identify modifications to stage 5 culture conditions that have beneficial effects on the resulting SC-β cells.

Stage 5 cells may be obtained in accordance with the Version A protocol described in Example 1 of U.S. patent application Ser. No. 17/390,839, which is incorporated by reference herein in its entirety. A set of media additives was devised for addition during stage 5 (PDX.1⁺; NKX6.1⁺ pancreatic progenitors) of the differentiation protocol disclosed herein. The set of additives was designed to improve the fitness and metabolic flexibility of the differentiating cells and the resulting SC-β cells by targeting specific aspects of cellular metabolism. The media additives included acetate, p hydroxybutyrate, taurine, formate, biotin, and glutamine, and can be referred to collectively as “MQ” herein. The glutamine utilized was in a non-dipeptide form to increase bioavailability, e.g., to avoid the need for processing by cell peptidases. MQ media in this example comprises 1 mM acetate, 200 nM R hydroxybutyrate, 90 μM taurine, 50 μM formate, 800 nM biotin, and 4 mM L-glutamine, all added to a stage 5 medium of the disclosure containing Sant1, retinoic acid and betacellulin for the first two days and XXI, Alk5i, GC-1, LDN-193189, thiazovivin, staurosporine, and DZNEP for seven days. After addition of MQ, media was filtered and stored at 4° C., with a maximum storage duration of 2-4 days prior to use.

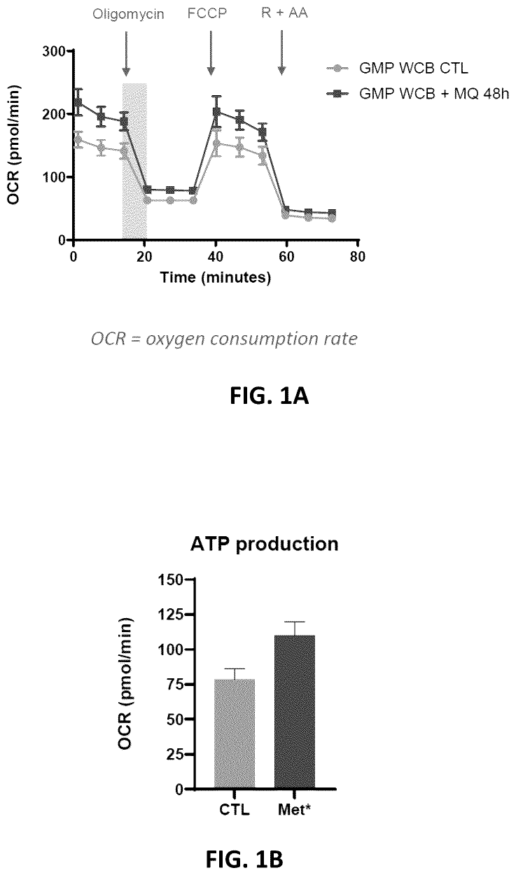

In an initial study, the effect of MQ on human embryonic stem cell (hESC) metabolism was evaluated in a Seahorse assay (Agilent). hESCs were cultured for 48 hours with or without MQ. hESCs cultured in the presence of MQ exhibited an increased basal metabolic rage (FIG. 1A, e.g., see left side of graph), and increased ATP-linked oxygen consumption (FIG. 1B), indicating the MQ can alter intracellular metabolism. Additionally, cultures incubated with MQ exhibited increased cell density, suggesting enhanced growth (FIG. 1C). When glutamine and acetate were removed from the MQ cocktail, cell density was reduced compared to the complete set of additives (FIG. 1C), suggesting that glutamine and acetate contribute to the observed effects of MQ on the cells.

Next, the ability of MQ to enhance SC-β cell differentiation and functionality was evaluated. Stage 4 pancreatic progenitor 2 cells were differentiated from stem cells as described herein. During stage 5, the pancreatic progenitor 2 cells are further differentiated, inter alia, into pancreatic endocrine β cells (“SC-β cells”).

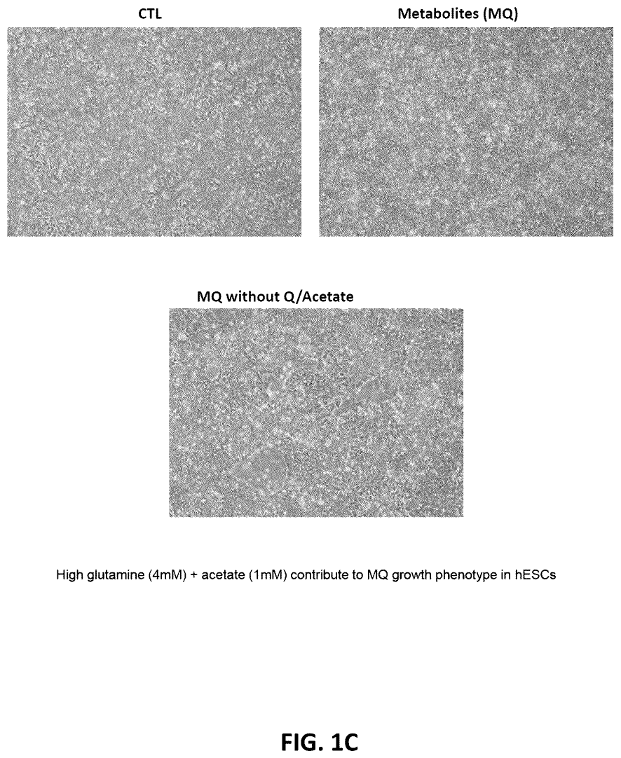

MQ was added to media throughout stage 5, and the impact on SC-β cell differentiation was evaluated. FIG. 2A provides illustrative scatterplots from samples evaluated at the conclusion of stage 5, for cultures incubated with MQ (+Metabolites) or in the absence of MQ (control). SC-β cells are shown in the upper right quadrant (Nkx6.1+/Isl1+). As shown in the figure, the addition of MQ to media throughout stage 5 boosted the percent of SC-β cells substantially, from 25.9% in the control culture to 38.1% in the culture with MQ. FIG. 2B quantifies the percentage of Nkx6.1+/Is11+SC-β cells obtained at the end of stage 5 from repeats conducted in the control condition (without MQ), showing that the SC-3% achieved in the absence of MQ is reproducibly lower than that observed with MQ in FIG. 2A. These data suggest that the addition of MQ during stage 5 can enhance SC-β cell differentiation.

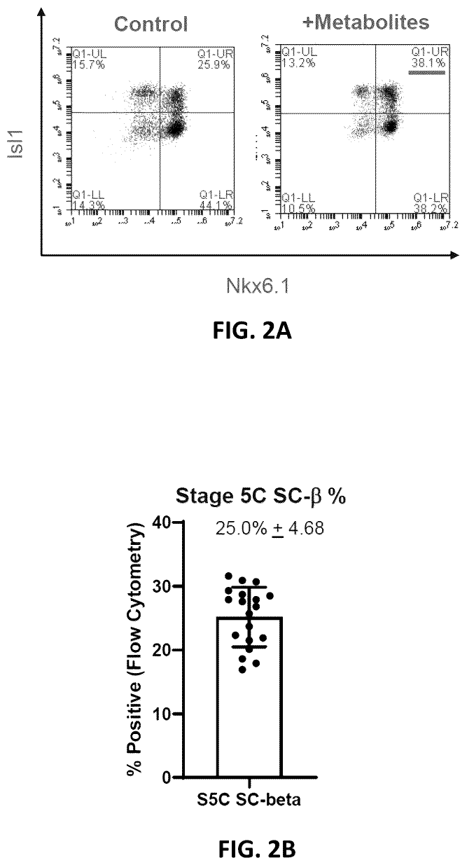

FIG. 3 provides illustrative flow cytometry results from a separate experiment with MQ added (or absent) throughout stage 5. In this case, the percent of Nkx6.1+/Is11+SC-β cells was increased from 29.8% in the control condition to 41.6% in the presence of MQ. Additionally, the percentage of Nkx6.1-/Is11-(double negative) cells was decreased from 26.4% in the control to 18.4% in the presence of MQ, consistent with more cells undergoing differentiation. In this assay, a third condition was evaluated, with the MQ additives present in the media except for acetate and glutamine (-acetate, -Q). 34.3% of cells in that condition were Nkx6.1+/Is11+, suggesting that acetate and glutamine contribute to the increase in SC-β cell differentiation observed with MQ.

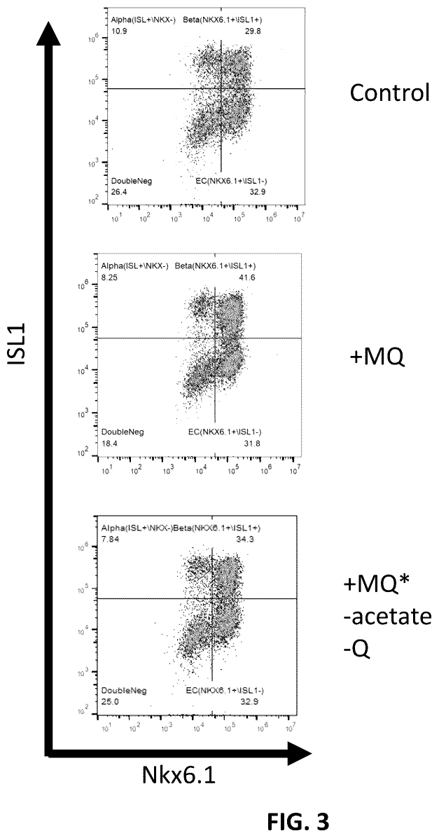

FIG. 4 provides summary data from two experiments comparing the percent of SC-β cells present at the end of stage 5 following incubation in the presence or absence of MQ throughout stage 5.

Additional studies focused on whether the addition of MQ during stage 5 affects cell characteristics downstream in stage 6. Cells generated in the presence or absence of MQ as above were dissociated into individual cells, cryopreserved, and thawed. The thawed individual cells were then differentiated into β cell clusters through a stage 6 differentiation. Stage 6 media and conditions were the same, so observed differences should be attributable to the presence of MQ in stage 5.

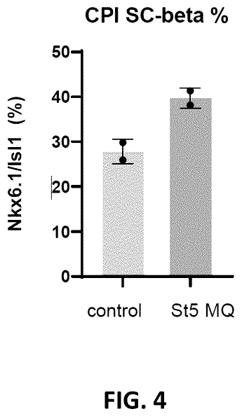

When the percentage of Nkx6.1+/Isl1+SC-β cells was evaluated on day 4 of stage 6, a higher proportion of SC-β cells was observed for the cultures that were treated with MQ in stage 5. FIG. 5 provides illustrative flow cytometry data from stage 6 day 4, showing that 47.3% of cells were Nkx6.1+/Isl1+ for the culture where MQ was present in stage 5 (+Metabolites), compared to 37% for the control culture.

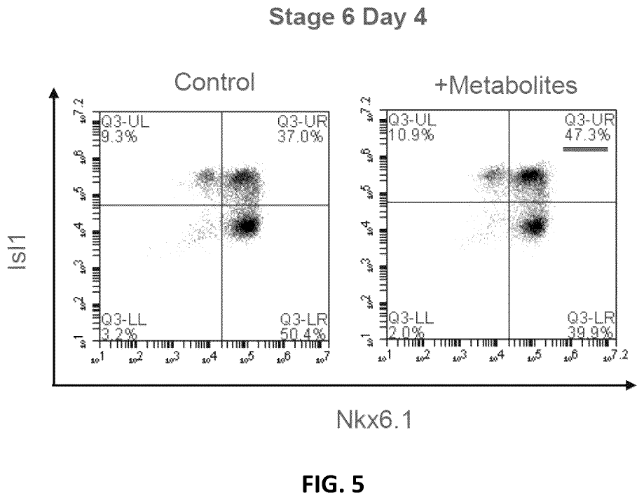

The yield of total cells and of SC-β cells was also higher for cultures in which MQ was present in stage 5. FIG. 6A demonstrates the percent recovery of all cells on days 4 and 10 of stage 6, relative to the number of viable cells seeded at the beginning of stage 6. Two counts were performed each day (day 4 and day 10) and were averaged for each condition. The dots represent the averages. FIG. 6B quantifies the number of SC-β cells on day 4 of stage 6, calculated from the total cell yield times the percent of cells that were SC-β cells.

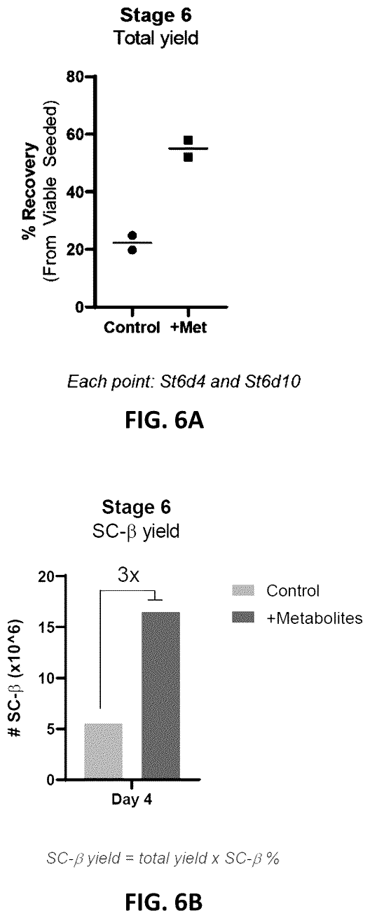

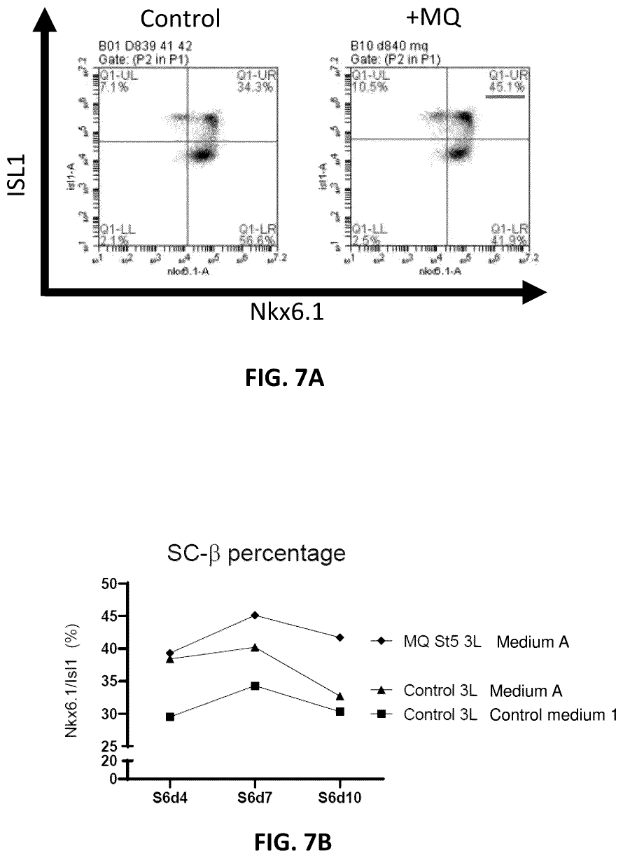

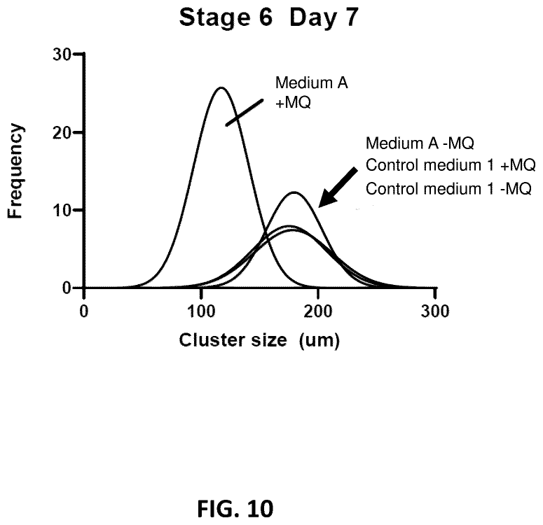

A similar study was conducted in which the effects of MQ addition on cells during stage 6 was further characterized. In this study, after thawing the cells were cultured in either Control medium 1 or medium A during stage 6 (e.g., control medium 1 or medium A as characterized in FIG. 11). Once again, a higher percentage of SC-β cells was observed for cells that were cultured with MQ during stage 5. FIG. 7A shows illustrative flow plots of cells analyzed on day 7 of stage 6, showing that 45.1% of cells were Nkx6.1+/Isl1+ for the culture where MQ was present in stage 5, compared to 34.3% for cells not incubated with MQ. FIG. 7B shows the percentage of Nkx6.1+/Isl1+ cells on days 4, 7, and 10 of stage 6, indicating that the high percent of SC-β cells observed with stage 5 MQ treatment is maintained over time (MQ St5). FIG. 8 illustrates the percent recovery (yield) of all cells on days 4, 7, and 10 of stage 6, relative to the number of viable cells seeded at the beginning of stage 6. As in the earlier study, higher yield was observed for cells that were incubated with MQ in stage 5 (MQ St5).

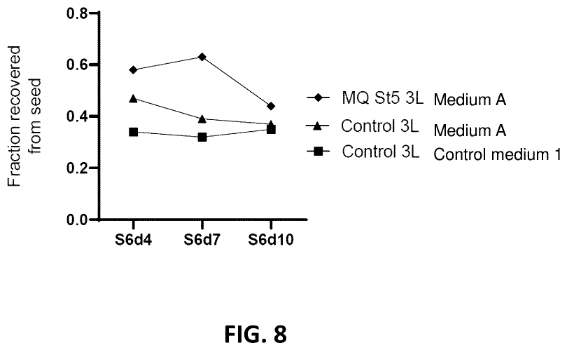

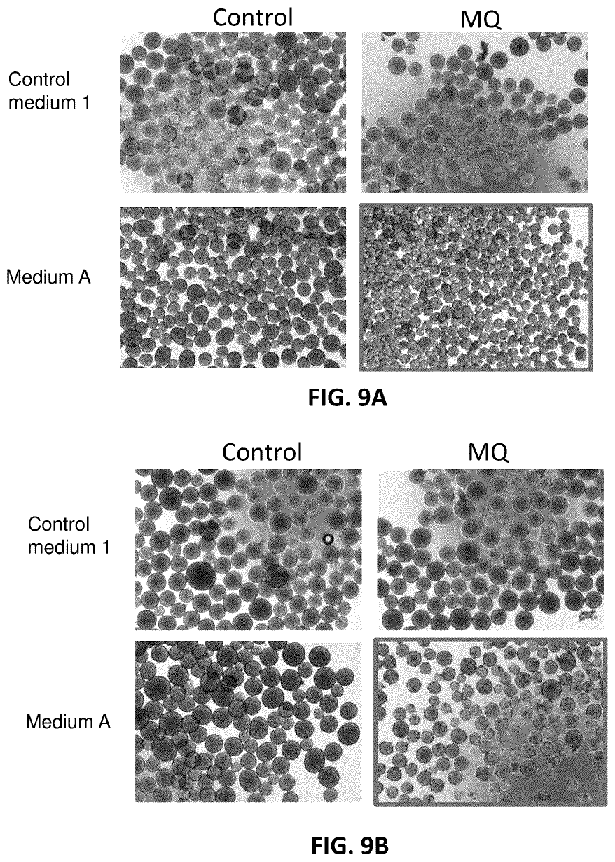

Cells that had been incubated with MQ in stage 5, then thawed into medium A in stage 6, formed smaller clusters when observed on days 4 (FIG. 9A) and 7 (FIG. 9B) of stage 6. FIG. 10 quantifies cluster size on day 7 of stage 6, showing cluster size is smaller for the cells incubated with MQ in stage 5, then thawed into medium A in stage 6. Smaller cluster diameter can be a desirable characteristic for SC-β cells clusters of the disclosure, for example, to allow effective diffusion of molecules (e.g., nutrition and gas) from surrounding environment into the core of the cell cluster.

These data show that the presence of MQ during stage 5 of a differentiation protocol of the disclosure can result in multiple beneficial effects during stage 5 and stage 6 of methods of the disclosure, for example, a higher proportion of SC-β cells, higher yields of total cells and SC-β cells in stage 6, maintenance of SC-β cell and total cell yields over time, and smaller cluster diameters.

### Example 2. Stage 6 Culture Conditions with Addition of Metabolites

Insulin-positive endocrine cell clusters were differentiated from stem cells. The insulin-positive endocrine cell clusters were dissociated into individual cells, cryopreserved, and thawed. The thawed individual cells were then differentiated into β cell clusters through a stage 6 differentiation, with a comparison of different media during stage 6.

A modified stage 6 medium comprising DMEM/F12 base media, 10 μM zinc, metabolites, S5d6 factors (for days 1-4 of stage 6 only) (Alk5i (10 μM), GC-1 (1 μM), LDN-193189 (100 nM), thiazovinin (2.5 μM), SSP (3 nM), DZNEP (100 nM)), and HSA (0.05% for days 1-4, and 1% for days 5-11) (“medium A”) was made (FIG. 11). A select number of metabolites were included in medium A, including: glutamate, acetate, R hydroxybutyrate, L-carnitine, taurine, formate, and biotin. A control medium A lacking the metabolites (lacking glutamate, acetate, R hydroxybutyrate, L-carnitine, taurine, formate, and biotin) was also formulated.

Stage 6 incubations were conducted using the medium A with the metabolites, the medium A lacking the metabolites, control medium 1, or control medium 2, as shown in FIG. 11.

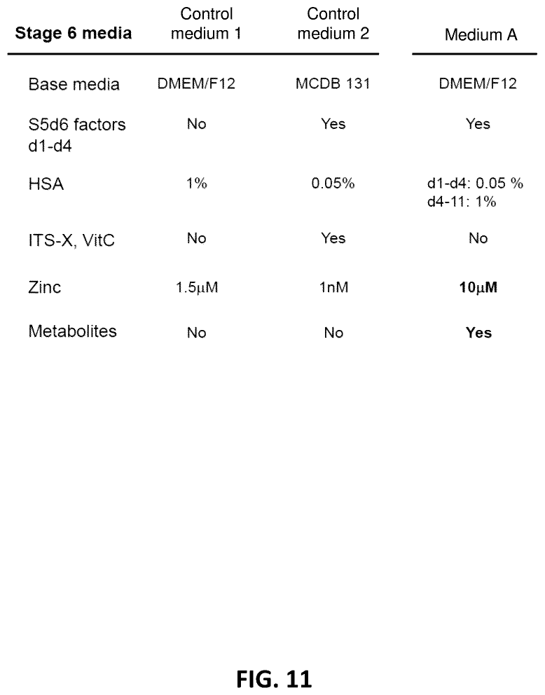

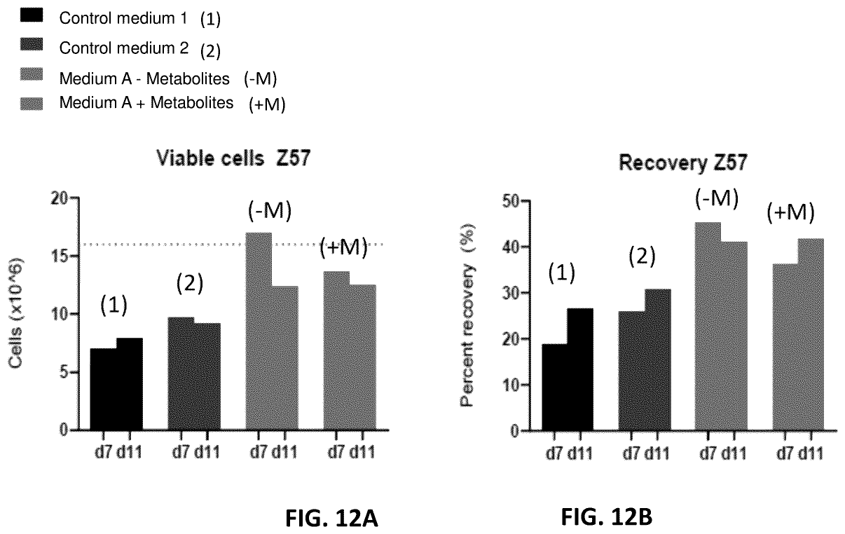

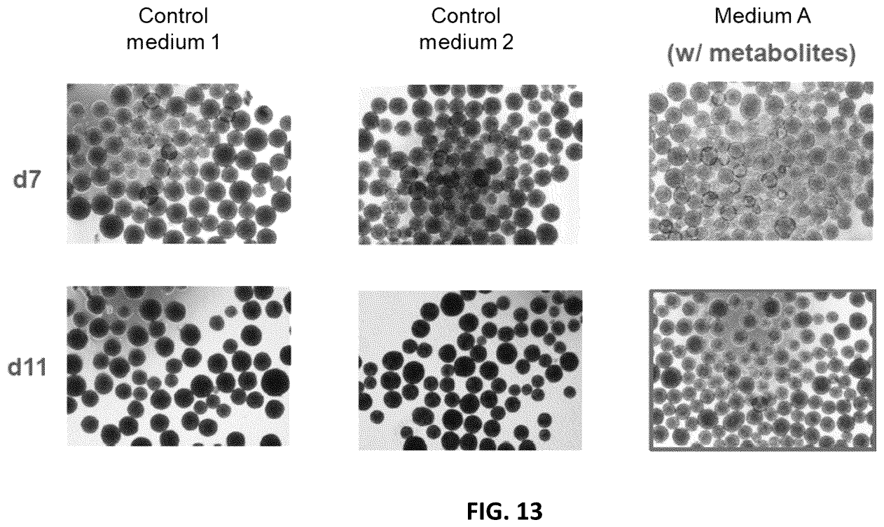

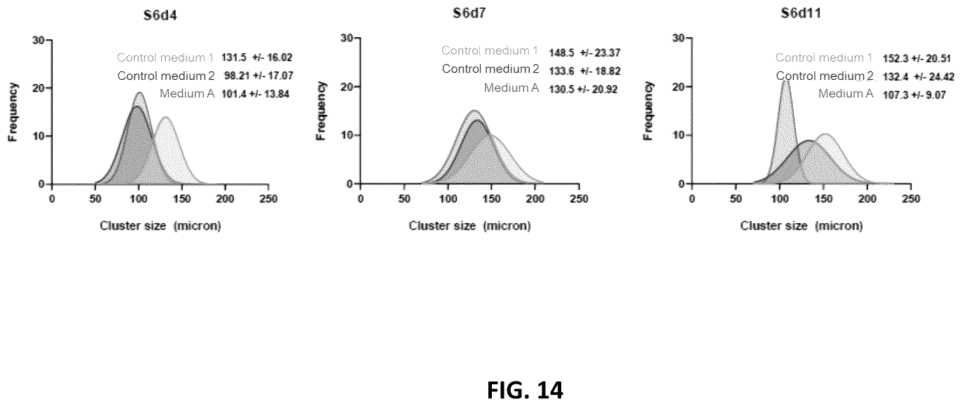

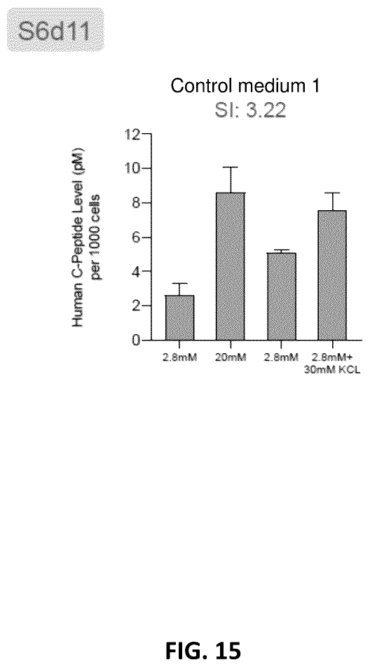

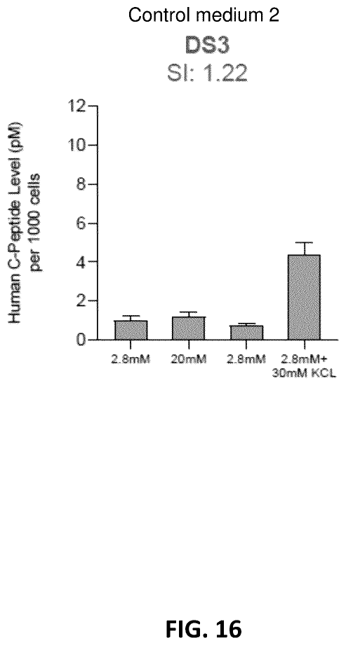

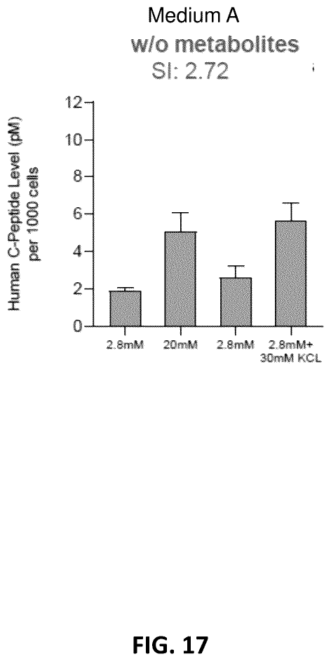

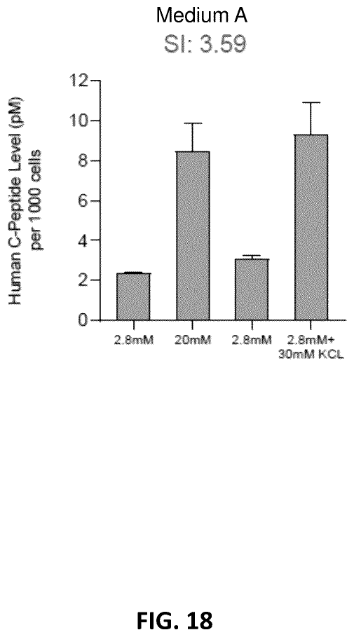

As shown in FIG. 12B, medium A improves stage 6 cells recovery (40% recovery at S6d11). Furthermore, medium A cell clusters are smaller (110 microns by S6d11) and exhibit more homogeneity through stage 6 as compared to control medium 1 or control medium 2 (FIG. 13, FIG. 14). SC-islets differentiated in control medium 2 do not exhibit GSIS as is observed in SC-islets differentiated in control medium 1 (FIG. 15, FIG. 16); while SC-islets cultured in medium A without metabolites exhibit improved GSIS at S6d11 (FIG. 17, FIG. 15). However, SC-islets cultured in medium A with metabolites exhibit GSIS similar to SC-islets cultured in control medium 1 (FIG. 18, FIG. 15). The results indicate that the use of medium A in stage 6 results in smaller and more homogenous SC-islets, higher cell recovery, higher cell yield through S6d11, and a better GSIS profile throughout stage 6 (FIG. 19).

### Example 3. Modified Stage 6 Culture Conditions with Metabolites Added for Days 1-4

A variation of the protocol of Example 2 was tested in which metabolites were only added to the cells for days 1-4 of stage 6. This regimen can be referred to as “regimen 1.” The formulations of media used in regimen 1 during stage 6 are shown in Table 1. Media was changed on day 4, day 7, and day 10 of stage 6. The metabolites and small molecules were included in media or days 1-4, but were not included in media following the media change on day 4 onward.

TABLE 1

| | | Stage 6 days 1-4 | | Stage 6 days 4-11 |
|---|---|---|---|---|
| Basal media | | DMEM/F12 | | DMEM/F12 |
| | | w/glutamine | | w/glutamine |
| Media | HSA | 0.05% | | 1% |
| supplements | ZnSO4 | 10 | μM | 10 μM |
| Small | Alk5i | 10 | μM | — |
| molecules | GC-1 | 1 | μM | — |
| | LDN-193189 | 100 | nM | — |
| | thiazovivin | 2.5 | μM | — |
| | SSP | 3 | nM | — |
| | DZNEP | 100 | nM | — |
| Metabolites | L-glutamate | 0.5 | mM | — |
| | L-carnitine | 40 | μM | — |
| | Taurine | 90 | μM | — |
| | Acetate | 160 | nM | — |
| | B-hydroxybutyrate | 200 | nM | — |
| | Biotin | 800 | nM | — |
| | Formate | 50 | μM | — |

Cells generated by regimen 1 were compared to cells generated using regimen 2, shown in Table 2. Regimen 2 lacks the metabolites that were added to the media in regimen 1 for days 1-4.

TABLE 2

| | | Stage 6 days 1-4 | | Stage 6 days 4-11 |
|---|---|---|---|---|
| Basal media | | MCDB 131 | | MCDB 131 |
| Media | HSA | 0.05% | | 0.05% |
| supplements | Vitamin C | 0.25 | mM | 0.25 mM |
| Small molecules | Alk5i | 10 | μM | — |
| | GC-1 | 1 | μM | — |
| | LDN-193189 | 100 | nM | — |
| | thiazovinin | 2.5 | μM | — |
| | SSP | 3 | nM | — |
| | DZNEP | 100 | nM | — |

SC-islets generated using either regimen 1 or regimen 2 were harvested and formulated into membrane-bound encapsulation devices similar to the cell housing devices described in WO2019068059A1 and WO2020206150, which are incorporated herein in their entirety. The devices were implanted into the RNU rat (a model of foreign body response), and diabetic mice, to evaluate stability and efficacy of the SC-islets in vivo.

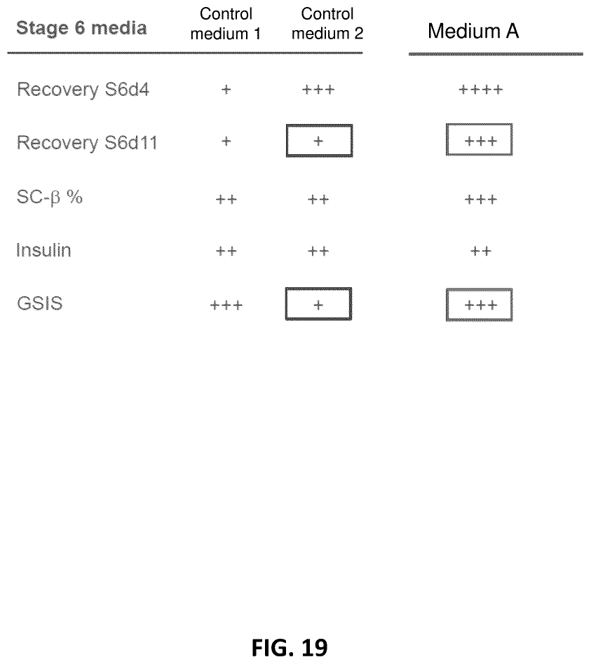

A nude rat model was used to evaluate viability of the SC-islet cells after transplantation, and certain immune reactions to the device and implanted cells. Cells generated using regimen 1 in stage 6 exhibited higher viability compared to cells generated using regimen 2, when evaluated 6 weeks post-implant (FIG. 20A). Similar viability results were observed for regimen 2 when evaluated 13 weeks post-implant (FIG. 20B). These results suggest that regimen 1 can result in improved cell survival upon in vivo administration of SC-islets to a recipient subject, which may enhance therapeutic efficacy.

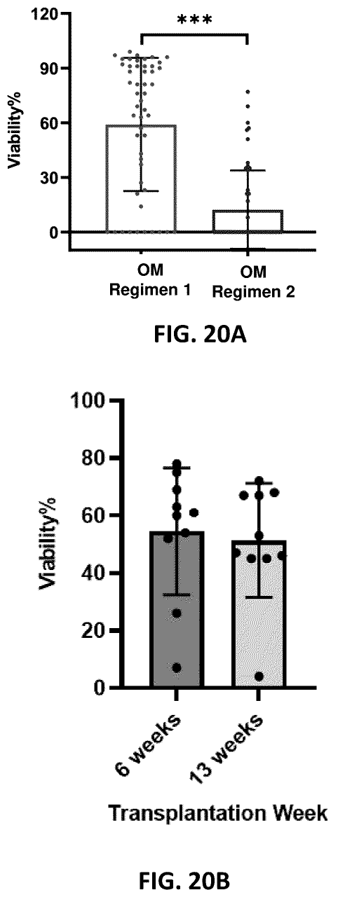

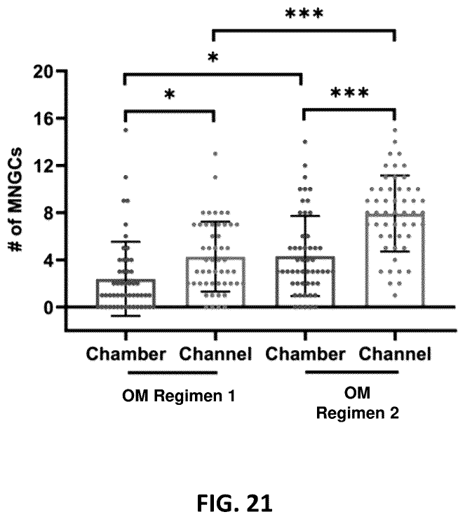

Additionally, reduced immune infiltrate and fewer multi nuclear giant cells (MNGs) were observed at 6 weeks post-implant for cells generated using regimen 1 (FIG. 21 and FIG. 22). The chambers represent the immediate area around the encapsulated cells, while the channels are the transvascularizing elements that support angiogenesis. Without wishing to be bound by theory, these results may be indicative of reduced immunogenicity of SC-islets generated by methods that include regimen 1 at stage 6, or elements present in media utilized in regimen 1.

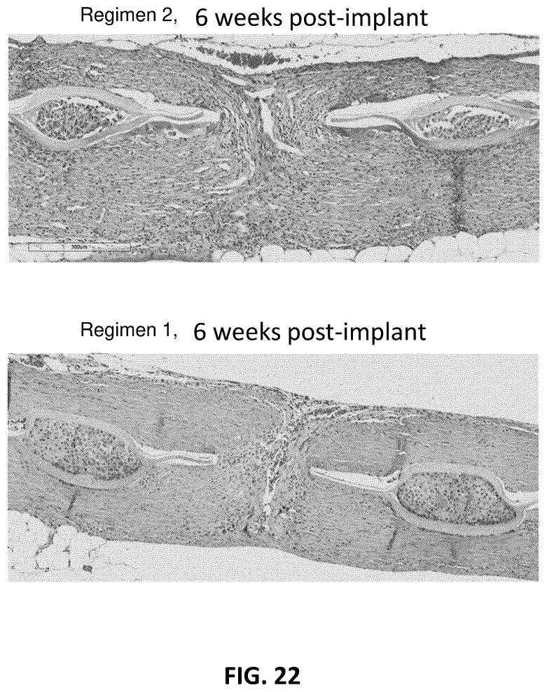

To study efficacy, a diabetic NOD Scid Gamma (NSG) mouse model was used. Animals received one (1×) or two (2×) devices loaded with SC-islets generated using the regimen 1 or regimen 2 during stage 6 of differentiation. Each device was loaded with 4.75×10⁶ cells, resulting in doses of 4.75×10⁶ or 9.5×10⁶ cells for animals implanted with one or two devices, respectively. FIG. 23 illustrates the non-fasted blood glucose levels of groups of mice over time. Animals that received two devices (2×) maintained euglycemia by ˜8 weeks post-implant. Animals that received one device (1×) exhibit reduced blood glucose levels by ˜3 weeks post-implant, but did not maintain blood glucose levels below 180 mg/dL in this experiment, however lower blood glucose levels were observed at late timepoints for animals that received one device loaded with SC-islets generated using the regimen 2. These results show that SC-islets generated my methods of the disclosure can exhibit efficacy in reducing blood glucose levels in diabetic subjects in vivo.

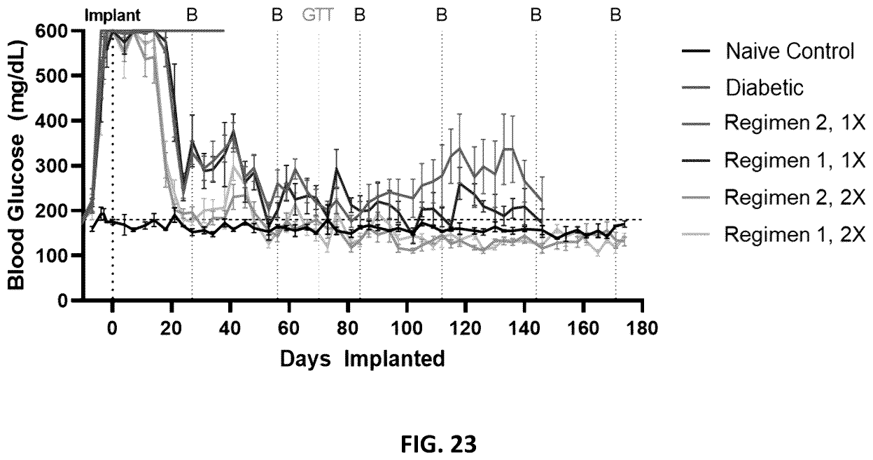

Human C-peptide levels were also monitored over time as an indicator of insulin production. FIG. 24A illustrates non-fasted human C-peptide levels in animals over time. In contrast to naive control and diabetic control mice, in which C-peptide levels were undetectable, increases in C-peptide were observed in all experimental groups following implant, with sustained levels observed for animals that received one or two devices (CAD refers to a cell-housing device). FIG. 24B compares fasted levels of human C-peptide measured on day 153 and non-fasted levels of C-peptide measured on day 112 post-transplant, and includes data for animals that received one or two devices. These results show that SC-islets generated by methods generated using compositions and methods of the disclosure, including regimen 2 and regimen 1, can exhibit efficacy in increasing insulin production upon transplantation into diabetic subjects.

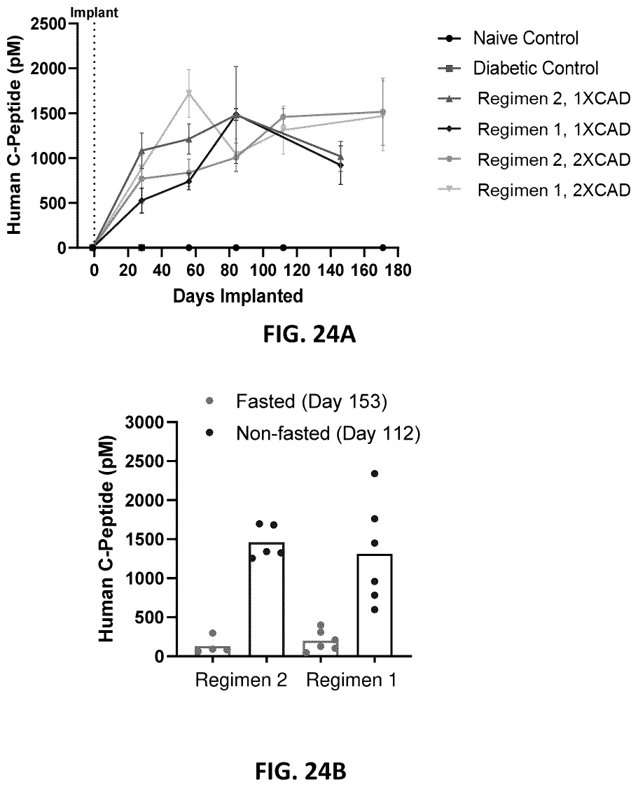

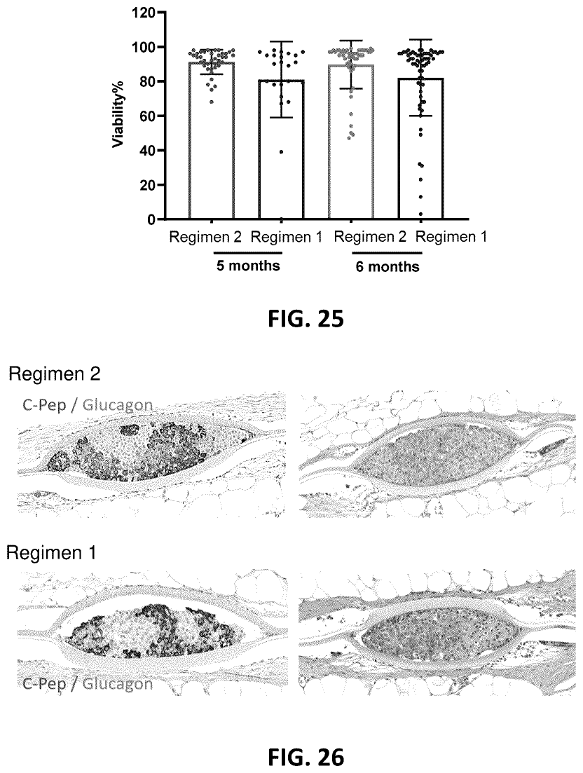

SC-islets were evaluated for cell viability and for C-peptide and glucagon production at 5 months (1 device) or 6 months (2 devices) post-implant. FIG. 25 provides viability data, showing high cell viability for devices with cells generated by either the regimen 2 or regimen 1 at stage 6 of differentiation. FIG. 26 provides illustrative histology images of sections stained for C-peptide and glucagon (left panels) or H&E stained (right panels). These results show that SC-islets generated using compositions and methods of the disclosure, including regimen 2 and regimen 1, can remain viable after implantation, and are capable of producing insulin and glucagon.

1. An in vitro composition comprising a plurality of PDX1-positive, NKX6.1-positive, insulin-negative cells, and one or more of an acetyl CoA related metabolite, an HDAC inhibitor, a redox homeostasis regulator, or a one carbon metabolism pathway intermediate.

2. An in vitro composition comprising a plurality of PDX1-positive, NKX6.1-positive, insulin-negative cells and an acetyl CoA related metabolite.

3. An in vitro composition comprising a plurality of PDX1-positive, NKX6.1-positive, insulin-negative cells, and one or more of acetate, β-hydroxybutyrate, taurine, or formate.

4. An in vitro composition comprising a plurality of PDX1-positive, NKX6.1-positive, insulin-negative cells and acetate.

5. The composition of claim 3, wherein the composition comprises between 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate.

6.-7. (canceled)

8. An in vitro composition comprising a plurality of PDX1-positive, NKX6.1-positive, insulin-negative cells and 3-10, 3-7, 3-8, 3-6, 3-5, 3-4, 3.5-4.5, 3.8-4.2, or 3.9-4.1 mM glutamine.

9. The composition of claim 8, wherein the glutamine is at a concentration of 3.8-4.2 mM.

10. (canceled)

11. The composition of claim 8, wherein at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in an alanine-glutamine dipeptide form.

12.-13. (canceled)

14. An in vitro composition comprising a plurality of insulin-positive endocrine progenitor cells and 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of an acetyl CoA related metabolite.

15. An in vitro composition comprising a plurality of insulin-positive endocrine progenitor cells and 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM of acetate.

16. (canceled)

17. An in vitro composition comprising a plurality of insulin-positive endocrine progenitor cells and 3-10, 3-7, 3-8, 3-6, 3-5, 3-4, 3.5-4.5, 3.8-4.2, or 3.9-4.1 mM glutamine.

18.-19. (canceled)

20. An in vitro composition comprising a plurality of insulin-positive endocrine progenitor cells and one or more of an acetyl CoA related metabolite, an HDAC inhibitor, a redox homeostasis regulator, a one carbon metabolism pathway intermediate, glutamate, or L-carnitine.

21.-22. (canceled)

23. The composition of claim 20, wherein the composition comprises β-hydroxybutyrate.

24. The composition of claim 20, wherein the composition comprises taurine.

25. The composition of claim 20, wherein the composition comprises formate.

26. The composition of claim 20, wherein the composition comprises a vitamin.

27. The composition of claim 20, wherein the composition comprises biotin.

28. The composition of claim 20, wherein the composition comprises glutamine, acetate, β-hydroxybutyrate, taurine, formate, and biotin.

29. The composition of claim 28, wherein the composition further comprises L-carnitine.

30. The composition of claim 20, wherein the composition comprises L-carnitine.

31.-32. (canceled)

33. The composition of claim 20, wherein the composition further comprises a ROCK inhibitor.

34. The composition of claim 33, wherein the ROCK inhibitor is thiazovivin, Y-27632, Fasudil/HA1077, or 14-1152, or derivatives thereof.

35.-68. (canceled)

69. The composition of claim 20, wherein the composition comprises at least 35%, at least 38%, at least 40%, at least 42%, at least 44%, or at least 46% ISL1-positive, NKX6.1-positive cells.

70.-74. (canceled)

75. A method comprising contacting a plurality of PDX1-positive, NKX6.1-positive, insulin-negative cells in vitro with a first composition that comprises one or more of an acetyl CoA related metabolite, an HDAC inhibitor, a redox homeostasis regulator or a one carbon metabolism pathway intermediate.

76. A method comprising contacting a plurality of PDX1-positive, NKX6.1-positive, insulin-negative cells in vitro with a first composition that comprises an acetyl CoA related metabolite.

77. A method comprising contacting a plurality of PDX1-positive, NKX6.1-positive, insulin-negative cells in vitro with a first composition that comprises one or more of acetate, β-hydroxybutyrate, taurine, or formate.

78. A method comprising contacting a plurality of PDX1-positive, NKX6.1-positive, insulin-negative cells in vitro with a first composition that comprises acetate.

79.-81. (canceled)

82. A method comprising contacting a plurality of PDX1-positive, NKX6.1-positive, insulin-negative cells with a first composition that comprises 3-10, 3-7, 3-8, 3-6, 3-5, 3-4, 3.5-4.5, 3.8-4.2, or 3.9-4.1 mM glutamine.

83.-131. (canceled)

132. A method comprising contacting a plurality of insulin-positive endocrine progenitor cells in vitro with a first composition that comprises one or more of an acetyl CoA related metabolite, an HDAC inhibitor, a redox homeostasis regulator, a vitamin, a one carbon metabolism pathway intermediate, L-carnitine, or glutamate.

133. A method comprising contacting a plurality of insulin-positive endocrine progenitor cells in vitro with a first composition that comprises one or more of glutamate, acetate, β-hydroxybutyrate, taurine, biotin, L-carnitine, or formate.

134.-182. (canceled)

183. A method comprising:
(a) contacting a plurality of PDX1-positive, NKX6.1-positive, insulin-negative cells with a first composition in vitro, wherein the first composition comprises acetate, glutamine, and one or more of β-hydroxybutyrate, taurine, formate, or biotin, thereby generating a first population of cells that comprises a plurality of cell clusters that comprise insulin-positive endocrine progenitor cells;
(b) dissociating at least a portion of the plurality of cell clusters in the first population of cells in vitro; and
(c) contacting the first population of cells comprising at least a portion of the dissociated cell clusters with a second composition in vitro, wherein the second composition comprises one or more of glutamate, acetate, β-hydroxybutyrate, taurine, biotin, L-carnitine, or formate, thereby generating a second population of cells comprising a plurality of cells clusters comprising a plurality of insulin-positive cells.

184. The method of claim 183, further comprising:
contacting the second population of insulin-positive cells in vitro with a third composition, wherein the third composition is different from the second composition, thereby differentiating at least a portion of said second population of insulin-positive cells into a third population of cells comprising a plurality of β cells.

185.-194. (canceled)

195. A method comprising contacting a population of NKX6.1-positive, ISL1-positive, insulin-positive cells with one or more of a serum albumin protein, a TGF-β signaling pathway inhibitor, a TH signaling pathway activator, a protein kinase inhibitor, a ROCK inhibitor, a BMP signaling pathway inhibitor, an epigenetic modifying compound, acetyl CoA-related metabolite, a vitamin, histone deacetylase inhibitor (HDACi), a redox homeostasis regulator, a one carbon metabolism pathway intermediate, glutamate, and/or carnitine for a first period of 1, 2, 3, 4, 5, 6, or 7 days.

196. (canceled)

197. A method comprising contacting a population of NKX6.1-positive, ISL1-positive, insulin-positive cells with one or more of HSA, Alk5 inhibitor II, GC-1, staurosporine, thiazovivin, LDN193189, DZNEP, taurine, acetate, betahydroxybutyrate, biotin, carnitine, glutamate, and formate for a first period of 1, 2, 3, 4, 5, 6, or 7 days.

198.-208. (canceled)

209. A composition comprising at least a portion of the third population of cells of claim 184.

210. A device comprising the cells of claim 209.

211.-213. (canceled)

214. A method of treating a subject with a disease characterized by high blood sugar levels over a prolonged period of time, the method comprising administering the cells of claim 209 to the subject.

215. (canceled)

216. A composition comprising a plurality of insulin-positive cells and a cryopreservative, and one or more of an acetyl CoA related metabolite, an HDAC inhibitor, a redox homeostasis regulator, a one carbon metabolism pathway intermediate, a vitamin, or L-glutamine.

217.-230. (canceled)
